SEQUENCE LISTING (95 sequences)

SEQ ID NO: 1 - DNA, 862 bp, Human immunodeficiency virus type 2
tgctagggattttcctgcttaggtttcccaaagcaagaagggtcctaacagaccagggtcctttattcagctgaacaccgagtgaccaggcggcgactaggagagatgggaacacacacttgacttgcttctaattggcagctttattaagagggttttaagcaagcaagcgtggagccgtctgcccagcaccggccaagtgctggtgagagtccagcagggaacacccaggctctacctgctagtgctggagagaacctcccagggctcaatctgccagcctctccgcagagcgactgaatacaatgcaagaagcgggtacatttatacagaggcttaatgggcgttccccacctactcctcccatgtccctcccactgttacagccccttctggaaagtccctgcagtgtcagccagtttcctttcttatgcagcgtcagctagttcctctttatgctgctgctttcctgtctaccttgtaggtatgccccttgcttttagtctagccttccattcctcctctggtaatcctgactgatacccaaactcttctgggaacctgttgaaggcctcatatgaatatgccaggagggaatcaaactgccagacaagagtctccccatggggatcatcccatgcggcggtctgtgctgggtgcactagacaatgggtctcctcctcctcccgggttgctgttgccacttccaccggcactagtttccacagccagccaaagtattttgggtatcttatccctggcccatatgtgtagtcctgccatccaggaactataccttcctcattctcaaaatatgtatctaatatcctatgtcttctttcactataaaaaatcccttcca

SEQ ID NO: 2 - DNA, 849 bp, Human immunodeficiency virus type 2
ggattttcctgccttggtttcccaaagcaagaagggtcctaacagaccagggtcttgttactcagatgaacaccgaatgaccaggcggcgactaggagagatgggaacacacacttaacttgcttctaatttggcagctttattaagaggactttaagcaagcaagcgtggagccgtctgcccagcgccggccaagcgctggtgagagtctagcagggaacacccaggctctacctgctagtgctggagagaacctcccagggctcaatctgccagcctctccgcagagcgactgaagtacaatgcaagaagcgggtacatttatacagaaagtatgagggccttccccaccagctcctcccatgtccctcccctggttacagccccttctggaaagtccctgcagtctcagctgttttcagtagctacttcctgccctgaccaagtatagctgttcctgtctctcagttaaatggaattccccttgctttcagtctcgccttccactcctcttctggtaggcctgactggtacccaaattcctctgggtgtaggataaaggcctggtacttgagggccagcatggggtcaaacttccacattagtgtttccccatggtggtcatcatacctacttgtctgtgctgggtgtgtcagacagttagcctcattgtcctcatcctcttgtgagatatctactggtactagcttccacagccacccaaagaacttagggtatcttactcctggcccatgagtataattctgccaatctggaattatcccttcttccttttctaaatatatatctaggattttatgtcttctttcactataaaaaatcccttcc

SEQ ID NO: 3 - DNA, 846 bp, Human immunodeficiency virus type 2
ggaagggatgttttacagtgagagaagacatagaatcttagatatatacttagaaaaggaagaagggataattccagattggcagaactatactcatgggccaggagtaagatacccaaagttctttggatggctatggaagctagtaccagtaaatgtcccacaagaaggggaggacactgagacccactgcttaatgcacccagcacaaataagcaggtttgatgacccgcatggggagacactagtctggcagtttgatcctatgctggcttataattatgaggcttttattcgacacccagaggaatttgggtacaagtcaggcctaccagaagaagagtggagggcgagactgaaagcaagaggaataccatttagttaaagacaggaactgctatacttggtcagggcaggaagtagctgctgaaaacagctgagactgcagggactttccagaaggggctgtaccaggggagggacatgggaggagctggtggggaacgccctcatactttctgtataaatgtacccgctgctcgcactgtatttcagtcgctctgcggagaggctggcagattgagccctgggaggttctctccagcactagcaggtagagcctgggtgttccctgctagactctcaccagtgcttggccggcactgggcagacggctccacgcttgcttgcttaaagacctcttaataaagctgccaattagaagcaagttgagtgtgtgctcccatctctcctagtcgccgcctggtcattcggtgttcacctggataacaagaccctggtctgttaggacccttcttgctttaagaaaccaaggcaggaaaatcc

SEQ ID NO: 4 - DNA, 839 bp, Human immunodeficiency virus type 2
ggattttcctgccttggtttcccaaagcgggaagggtcctaacagaccagggtcttgttactcaggtgaacaccgaatgaccaggcggcgactaggagagatgggaacacacacttgacttgcttctaactggcagctttattaagaggtttttaagcaagcaagcgtggagtcgtctgcccagtgccggccaagcactggtgagagtctagcggggagcacccgggctctacctgctagtgctggagagaacctcccagggctcaatctgccagcctctccgcagagcgactgattacaatgcaagaagcgagtacatttatacagtaggtatgagggcgttccccaccagctcctcccatgtccctcccatggttacagccccttctggaaagtccctgcagtctcagctgtttctacttcctgctctgaccaagtatggttgttcctgttattcactatatggtatccctcttgctttcagttttgccttccactctttctctggcaattctgacttatgcccaaattcttctgggtgtagaatgaaggccttgtaactattggccagcatggagtcaaactgccaaaccagtgtctccccatgtatgtcatcatatctgcttgtttgtgctgagtgcattaagcagttggtctcatcttcctcctcctcttgtgacatgtttactggtactagcttccacaaccacccgaagaacattgggtaccttactcctggcccatgagtataattctgccagtctggtattattccttcctccctttctaggtacatgtctaggattctatgcctatcctcgctgtaaaacatcccttcc

SEQ ID NO: 5 - DNA, 847 bp, Human immunodeficiency virus type 2
ggaagggattttttatagtgaaagaagacataaaatcctagatatatacctagaaaaggaagagggaataattccagactggcagaactacacttatgggccaggagtaagatatccaatgttctttgggtggctgtggaagctagtaccagtagatgtcccacaggaggaagaggagaatgactgtaactgcttactacacccagcacaaaccagcaagtatgatgacccacatgggcagacactggtttggagatttgaccctatgctggctcatgactacatggcctttaatctacacccagaggaatttgggcataagtcaggactgccagaagaagaatggaaggcaaaactgaaagcaagagggataccatttagttgaaaacaggaacagctatacttggtcagggcaggaagtaactactaaaaacagctgagactgcagggactttccagaaggggctgtaaccaagggagggacatgggaggagctggtggggaacgccctcatactttctgtataaatgtacccgctgctcgcattgtattcagtcgctctgcggagaggctggcagattgagccctgggaggttctctccagcactagcaggtagagcctgggtgttccctgctggactctcaccagtgcttggccggcactgggcagacggctccacgcttgcttgcttaaaagacctcttaataaagctgccagttagaagcaagttaagtgtgtgttcccatctctcctagtcgccgcctggtcattcggtgttcaccagaataacaagaccctggtctgttaggaccctttctgctttgggaaacggaggcaggaaaatcc

SEQ ID NO: 6 - DNA, 847 bp, Human immunodeficiency virus type 2
gcaagggatgttttacagtgaaagaagacataaaattgtagatatatacttagaaaaggaagaggggatagctgcagattggcagaattatactcatgggccaggaacaagatacccaatgttctttgggtggctatggaagctagtaccagtagatatcccacaagaaggggaggacactgagactcactgcttaatgcacccagcacaaacaagcaagtttgatgacccacataaggaggcactagtttggaggtttgaccccatgctggcctttagatacgaggccttcatccgacacccagaggaatttgggcacaagtcagggctaccagaagaagagtggaaagcgagactgaaagcaaggggaataccatttagttaaagacaggaactgccatacttggtctgggcaggaagtagctactgaaaacagctgagactgcagggactttccagaaggggctgtaaccaggggagggacatgggaggaactggtggggaacgccctcatactttctgtataaatgtacccgctacttgcattgtacttcagtcgctctgcggagaggctggcagattgagccctgggaggttctctccagcactagcaggtagagcctgggtgttccctgctagactctcaccagtgcttggccggcactgggcagacggctccacgcttgcttgcttaaaaacctcttaataaagctgccagttagaagcaagttaagtgtgtgctcccatctctcctagtcgccgcctggtcattcggtgttcacctgaataacaagaccctggtctgttaggacccttcttgctttgggaaaccaatgcaggaaaatcc

SEQ ID NO: 7 - DNA, 846 bp, Human immunodeficiency virus type 2
ggaagggatgttttacagtgagagaagacatagaatcctggacatatacttagaaaaggaagaggggataattgcagactggcagaactatactcatgggccaggggtaagatacccgaaattctttgggtggctatggaagctagtaccagtagacaccccacaagagggggaggatgatgggactcactgcttgctacacccagcacaaacaggcaagtttgatgacccgcatggggagacactggtttggaggtttgaccataggctggcttacaagtacaaggcctttattctatacccagaagagtttgggtacaagtcaggcctgccagaggaggaatgaaaagcaagactgaaagcaagagggataccattcagttaaaaacaggaacagctatacttggtcagggcaggaagtaactactgaaaacagctgagactgcagggactttccagaaggggctgtaaccaggggagggacatgggaggtactggtggggaacgccctcatactttctgtataaatgtacccgctgctcgcattgtattcagtcactctgcggagaggctggcagattgagccctgggaggttctctccagcactagcaggtagagcctgggtgttccctgctagactctcaccagtgcttggccggcactgggcagacggctccacgcttgcttgcttaaagacctcttaataaagctgccaattagaagcaagtcaagtgtgtgttcccatctctcctagtcgccgcctggtcattcggtgttcacctgagtaacaagaccctggtctgttaggaccctttctgctttggaaaaccaaggcaggaaaatcc

SEQ ID NO: 8 - DNA, 848 bp, Human immunodeficiency virus type 2
gcaagggatgttttacagtgaaagaagacatagaatcctagatatatacttagaaaaggaggaagggatacttgcagattggcaaaactatacttatgggccaggagtaagataccctaagttctttggatggctatggaagctagtaccagcagatgtcccacaagagggggaggacatcgagactcactgcttaatgcacccagcacaaataagcaactttgatgaccctcatggggagacattagtttggaggtttgaccccatgctggcctatgactacacagcctttaaacaatacccagaggaatttgggcacaagtcaggcctaccagaagatgaatggaaggcaagactgaaagcaaggggaataccatttgactgagagacaggaacagctatacttggtcagggcaggaagtagctactgaaaacagctgagactgcagggactttccagaaggggctgtaaccaggggagagacatgggaggagttggtggggaacgccctcatactttctgtataaatgtacccgctgcttgcattgtacttcagtcgctctgcggagaggctggcagattgagccccgggaggttctctccagcactagcaggtagagcctgggtgttccctgctagactctcaccagtgcttggccggcactgggcagacggctccacgcttgcttgcttaaagacctcttaataaagctgccaattagaagcaagttaagtgtgtgctcccatctctcctagtcgccgcctggtcattcggtgttcgcctgagtaacaagaccctggtctgttaggacccttcttgctttgggaaaccaatgcaggaaaatcc

SEQ ID NO: 9 - DNA, 849 bp, Human immunodeficiency virus type 2
ggaagggatgttttacagtgaaaggagacacagaatcttagacatgtacttagaaaatgaagaagggataataccagattggcagaactatagttatggaccaggaataaggtatccaatgttctttgggtggctatggaagctagtaccagtagaagtcccagaagagggggagaaagacagcgggactcactgcttactgcacccatcgcagacaagcaggtttgatgacccgcatggagaaacactagtctggaagtttgaccctatgctggctcatgaatacactgcttatattcgattcccagaggaatttaggcacaagtcaggtctgccagaagaagagtggaaggcaagactgaaagcaagagggataccatttagtgaataacaggaacaaccatacttggtcagggcaggaagtagctgctggaaacagctgagactgcagggactttccagaaggggctgtaaccaagggagggacatgggaggagctggtggggaacgccctcatactttctgtataaatgtacccgctactcgcattgtattcagtcgctctgcggagaggctggcagattgagccctgggaggttctctccagcactaacaggtagagcctgggtgctccctgctagactctcaccagtacttggccggtactgggcagacggctccacgcttgcttgcttaaagacctcttaataaagctgccagttagaagcaagttaagtgtgtgttcccatctctcctagtcgccgcctggtcattcggtgttcacctgaataacaagaccctggtctgttaggacccttcccgctttgggaaaccgaagcaggaaaatcc

SEQ ID NO: 10 - DNA, 984 bp, Human immunodeficiency virus type 2
cccttctcctcacgctgcttggtaccagcccgcgcctttacaggagctctccgtcgtggttgattcctgccgcccttactgccttcactcagctgtgctcctgaagactctcactcttctcaggtccctgttcgggcgccaatctgctagggattttcccactttggtttcccaaagcaagaagggtcctaacagaccagggtcctatatcagtggagcaccgagtgaccaggcggcgactaggagagatgggaacacacacttcacttgcttctaattggcagtttattaagaggtttttaagcaagcaagcggggagccgtctgcccagcaccggccaagtgctggtgagagtctagcagggaacacccaggctctacctgctagtgctggagagaacctcccagggctcaatctgccagcctctccgcagagcgactcaatacaatgcgagaagcgggtatatttatacagagatttaatgggcgttcccaaccaactcctcccatgcccctcccctgttaccgcctcctttggaaagtccctgcagtgtcagctagtttccctcctgtgcagtgtcagctagttcctgtttatgctgctgtttgcctagtctgtaggtatccctcttgcttttagtttagccttccactcctcctctggcagtcctgactggtacccaaactcctctggaaacttattgaaggccacataatcatatgccaggtaggaatcaaactgccagacaagagtctccccatgaggaggtctgtgccgggtgcactaggcaatgggtttcctcctcctcctgggcctctgctggcgtctttactggtactagcttccacagccaaccaaaggtcttgggccatcttacccctggcccatacgtatagttttgccagccaggcacaacgccttcttcattctcaaaatatgtatctaatattctgtgtcttctctcactatagtaaatcccttcca

SEQ ID NO: 11 - DNA, 792 bp, Human immunodeficiency virus type 2
tcagtggagcaccgagtgaccaggcgacgactaggagagatgggaacacacacttgtcttgcttctaataggcagctttattaagagggttttaagcaagcaagcgtggagccgtctgcccagcaccggccaagtgctggtgagagtctagcagggaacacccaggctctacctgctagtgctggagagaacctcccagggctcgatctgccagcctctccgcagagcgactgactacagtgcagaaagcgggtacatttatacagagtcttaatgggcgttcccctccaactcctcccatgtccctcccactgttactgccccttttggaaagtccctgcagtatcagctagtttcctttcttatgcagtgtcagctagttcctctttatgctgttatttcctgcctaatctgttggtattcctcttgctttcagtctagccttccattctttctctggtagtcctgactgatgcccaaactcctctgggtacctgttgaaggcctcatacttatatgctaggagggagtcaaactgccagacaagggtctctcggtgggggtcatcccatggggaggtctgtgctggatgcaccagacaatgggtttcctcatcctggggctctgctatcatatttactggtactagcttccataaccaaccaaaaaacatgggatatcttatccctggcccatgtgtatagttctgccatccagccacaatgcctctttcattttccatgtatgtgtctaatatcctatgtcttctttcactataaaaaatcccttcca

SEQ ID NO: 12 - DNA, 3186 bp, Human immunodeficiency virus type 2
cagaagacagggatgctggaaatgtggaaaaccaggacacaacatggcaaattgtccagaaagacaggctggttttttaggacttggaccctggggaaagaagcctcgcaacttccccatggcccaagtgcctcaggagatagtgccatctgcgcccccgatgaacacagcagagggcaagacacatcagggagcgataccatctgcgccccctgcagatccggcagtggagatgctgaaaagctacatgcaactagggaagcagcagagagggaagcaggggaggccctacaaggaggtgacagaggacttgctgcacctcaattctctctttggagaagaccggtagtccaagcatatatcgagggtcagccagtagaagtgttactagacacaggggctgacgactcaatagtagcaggaatagaattaggaagcaattacaccccaaaaatagtaggagggataggagggttcataaatactaaggaatacaaaaacatagaaatagaagtagtaggaaaaaaggtaagagcaactataatgacaggagacaccccaataaatattttcggcagaaatatcttaaacaccttaggcatgactctaaatttcccagtggcaaaaatagagccagtgaaggtccagttaaagcctgggaaggatggaccaaaaatcagacaatggcctctatctaaggaaaagatactggcccttagagaaatctgtgaaaaaatggaaaaggaggggcagctagaagaagcatctcccactaatccatacaacacacccacctttgctataaagaaaaaggataaaaacaaatggagaatgctaatagacttcagggaattaaataaggtaacccaagaatttacagaagtccaactaggtattccccacccggcagggctggcagaaaaaaggagaataacagtgatagatgtgggagatgcctacttcagtgtcccactagacccagacttcaggcaatatacagcattcactttgccatcaataaataatgcagagccaggaaaaagatacatttataaagtcctaccacagggatggaagggatccccagcaatttttcaatactccatgagaaaggtactagaccccttcagaagggccaacaatgatgtcaccataatccagtacatggatgacatccttgtggcaagcgatagaagtgacttagagcatgataaggtagtgtctcaactaaaagagctgctaaatgacatgggattttccaccccagaagaaaaatttcaaaaggaccctccattccagtggatgggttatgagctctggccaaagaagtggaagctgcaaaaaatacaaataccagaaagggaggtttggacagtaaatgacattcagaaactagtgggagtattaaactgggcagctcaacttttccccggaatcaaaacaaggcacatatgcagattgatcaagggaaagatgaccctgacagaggaggtgcagtggacagaactagcagaggcagagatgcaggaaaataaaatcattctaggacaggaacaggaaggatcctactacaaagaaggggtacctctagaagcaacagtgcagaaaaacttagcaaaccagtggacatacaaaattcatcagggagataaagtcctaaaagtaggaaaatatgcaaaggttaaaaacacccacaccaatggagtaagaatactagcgcatgtagtccaaaaaataggaaaagaagcattggttatttggggacaaataccaatgttccacctgccagtagaaagagagacatgggaccaatggtggacagattactggcaagtgacttggatcccagaatgggactttgtctcaaccccaccattggtaaggctagcctacaacctaatcaaggaccccctagaaaaggtagaaacctattacacagatgggtcctgcaacagaacctcaaaggaaggaaaggcaggatatgtcacagacaggggaagggacaaagttaaaccattagagcagacaacaaatcaacaagcagaacttgaagcatttgcgctagcactacaggattcaggaccacaagtcaacatcatagtagactcacaatatgtcatggggatagtagctggacagccaacagaaacagagtcaccaatagtaaataagataattgaagaaatgatcaaaaaggaagcaatatatgtaggatgggtgccagctcacaaaggactaggtggtaatcaggaagtagaccacctagtaagtcaaggaattaggcaggttctattcctagaaaagatagaaccagcacaagaggaacatgaaaaatatcatggcaatgtaaaagaactggttcataaatttggaattccacaactagtggcaaaacaaatagtaaactcctgtgataaatgtcaacaaaaaggagaagctgttcatggacaggtaaatgcagaactagggacatggcaaatggactgtacacatttagaagggaaagtcataatagtagcagtccatgtagccagtgggtttatagaagcagaggtaataccccaagaaacaggaagacaaacagccctcttcctgttaaagctggcaagcagatggcccattacacacttgcacacagacaatggtgcaaactttacttcacaagatgtaaagatggtggcttggtgggtaggaatagaacaaacctttggagtaccctacaacccacaaagtcagggagtagtagaagcaatgaaccatcacctaaaaaatcaaatagataggatcagagatcaggcagtatcaatagaaacagttgtgctaatggcagctcattgcatgaattttaaaagaaggggaggaataggggatatgacccctgcagagagattagttaacatgataaccacagaacaagaaatacaattcttccaagcaaaaaatttaaaatttcaaaatttccaggtctattacagagaaggcagagatcaactctggaagggacctggtgaactattgtggaaaggggaaggagcagtcatcataaaggtagggacagagatcaaagtaatacccagaagaaaagcaaagatcataaggcactatggagggggaaaagaattggattgtagtaccgacgtggaggataccaggcaggctagagaaatggcacagtctggtcaagta

SEQ ID NO: 13 - DNA, 2961 bp, Human immunodeficiency virus type 2
aacagcacccccagtagatccagcagtggacctattggagaaatatatgcagcaagggagagagcaaaaggagcagagaaggagaccatacaaggaagtgacagaggacttactgcacctcgggcaaggagagacaacatgcagggaaccgacagaggacttgctgcacctcaattctctctttggaaaagaccagtagtcacagcatacattgagactcagccagtagaagttttactagacacaggggctgacgactcaatagtagcagggatagagttaggaagcaattatagtccaaaaatagtagggggaatagggggattcataaataccaaggaatacaaaaatgtagaaatagaagttctaggtaaaaaagtaagggccaccataatgacaggtgataccccaatcaacatttttggcagaaatattttgacagccttaggcatgtcattaaacctaccagttgccaaaatagagccaataaagataatgctaaaaccaggaaaggatggaccaaaactgagacagtggcccttaacaaaagaaaaaatagaagcactaaaagaaatctgtgaaaagatggaaaaagaaggccagctagaagaagcacctccaactaacccttataatacccccacattcgcaatcaggaagaaggacaaaaataaatggaggatgctaatagatttcagagaactaaacaaggtaactcaagatttcacagaaattcagctaggaattccacacccagcaggactagccaagaagagaaggattactgtactagatgtaggggatgcctacttttccataccactacatgaagactttagacagtacactgcatttaccctaccatcagtgaacaatgcagaaccaggaaaaagatacatatataaagtcttgccacagggatggaagggatcaccagcaatttttcaatacacaatgaggcagatcttagaaccattcagaaaagcaaaccaggatgtcattatcattcagtacatggatgatatcttaatagctagtgacaggacagatctagaacatgacagagcagtcctgcagctaaaggaacttctaaatggcctagggttttctaccccagatgataagttccagaaagaccctccataccactggatgggctataaattatggccaactaaatggaagttgcagaaaatacaattgccccaaaaggaaacatggacagtcaatgacatccaaaagctagtgggtgtcctaaactgggcagcacaaatctacccagggataaagacaaaacacttatgcaaattaattaggggaaaattggcactcacagaagaagtacagtggacagagttagcagaagcagaactagaagaaaacagaatcatcttgagccaggaacaagagggacactattaccaagaagacaaagagttagaagcaacagtccaaaaggatcaggacaaccagtggacatataaagtacaccagggagaaaagattctaaaagtaggaaaatatgcaaaggtaaaaaatacccataccaatggagtcagactgttagcacaggtagttcagaaaataggaaaagaagcactagtcatttggggacgaataccaaaatttcacttaccagtagaaagggaagtctgggagcagtggtgggacaactactggcaggtgacgtggatcccagactgggacttcgtgtctaccccaccgctggtcaggttagcatttaatctggtaaaagaccctataccaggtgcagagacctactacacagatggatcctgcaataggcaatcaaaggaaggaaaagcaggatacataacagacagagggagagacaaggtaaaagtactagagcagactaccaatcagcaagcagaattagaagcctttgcaatggcattaacagattcaggtccaaaagctaatattatagtagactcacagtatgtaatgggaatagtggcaggccaaccaacagagtcagaaaataggatagtgaatcaaatcatagaagaaatgataaaaaaggaagcaatctatgtggcatgggtcccagcccacaaaggaataggaggaaatcaggaggtagatcatttagtgagtcagggcatcagacaagtattgttcctagagagaatagagccagctcaagaagaacatgaaaaattccatagcaatgtaaaagaactatcccataaatttggactacccaacttagtggcaagacaaatagtaaacacatgcccccaatgccaacagaaaggggaggctacacatgggcaagtaaatgcagaattaggcacttggcaaatggactgcacacatttagaaggaaaagtcattatagtagcagtacatgttgcaagtggatttatagaggcagaagtcatcccaaatgaaacaggaaggcaaacagcactcttcctgttaaaactggccagtaggtggccaataacacacttgcacacagataatggtgccaactttacttcacaggaagtaaagatggtggcatggtggacaggtatagaacaaacctttggagtaccttacaacccacaaagccaaggagtagtagaggccatgaatcaccacctgaaaaatcaaataagcagaatcagagatcaggcaaatacagtagaaacaatagtactaatggcagttcattgcatgaattttaaaagaaggggaggaataggggatatgaccccatcagaaagactaatcaatatgatcacagcagaacaagaaatacagttcctccaagccaaaaattcaaaattaaaaaattttcgggtctatttcagagaaggcagagatcagttgtggaaaggacctggggagctactgtggaagggagacggagcagtcatagtcaaggtaggaacagacataaaaataataccaagaagaaaagccaagatcatcagagactatggagga

SEQ ID NO: 14 - DNA, 2961 bp, Human immunodeficiency virus type 2
cacagcacccccagtagatccagcagtagacctactggagaaatatatgcagcaagggaagaagcagagagagcagagacagagaccttacaaggaagtaacagaggacctactgcacctcgagcagggggagacaccatacagggagacaacagaggacttgctgcacctcaattctctctttggaaacgaccagtagtcacagcacacattgagggtcagccagtagaagtcttgctggacacgggggctgacgactcaatagtagcagggatagagttagggagcaattatagtccaaaaatagtagggggaatagggggattcataaataccaaggaatataacaatgtagaaatagaagttctaggtaaaagggtaagggccaccataatgacaggcgacaccccaatcaacatttttggcagaaatattctaacagccttaggcatgtcattaaatctaccagtcgccaaggtagaaccaataaaaataatgctaaagccaggaaaggatggaccaaaattgagacaatggccattaacaaaagaaaaaatagaagcactgaaagaaatctgtgaaaaaatggaaaaggaaggccagctaggggaagcacctccaactaatccttataatacccccacatttgcaatcaggaaaaaggacaaaaacaaatggaggatgctaatagatttcagagaactaaataaggtaactcaggatttcacagaaatccaattaggaattccacacccagcaggactagccaagaagagaagaattactgtactagatgtaggggatgcttacttttccataccattgcatgaggattttagaccatataccgcatttactctaccatcagtaaataattcagaaccaggaaagagatacatatataaggtcttgccacagggatggaaggggtcaccagcaatttttcaatacacaatgagacaggtcttagaaccattcagaaaagcaaacacagatgtcattatcattcagtatatggatgatattttaatagctagtgacaggacagatttagaacatgacagggtagtcctgcagctaaaggaacttctaaatggcctaggattttctaccccagatgacaagttccaaaaagaccctccataccactggatgggctatgaactatggccaactaaatggaagctgcagaaaatacagttgccccaaaaggacgcatggacagtcaatgacatccagaagctagtgggtgtcctaaattgggcagcacaactctacccagggataaagaccaaacacttatgcaggttaattagaggaaaactgacactcacagaagaggtacagtggacagacttagcagaagcggagctagaagaaaacaagattatcttaagccaggaacaagagggacattactaccaagaggaaaaagagttagaagcaacagtccaaaaggatcaaagcaatcagtggacatataaagtacaccagggagacaaaattctaaaagtaggaaagtatgcaaaggtaaaaaatacccataccaatggggtcagattgttagcacaggtagttcaaaagataggaaaagaagcactagtcatttggggacgaataccaaaatttcacctaccagtagaaagagaaacttgggagcagtggtgggataactattggcaagtgacatggatcccagactgggatttcgtatctaccccaccactggtcaggttagcatttaacctagtaggagatcctataccaggcacagagactttctacacagatggatcctgcaataggcaatcaaaagaaggaaaagcaggatatgtaacagatagagggagagacaaggtaaaagtactagagcaaactaccaatcagcaagcagaattagaagcctttgcaatggcactaacagactcgggtccaaaagttaatatcatagtagactcacagtatgtaatgggaatagtagcaggccaaccaacagaatcagagaatagaatagtaaatcaaatcatagaagaaatgatcaaaaaagaagcaatctatgttgcatgggtcccagcccacaagggcataggaggaaatcaggaagtagatcatttagtgagtcagggcatcagacaaatattgttcctggaaaaaatagagcccgctcaggaagaacatgaaaaatatcatagcaatgtaaaagaactgtcccacaaatttggaatacccaagctggtggcaaggcaaatagtaaacacatgtgcccaatgtcaacagaaaggggaggctatacatgggcaagtaaatgcagaattaggcacctggcaaatggactgcacacacttagaaggaaaaatcattatagtggcagtacatgttgcaagtggatttatagaagcagaggtcatcccacaagaatcaggaaggcaaacagcactcttcctactgaaactggcaagtaggtggccaataacacacttgcacacagataatggtgccaacttcacttcacaggaagtgaaaatggtagcatggtgggtaggcatagaacaatcctttggagtaccttacaatccacagagccaaggagtagtagaagcaatgaatcaccacctaaaaaatcagataagtagaattagggaccaggcaaatacagtggaaacaatagtgctaatggcagttcattgcatgaattttaaaagaaggggaggaataggggatatgaccccatcagaaagattaatcaatatgataaccacagaacaagagatacaattcctccaagccaaaaattcaaaatttaaaaatttttcgggtctatttcagagaaggcagagatcagttgtggaaaggacctggggagctactgtggaagggagaaggagcagtcatagccaaggtaggaacagacataaaaataataccaagaagaaaagccaagatcatcagagactatggagg

SEQ ID NO: 15 - DNA, 2952 bp, Human immunodeficiency virus type 2
aacagcacccccagtagatccagcagtggacctgttggaaaaatatatgcagcaagggaaaaagcagagggagcagagggagaaaccatacaaggaggtgatggaagacttactgcacctcgaagagacgccccacagggaggcgacagaggacttgctgcacctcaattctctctttggaaaagaccagtagttacagcatacatcgaggggcagccggtagaagtcctactggacacaggggctgatgactcaatagtagcagaaatagagttaggggataattacactccaaaagtagtagggggaatagggggattcataaacaccaaagaatataaaaatgtagaaataaaagtactaaataaaagagtaagagccaccataatgacaggagacaccccaatcaacatttttggcagaaatattctgacagccttaggcatgtcattaaatttaccagttgccaagatagagccaataaaagtaacattgaagccagggaaggaagggccaaggctgaaacaatggcccctaacaaaagaaaaaatagaagcactaaaagaaatctgtaagaaaatggaaaaagagggccaactagaagaggcacccccaactaatccttataatacccccacatttgcaattaggaaaaaggacaagaacaaatggagaatgctaatagattttagggaactaaacaaggtgactcaagatttcacagaaattcaactaggaattccacacccggcaggattagccaaaaagaaaagaatcactgtattagatgtaggggatgcctacttttccataccactacatgaagattttagacagtatactgcatttaccctaccatcagtaaacaatgcagaaccaggaaagagatatatatataaagtcttaccacaaggatggaagggatcaccagcaatttttcaatacacaatgaggcaagtcttagaacctttcagaaaagcaaacccagatgtcattctcatccaatacatggatgatatcttaatagctagtgacaggacaggtttggagcatgacaaagtggtcctgcagctaaaagaacttctaaatggcctggggttctctaccccagatgagaagtttcaaaaagaccctccgttccaatggatgggctatgaactgtggccaactaaatggaagctgcagaaattacagctgccccagaaagagatatggacagtcaatgacatccaaaaactagtgggagtcttaaattgggcggcacagatctatccaggaataaaaaccaaacacttatgcaggctaattagaggaaaaatgacactcacagaagaagtgcagtggacagagttagcagaggcagagctagaagaaaacaaaattatcttaagccaggaacaagaaggacactattaccaagaagaaaaagaactagaggcaacaatccaaaaaagtcaagacaatcaatggacatacaaaatacaccaggaagaaaaaatcctaaaagtaggaaaatatgcaaagataaaaaatacccataccaatggggttagattactagcacaggtagttcagaaaataggaaaagaagcactagtcatatggggacgaataccaaaatttcacctaccggtggaaagagaaacctgggaacagtggtgggataactactggcaggtgacatggatcccagagtgggacttcgtatctaccccgccactggtcaggttgacatttaacctagtaggagatcctataccaggcacagagaccttctacacagatggatcatgcaatagacagtcaaaggaaggaaaagcgggatatgtaacagatagagggagagacaaggtaaaagtattagaacaaactaccaatcagcaagcagaattagaagcctttgcaatggcactggcagactcaggtccaaaggttaatatcatagtagactcacagtatgtaatggggatagtagcaagccaaccaacagagtcagaaagtaaaatagtaaaccaaatcattgaggacatgacaaagaaagaagcagtctatgttgcatgggttccagcccataagggcataggaggaaaccaggaagtagaccatttagtaagtcagggcatcagacaagtacttttcctggaaaaaatagagcccgcccaagaggaacatgaaaaatatcatagcaatataaaagaactgacccataaatttggaataccccaactagtagcaagacagatagtaaacacatgtgcccaatgccaacagaaaggagaggccatacatgggcaagtaaatgcagaaataggtgtttggcaaatggactgcacacacttagaaggaaaaatcattatagtagcagtacatgttgctagtggattcatagaagcagaggtcatcccacaggaatcgggaagacagacagcactcttcctgttaaaactggccagtaggtggccaataacacacttgcacacagacaatggcgccaacttcacttcacaggaagtgaagatggtggcatggtggataggtatagagcaatcctttggggtaccttacaacccacaaagccagggagtagtagaagcaatgaatcaccacttaaaaaatcagataagtagaattagagaacaggcaaatacaatagaaacaatagtactaatggcagttcattgcatgaattttaaaagaaggggaggaataggggatatgaccccggcagaaagactaatcaacatgattaccacagaacaagaaatacaattcctccaaagaaaaaattcaaattttaaaaaatttcaggtctattacagagaaggcagagatcagctgtggaaaggacctggagaactactgtggaagggagacggagcagtcatagtcaaggtaggggcagacataaaagtagtaccaagtaggaaagccaagataagggcgaattcgttt

SEQ ID NO: 16 - DNA, 2955 bp, Human immunodeficiency virus type 2
aacagcacccccagtggatccagcagaggacctgctggagaaatatatgcagcaagggagaaggcagaaagagcagagagagagaccatacaaagcagtgatggaggacttactgcaactcgagcagggagagacaccacacgggggggcgacagaggacttgctgcacctcaattctctctttggaaaagaccagtagtcacagcacacattgagggtcagccagtagaagttttactagacacaggggctgacgactcaatagtggcaggaatagagttagggagtgattatagtccaaaaatagtagggggaataggaggattcataaataccaaggaatataaaaatgtaaaaataagagtactaaataaaaaggtaagagccaccataatgacaggtgataccccaattaacatttttggcagaaacatcctggcaactttgggcatgtcattaaatttaccaatcgccaaggtagagccaataaaagtaacactgaagccaggaaaagatggaccaaaattgagacaatggcccttaacaagagagaaaatagaagcactaaaagaaatctgtgaaaaaatggaaaaagagggccagctagaaatagcatccccaactaatccttttaattcccccacatttgcaattaggaaaaaggacaaaaacaaatggaggatgctaatagactttagagaactaaacaaagtgactcaagattttacagaaattcagttgggaattccacacccagcaggattagccaagaagagaagaattactgtactagatgtaggggatgcctacttttccatacctctacatgaggattttagacagtatactgcatttaccctaccatcagtgaacaatgcagaaccaggaaaaaggtatatatataaagtcttgccgcagggatggaagggatcaccagcaatctttcaattcatgatgaggcagatcttagagccattcagaaaagcaaaccaggatgtcattatcatccagtacatggatgatatcttaatagctagcgacaggacagatttagaacatgacagagtggtcctacagctaaaagaacttctaaatggcctgggattttccaccccagatgagaaattccaaaaggaccctccatatcaatggatgggctatgagctatggccaactaagtggaagctacagaaaatacagttgccccaaaaggaaatatggacagtcaatgacatccaaaaactagtaggtgtcctgaactgggcagcacaactctacccagggataaagaccaaacacttatgtaagttaattagaggaaaattgacactcacagaagaagtacagtggacagaactagcagaagcagagttacaagaaaacaaaattatcttaagccaggaacaagagggatgctattaccaagaagaaaaagagttagaagcaacaatccaaaaggatcaagacaatcagtggacatataaaatacatcagggagaaaagatcctaaaagtaggaaaatatgcaaaagtaaaaaatacccatactaacggggtcagattgttagcacaggtaattcaaaaaataggaaaggaagcactagtcatttggggacgaataccaaaatttcacctaccagtagaaagagaaacctgggaacagtggtgggatgactactggcaagtgacatggatcccagactgggactttgtatctaccccaccactggtcaggttagcatttaacctagtgaaagatcctataccagacgtagagaccttctacacagatggatcctgcaataggcaatcaagagaaggaaaagcaggatacataacagatagaggaagagacaaggtgagagtactagaacaaactaccaatcagcaatcagaattagaagcctttgcaatggcagtaacagactcaggtccaaaagtcaacattatagtagactcacagtatgtaatgggaatagtagcaggccagccaacagaatcagagagtaaaatagtaaatcaaatcatagaagagatgataaaaaaggaagcaatctatgttgcatgggttccagcccataaaggcataggaggaaatcaggaagtagatcatttagtaagtcaaggcatcagacaagtactattcctagaaaaaatagagcccgctcaggaagaacatgaaaaatatcatagcaatataaaagaactgtcccataaatttggactacccaaactagtggcaaaacaaatagtgaactcatgtgcccaatgccaacagaaaggggaggccacacatgggcaagtaaatgcagaactaggcacttggcaaatggactgcacacacttagaaggaaaaatcatcatagtggcagtacatgttgcaagtgggtttatagaagcagaagtcatcccacaggagtcaggaaggcaaacagcactcttcctgttaaagctggccagtaggtggccaataacacacttacacacagataatggtgccaacttcacctcacaggaagtgaagatggtagcatggtgggtaggcatagaacaatccttcggagtgccttacaatccacaaagccaaggagtagtagaagcaatgaatcaccatctaaaaaatcagatagacagaattagagagcaagcaaatacaatagaaacaatagtattaatggcagtccattgcatgaattttaaaagaaggggaggaataggggatatgaccccagcagaaagactaatcaatatgatcaccacagaacaagaaatacaattcctccaagcaaaaaattcaaaattaaaaaattttcgggtctatttcagagaaggcagagatcagctgtggaaaggacctggggaactactgtggaagggagacggagcagtcatagtcaaggtaggggcagacataaaaatagtaccaagaaggaacgcaaagatcatcattgactat

SEQ ID NO: 17 - DNA, 2940 bp, Human immunodeficiency virus type 2
aacagcacccccagtagatccaatagaggacctactggagaaatatatgcagcaaggaaaaaggcagagagaacagagagagagaccatacaaggaggtgacggaggacttactgaacctggggcagggggagacaccatgcaaggagacgacagaggacttgctgcacctcaattctctctttggaaaagaccagtagtcacagcacacgttgagggccagccagtggacgtcttgctagacacaggggctgacgactcaatagtggcaggaatagagttagggtgcaattatactccaaagatagtagggggaataggaggattcataaataccaaagaatataaaaatgtagaaatagaagttctaggtaagagggtaagggctaccataatgacaggcgacaccccaatcaacatttttggcagaaatattctgacagccttaggcatgtcattaaatctaccagtcgccaaaatagaaccaataaaaataatgctaaagccaggaaaagatgggccaaagctgaggcaatggcccttaacaaaagaaaagatagaggcactaaaagaaatctgtgaaaaaatggaaaaagaaggccagctagaggaagcatctccaaccaatccttacaatacccccacatttgcaatcagaaagaaggacaagaataaatggagaatgctaatagatttcagagaactaaacaaggtaactcaagatttcacagaaattcagttaggaattccacacccagcaggactggctaaaaagaggagaatcactgtactagatataggggatgcttacttttccataccattacatgaggactttagacaatatactgcatttactttgccagcagtgaacaatgcagaaccaggaaaaagatatatatataaagtcttacctcagggatggaaaggatcaccagcaatttttcaatacacaatgaggcagatcctagaaccattcagaagagcaaacccagatgtcattatcattcagtacatggatgatatcttaatagctagtgacaggacagacttagaacatgacaaggtggtcctgcagctaaaggaacttctaaatggcctaggattttctaccccagatgagaagttccagaaagaccctccataccgctggatgggctatgaattgcggccaactaaatggaagctgcagaaaatacagttgccccaaaaggaagtatggacagtcaatgacatccaaaaactagtgggtgtcctaaattgggcagcacaaatttacccaggaataaaaaccaaacacttatgtaggttaattagaggaaaaatgacactcacagaagagatacagtggacagacttagcagaagcagaactagaggagaacaaaattatcctaagccaggaacaagagggacactattaccaagaggacaaagagttagaagcaacagtccaaaaagatcaagacaatcagtggacatataaagtacaccagggagaaaaaatcttaaaggtaggaaggtatgcaaaggtaaaaaatacccataccaacggggtcagattgttagcacaggtagtacaaaaaataggaaaagaagcactagtcatttggggtcggataccaaaatttcacctaccagtagagagagaaacctgggagcagtggtgggatgactactggcaagtaacatggatcccagactgggatttcgtgtctaccccaccactggtcaggttagcattcaacctagtaaaagatcctataccaggtacagagactttctacacagatggatcctgcaataggcaatcaaaagaaggaaaagcaggatatataacagatagagggagagacaaggtaaaagtgctagaacaaactaccaaccagcaagcagaattacaagcctttgcaatggcactaacagactcaggcccaaaagctaatatcgtagtggactcacagtacgtaatgggaatagtagcaggccaaccaacagagtcagaaagtagaatagtaaatcaaatcatagaggaaatgataaaaaaggaagcaatctatgttgcatgggttccagcccataagggcataggaggaaatcaggaggtagatcatttagtgagtcagggcatcagacaagtactgttcctagaaaaaatagagcctgctcaggaagagcatgaaaaatatcacagcaatgtaaaagaactatcccataaatttggattacccaaactagtagcaagacaaatagtaaacacatgtactcaatgtcagcaaaagggagaagctatacatgggcaagtgaatgcagaattaggcacttggcaaatggactgcacacacttagaaggaaaagtcattatagtagcagtacatgttgcaagtgggtttatagaagcagaagtcatcccacaggaatcaggaaggcagacagcactcttcctattgaaactggccagtaggtggccaataacacacttacacacagataatggtaccaacttcacttcacaggaggtaaagatggtagcatggtgggtaggtatagaacaaacctttggagtaccttacaatccacaaagccaaggagtagtagaagcaatgaatcaccacctaaaaaatcagataaatagaattagagaacaggcaaacacagtagagacaatagtactaatggcagttcattgcatgaattttaaaagaaggggaggaataggggatatgaccccatcagaaagactaatcaatatgatcaccacagagcaagaaatacagtttctccaagccaaaaattcaaaattacaaaattttcgggtctatttcagagaaggcagagatcagttgtggaaaggacctggggagctactgtggaagggagacggagcagtcatagtcaaggtaggaacagatataaaagtagtaccaagcaggaaagccaag

SEQ ID NO: 18 - DNA, 2955 bp, Human immunodeficiency virus type 2
aacagcacccccagtagatccagcagcggacctgctagaaaagtacctgcagcaagggagaaagcagagagagcagagagagagaccatacaaagaggtgacagaggacttgctacatctcgagcaaggagagacaccacacagggagacaacagaggacttgctgcacctcaattctctctttggaaaagaccagtagtcacagcacacattgagggccagcccgtagaagttttactagacacaggggctgatgactcaatagtagcaggaatagagttagggagcgattataatccaaaaatagtagggggaatagggggattcataaataccaaagaatataaaaatgtaaaaatagaagtactaaataaaaggataagagccactataatggtaggtgataccccaatcaatatttttggcagaaacattctgacagccttaggcatgtcattaaatttaccaattgccaagatagaaccaataaaagtaacactgaagccaggaaaagatgggccaaaactgagacaatggcccctaacaaaagaaaaaatagaagcactaaaagaaatttgtgagaaaatggaaagagaaggccagctagaggaagcacctccaactaacccttataatacccccacatttgcaattaagaaaaaggacaaaaacaaatggagaatgctaatagatttcagagaactgaacaaagtaactcaggacttcacagaaattcagttaggaattccacacccagcaggattagccaagaaaaggagaattactgtactagatgtaggggatgcctacttttccataccactatgtgaggactttagacaatatactgcgtttaccctaccatcagtaaacaatgcagaaccaggaaaaagatatatatataaagtcttaccacaaggatggaaggggtcaccagcaatttttcaatacacaatgaggcagatcttagaaccattcaggaaagcaaacccagatgtcattctcgttcagtacatggatgatatcttaatagctagtgacaggacagatttagaacatgatagagtggtcctgcagctaaaggagcttctaaatggcctagggttctccaccccagatgagaagttccagaaagaccccccataccgatggatgggctatgaactatggccaaccaaatggaagttgcaaaaaatacagttaccccaaaaagaggtgtggacagtcaatgacatccaaaagctagtaggtgtcctgaattgggcagcacaaatctacccagggataaagaccaaacacttatgtagactaattaggggaaaaatgacactcacagaagaagtgcagtggacagaactagcagaggcagagctagaagagaacaagattatcctaagtcaggaacaggagggacactattaccaagaagaaaaggagctagaagcaacagtccataaggaccaagacaatcagtggacatataaaatacaccagggagaaaaaattctgaaagtaggaaagtatgcaaaaataagaaatacccataccaacggggtcagactattagcccaggtagttcagaaaataggaaaagaagcattagtcatctggggacgaataccaaagtttcacctaccagtagaaagagaaacctgggaacagtggtgggatgactactggcaagtaacatggatcccagactgggacttcgtatctaccccaccactggtcaggttagcatttaacctagtaagagatcctatactaggcgcagagaccttctacacagatggatcctgcaataggcaatcaaaagaagggaaggcaggatatataacagatagagggagaaacaaggtaaagatgctagagcaaaccaccaatcagcaagcagaattagaagcctttgctatggcaataacagactcaggcccaaaagccaacatcatagtagactcacagtatgtaatggggataatagcaggccagccgacagaatcagagagtaaaatggtaaatcaaatcatagaagaaatgataaaaagagaagcaatctatgttgcatgggtcccagcccataaaggcataggaggaaatcaggaggtagatcatttagtaagtcagggcatcagacaagtactatttctagaaaaaatagagcccgctcaggaagaacatgaaaaatatcacagcaatgtaaaggaattatcccataaatttggattacccaaattagtggcaaggcagatagtaaatacatgtgcccaatgtcaacagaaaggggaggctatacatgggcaagtaaatgcagaattaggtgtctggcaaatggactgcacacacttagaaggaaagatcattatagtagcagtacatgttgcaagtgggtttatagaagcagaagtcatcccacaggaatcaggaagacaaacagcactcttcctattaaaactggctagcagatggccaataacacacttgcacacagataatggtgccaacttcacctcacaagaagtgaagatggtagcatggtgggtaggcatagaacaatcctttggagtaccttacaatccacaaagccaaggagtagtagaagcaatgaatcaccacctaaaagatcagataagcagaattagagagcaggcaaatacagtagaaacaatagtactaatggcagttcattgcatgaattttaaaagaaggggaggaataggggatatgaccccagcagaaagactaatcaatatgatcaccgcagaacaagaaatacaattcctccaagcaaaaaattcaaaattaaaaaattttcgggtctatttcagagaaggcagagatcagctgtggaaaggccctggggagctactgtggaagggagacggagcagtcatagtcaaggtaggggcagacataaaaataattccaagaaggaaggccaagattatcagagactat

SEQ ID NO: 19 - DNA, 2961 bp, Human immunodeficiency virus type 2
aacagcacccccagtagatccagcagtggacctactggagaaatatatgcagcaagggaaaagacagagagagcagagagagagaccatacaaagaggtgacagaggacctgctgcacctcgaggagggaaatacaccatgcagggagacgacagaggacttgctgcacctcaattctctctttggaaaagaccagtagtcacagcacacattgagggtcagccagtagaagttttactagacacaggggctgacgactcaatagtagcgggaatagagctaggatgcgattatagcccaaaaatagtaggaggaatagggggattcataaataccaaagaatataaagatgtagaaatagaagttctaggtagaagggtaagggccaccataatgacaggagataccccaatcaacatttttggtagaaatgttctgacagccttaggcatgtcattaaatctaccagttgccaaaatagaaccaataaaagtaatgttaaaaccaggcaaggatggaccaaaactgagacagtggcccttaacaaaagaaaagatagaagcactaaaagaaatctgtgaaaaaatggaaaaggaaggccaactagaagaagcacccccaactaatccctttaatacccccacatttgcaatcaggaaaaaggacaaaaacaaatggagaatgctaatagattttagagaactaaacaaggtaactcaagatttcacagaaattcagttaggaattccacacccagcaggactagccaaaaagagaaggattactgtactagatgtaggggatgcttacttttccataccactacatgaagactttagacagtatactgcatttactttaccatcagtgaacaatgcagaaccaggaaaaagatacatatataaagtcttgccacagggatggaagggatcaccagcaatttttcaatacacaatgagacatatcttagaaccattcagaaaagcaaaccaggatgtcattatcattcagtatatggatgatatcttaatagctagtgacaggacagatctagaacatgacagagtagtcctgcagctcaaggaacttctaaatggcctagggttttctaccccagatgaaaaattccaaaaagaccctccataccgatggatgggctatgagctatggccaactaaatggaagttgcagaaaatacagttgcctcaaaaagaagtatggacagtcaatgacatccaaaaactagtaggtgtgctaaattgggcagcgcaaatctacccagggataaagacaaaacacttatgtaggttaattaggggaaaattgacactcacagaggaagtactgtggacagagttagcagaagcagaactagaagaaaacagaattatcttgagccaggaacaagagggacactactaccaagaagatagggatttagaagcaacagtccaaaaagatcaagacaatcagtggacatataaaatacaccagggagagaaaattctaaaagtaggaaaatatgcaaaggtaaaaaatacccataccaatggagtcaggttgttagcacaggtagttcagaaaataggaaaagaagcactagtcatttggggacgaataccaaaatttcacctaccagtagaaagagaaacttgggaacagtggtgggataactattggcaggtgacatggatcccagactgggactttgtatctaccccaccactggtcaggttagcatttaacctggtaaaagatcctataccaggcacagagaccttctacacagatgggtcctgcaataggcaatcaaaggaaggaaaagcaggatatataacagatagagggagagacaaggtaagggtactagagcaaactaccaatcagcaagcagaattagaagcctttgcaatggccctaacagactcaggtccaaaagttaatattatagtagactcacagtatgtaatgggaatagtggcaggccaaccaacagagtcagaaagcaaaatagtaaatcaaatcatagaagaaatgataaaaaaggaagcactctatgttgcatgggtcccagcccacaaaggcatagggggaaatcaggaagtagatcatttagtgagtcagggcatcagacaagtattgttcctagaaagaatagagcccgctcaagaagaacatgaaaaatatcatagcaatataaaagaactatcccataaatttggattacccaaactagtggcaagacaaatagtaaacacatgtgcccaatgccaacagaaaggggaagctatacatgggcaagtagatgcagaactaggcacttggcaaatggactgcacgcatttagaaggaaaggtcattatagtagcagtacatgttgcaagtggatttatagaagcagaagtcatcccacaggaaacaggaaggcaaacagcactcttcctgctaaaactggccagcaggtggccaataacacacttgcacacagataatggtgccaacttcacttcacaggaagtaaagatggtagcatggtggataggtataaaacaatcctttggagtaccttacaatccacaaagccaaggggtagtagaagccatgaatcaccacctaaaaaatcagataagtagaatcagagaccaggcaaatacagtagaaacaatagtactaatggcagttcatagcttgaattttaaaagaaggggaggaataggggatatgaccccatcagaaagattaatcaatatgatcaccacagaacaagaaatacaattcctccaagcaaaaaattcaaaattacaaaattttcgggtctatttcagagaaggcagagatcagttgtggaaaggacctggggagctactgtggaagggagacggagcagtcatagtcaaggtaggaacagacataaaagtagtaccaaggagaaaagccaagatcatcagagactatggagga

SEQ ID NO: 20 - DNA, 2955 bp, Human immunodeficiency virus type 2
aacagcacccccagtggatccagcagtagacctgctggaggaatatatgcagcagggcaagaggctgagagagcagagagagagaccatacaaggaagtgacagaggacctgctgcacctcgagcagggagagacaccgcacagggagacaacagaggacttgctgtgcctcaattctctctttggaaaagaccagtagtcacagcacacattgagggccagccagtagaagttttactagacacaggggctgacgactcaatagtagcaggaatagagttagggagcgattatagtccaaaaatagtagggggaataggaggattcataaataccaaagaatacaaaaatgtagaaataagagtactaaataaaagggtgagagccaccataatgacaggtgataccccaatcaacatttttggcagaaatattctgacagccttaggcatgtcattgaatctaccagtcgccaagatacaaccaataaaagtaatgctaaaaccaggaaaagatggaccaaaactgaggcagtggcccttaacaaaagagaaaatagaagcactaaaagaaatctgtgaaaaaatggaaagagaaggccagatagaggaagcatctccaactaatccttataatacccctacatttgcaattaagaagaaggacaaaaacaaatggagaatgctaatagattttagagaactaaacaaggtaactcaagagttcacagaaattcagttaggaattccacacccagcaggattagccaagaaaagaagaattactgtactagatataggggatgcttactttttcatacccctacatgaggattttagacagtatactgcatttactctaccatcagtgaacaatgcagagccaggaaaaagatatatatacaaggttttaccacagggatggaagggatcaccagcaatttttcaatacacaatgaggcagattttagaacccttcagaaaagcaaacccagatgtcatcctcattcagtacatggatgatatcttaatagctagcgacagaacagatttggaacatgacagagtagtcctgcagttaaaggaacttctaaacggcctaggattttccaccccagatgagaagttccaaaaagaccccccataccaatggatgggctatgaactgtggccgactaaatggaagctgcaaaaaatacaattgccccagaaagaaatatggacagtcaatgacatccaaaagctagtgggtgtcctaaattgggcagcgcaaatctacccagggataaagaccagacacctatgcagactaattagaggaaaaatgacactcacagaagaggtacagtggacagaactggcagaagcagagctagaagagaacaaaattattttaagccaggaacaagagggatgctattaccaagaagaaaaggagttagaggcaacagtccaaaaggaccaagacaatcagtggacatataagatacaccagggagaaaagattctaaaagtaggaaaatatgcaaaagtaaaaaatacccacaccaacggggtcagattgttagcacaggtagtccaaaaaataggaaaagaagcactagtcatttggggacgaataccgaaatttcacctaccagtagaaagagagacctgggaacagtggtgggataactattggcaagtaacatggattcccgactgggacttcgtatccaccccaccactggttagattagcatttaacctggtaaaagatcctataccaggcgcagagaccttctacacagatggatcctgcaatagacaatcaaaagaaggaaaagcaggatacataacagatagagggagagacaaggtgagaatactagagcagattaccaatcaacaagcagaattagaagcctttgcaatggcaataacagactcaggcccaaaagccaatattatagtagattcacagtatgtaatgggaatagtagcaggccagccaacagaatcagagagtagaatagtaaaccaaatcatagagggaatgataaaaaaggaagcaatctatgttgcatgggtcccagcccataaaggcatagggggaaatcaggaagtagatcacttagtaagtcagggcatcagacaagtattatttctagagaaaatagagcccgctcaggaagaacatgagaaatatcatagcaatgtaaaagaactatcccataaatttggcttacctaagctagtagcaagacagatagtaaacacatgtgcccaatgtcaacagaaaggggaggctatacatgggcaagtgaatgcagaattgggcacttggcaaatggactgcacacacttagaagggaaaatcatcatagtagcagtacatgttgcaagtggatttatagaagcagaagtcatcccacaggaaacaggaagacaaacagcactcttcctattaaaactagctagtagatggccaataacacacttgcacacagataatggtgccaacttcacctcacaggaggtgaagatggtagcatggtgggtaggtatagagcagacctttggagtaccttataatccacaaagccaaggagtagtagaggcaatgaaccaccacctaaagaatcagataagtaaaattagagaacaggcaaatacaatggagacaatagtattaatggcaacacattgcatgaattttaaaagaaggggaggaataggggatatgaccccagcagaaagactaatcaatatgatcaccacagaacaagagatacagttcctccacgcaaaaaatttaaaattaaaaaattttcgggtctatttcagagaaggcagagatcagctgtggaaaggacctggggaactactgtggaagggggacggagcagtcatagtcaaggtagggacagacataaaaatagtaccaagaaggaacgcgaagatcatcaccgactat

SEQ ID NO: 21 - DNA, 3030 bp, Human immunodeficiency virus type 2
aaagaagcctcgcaacttccccatggcccaaatgcctcagggagtgacaccatctgcacccccgatgaacccagcagaggacatgacacctcagagggcgacaccatctgcgccccctgcagatccagcagtggagatgctaaagagttacatgcagatggggagacaacagagagagagaccctacaaggaggtgacagaagatttgctgcacctcaattctctctttggagaagaccagtagttagggcatgtatcgagggtcagccagtagaagtattactagacacaggggctgacgactcaatagtagcaggaatagaattaggtagcaattacaccccaaaaatagtaggagggataggagggttcataaataccaaagaatacaaagatgtagaaatagaagtagtgggaaaaagggtaagggcaactataatgacaggagacaccccaataaacatttttggcagaaatattttaagtactttgggcatgactctaaatttcccagtagcaaaagtagaaccagtaaaagttgaactgaaacctggaaaagatggaccaaggatcagacaatggcctctatccagggaaaagatactagccctcaaagaaatctgtgaaaaaatggaaaaggaggggcaattagaagaagcaccccctaccaatccatacaacacacccaccttcgctgtaagaaagagagataaaaacaaatggagaatgctaatagactttagagaattaaacaaggttacccaggacgtcacagaagtccaattgggtattccccacccggcagggttggcagaaaaaaggaggataacagtattagatgtgggagatgcctacttcagtatcccactagacccaaacttcagacagtatacggcattcaccttgccatcaataaacaatgcagagccaggaaagagatacatttataaagttctaccacaaggatggaaggggtccccagcaatctttcaatactccatgaggaaggtattagatcctttcagaaaggccaacagtgatgtcattataattcagtacatggatgacatccttatagcaagtgacagaagtgatctggagcatgacaaggtagtgtcccaactaaaagaactattaaatgacatgggattctctaccccagaagaaaagttccaaaaagaccctccgttcaaatggatgggttatgagctctggccaaaaaagtggaaactgcaaaaaatacaactgccagaaagggaaatttggacagtaaatgacattcaaaaactggtgggagtgttaaactgggcagctcaactcttccctggaattaagacaaggcacatatgtaaactaattaggggaaagatgaccctaacagaagaagtacagtggacagaattagcggaagcagagttgcaggaaaataaaatcatcttagaacaagaacaagaaggatcctactacaaggaagggataccgctagaagcaacagtacagaaaaacctagcaaatcagtggacatacaaaattcatcagggagagagaattttaaaagtaggaaaatatgcaaaggttaaaaacacccacaccaacggagtaagactactggcacatgtagttcagaagataggaaaagaagccctagtcatctggggagagataccagtattccatctgccagtagaaagagagacgtgggaccagtggtggacagattactggcaagtaacctggatcccagagtgggactttgtctcgaccccaccattaataagactagcctacaacctagtcaaagaccccctagaagggagagaaacctactacacagatgggtcctgcaataaaacctcaaaagagggaaaagcaggatatgtcacagacaggggaaaagataaagttaaaatgttagaacagacaacaaaccaacaagcagaacttgaagcgtttgcattagcattacaggattcaggaccacaagttaacatcatagtagattcacaatatgtcatgggaataatagctgcacagccaacagaaacagaatcaccaatagtaacaagaataattaaagaaatgatcaaaaaggaggcaatatatgtaggatgggtaccagcccacaagggactaggtggtaatcaggaagtagaccacctagtgagtcagggaatcagacaggtcttgttcctagaaaaaatagaaccagcccaagaagagcatgaaaaatatcatggcaatgtaaaagacctggtccataaattcggaatcccacaattagtggcaaaacagatagtaaattcctgtgacaaatgccaacaaaaaggagaagctgttcatggacaggtaaatgcagatctagggacatggcagatggactgtacacatttagaaggaaagattataatagtggcagtccatgtggccagtgggtttatagaagcagaagtaataccccaagaaacaggaagacagacagctctcttcctattaaagttggccagcagatggcctatcacacacctgcatacagacaacggtgccaacttcacctcacaagatgtgaagatggtagcctggtgggtaggaatagaacaaacctttggagtaccctataacccacaaagtcaaggagtagtagaagcaatgaaccatcacctaaaaaatcaaatagacagactcagagaccaagcagtatcaatagagacagttgtgctaatggcaactcactgcatgaattttaaaagaaggggaggaataggggatatgacccctgcagaaagactagttaacatgataaccacagagcaagaaatacagttcctccaagcaaaaaatttaaaattccaaaatttccaggtctattacagagaaggcagagatcaactctggaagggacctggtgaactcttgtggaaaggggaaggagcagtcatcataaaggtagggacagaaatcaaagtagtacccaggagaaaagcaaaaattataaggcactatggaggaggaaaagaattggattgtagtgcc

SEQ ID NO: 22 - DNA, 3075 bp, Human immunodeficiency virus type 2
aaagaagcctcgcaacttccctgtaacccaagcaccccaggggataatgccatctgcacccccaatgaacccagcagtgaacatgacgcctcagggtgcgatgccatctgcgccccctgtagacccagcagaggagatgctgaagaactacatgcaactagggaaaaagcagaaggagagccgagagagaccctacaaggaggtgacagaggatttgctgcacctcaattctctctttggagaagaccagtagtcaaagcaaatatcgagggtcagccagtagaagtgttactagacacaggggctgatgactcaatagtagcagggatagaattaggcaacaattacaccccaaaaatagtaggagggataggaggatttataaataccaaagaatacaaaaatgtagaagtagaagtagtaggaaaaagagtaagagcaacaataatgacaggggacaccccaataaatatttttggcagaaatattctaaatagcctaggcatgactctaaatttcccagtagcaagcatagaaccagtaaaagtcaagctaaaagaaggaaaggatgggccaaaaatcagacaatggcccctatctaaagagaaaatacaggccctcaaagaaatctgtgagaaaatggagaaggagggacaattagaagaagcgcctcctactaatccatacaattcgcccacccttgccataaaaaagagagacaaaaacaaatggaggatgctaatagatttcagggaactaaacaaggtaacccaggaatttacagaagtccagctgggtattccccacccagcaggactggcatctaagaaaagaataacagtactagatgtaggagatgcctatttcagtatcccactggatccagacttcagacaatatacagcatttactttgccatcagtaaataatgcagaaccaggaaagagatatctttacaaagtcctaccacaagggtggaaaggatccccagcaattttccaatactccatgggaaaggtactagaccccttcagaaaagccaacagtgatgtcactataatccagtacatggatgacatccttgtggcaagtgacaggagcgatctagagcatgacaaggtagtgtctcagctaaaagaactattaaataacatgggattctctaccccagaagaaaagttccaaaaggaccctccattccaatggatgggatatgagctctggccaaagaagtggaaactgcagaaaatacaactgccagaaaaagaggtttggacagtaaatgacattcagaaattagtaggagtattaaattgggcagctcaacttttcccgggaattaagacaaggcacatatgtagactaatcaggggaaaaatgaccctaacagaagaggtacaatggacagaattagcagaggcagaattagaggaaaacaaaatcatcctagaacaagagcaagaagggtcctattacaaagaaggggtacctttagaagcaacagtgcagaaaaatctagcaaatcagtggacatacaagattcatcagggagataaaatcctaaaagtaggaaaatatgcaaaggtcaaaaacactcacaccaatggagtaagactactagctcatgtagtccaaaaaataggaaaggaggcattagtcatttggggagaagtaccaatatttcatctaccagtagaaagagagacatgggatcagtggtggacagattactggcaagtaacctggattccagaatgggattttgtctcaaccccaccattaataaggttagcctataacctggtcaaagaccccctggaaggagtagaaacttactacacagatggatcctgtaacagaacctcaaaagaaggaaaggcaggatatgtcacagacaggggaaaagataaagttaaaccattagaacaaacaacaaatcagcaagcagaacttgaagcatttgcattagcactacaggattcaggaccacaggtcaatatcatagtagattcacaatatgtcatggggataatagctgcacagccaacagaaacagaatcaccgatagtaaaaacaataattgaagaaatgatcaaaaaagaagcaatatatgtaggatgggtaccagctcacaaaggactgggtggtaatcaggaagtagaccacctagtaagtcaaggaattagacagatcctgtttctagaaaatatagaaccagcccaagaagaacatgaaaaatatcatggtaatataaaagaactggtccataaattcggaatcccacaattagtggcaagacaaatagtaaattcctgtaataaatgccaacaaaaaggggaggctattcatggacaggtaaattcagaactagggacatggcaaatggactgtacacatttagagggaaaggttataatagtggcagtccatgtagccagtggattcatagaagcagaggtaataccccaagaaacaggaagacagacagctctcttcctgttaaagctggccagcagatggcctatcacacacctacacacagacaacggtgccaacttcacttcacaagatgtgaagatggtagcctggtgggtaggaatagaacaaacctttggagtaccctataatccacaaagtcagggagtagtagaatcaatgaaccatcatctaaaaaatcaaatagacagaatcagagaccaggcagtatcaatagagacagttgtgctaatggcagctcactgcatgaattttaaaagaaggggaggaataggggatatgacccctgcagaaagaatagtcaacatgataaccacagaacaggaaatacaattcctccaaacaaaaaatttaaaattccaaaatttccgggtctattacagagaaggcagagatcaactctggaagggacccggtgaactattgtggaaaggggaaggagcagtcatcataaaggtagggacagaaatcaaagtaatacccagaagaaaagcaaagatcataaggaactatggaggaggaaaagacttggattgtagtgccgacatggaggataccaggcaggctagagaagtggca

SEQ ID NO: 23 - DNA, 21 bp, Artificial Sequence
  Synthetic oligonucleotide primer
ccttacaatccacaaagccaa

SEQ ID NO: 24 - DNA, 23 bp, Artificial Sequence
  Synthetic oligonucleotide primer
attgtatttcttgttctgtggtg

SEQ ID NO: 25 - DNA, 50 bp, Artificial Sequence
  Synthetic oligonucleotide primer
ctgtatttgcctgytctctaattcttttttagtagaagcaatgaatcacc

SEQ ID NO: 26 - DNA, 48 bp, Artificial Sequence
  Synthetic oligonucleotide primer
agtactaatggcagttcattgcatgttttgtctttctgctggggtcat

SEQ ID NO: 27 - DNA, 18 bp, Artificial Sequence
  Synthetic oligonucleotide primer
acttatctgattttttag

SEQ ID NO: 28 - DNA, 21 bp, Artificial Sequence
  Synthetic oligonucleotide primer
aattttaaaagaaggggagga

SEQ ID NO: 29 - DNA, 21 bp, Artificial Sequence
  Synthetic oligonucleotide primer
ccctataacccacaaagtcag

SEQ ID NO: 30 - DNA, 23 bp, Artificial Sequence
  Synthetic oligonucleotide primer
attgtatttcttgttctgtggtt

SEQ ID NO: 31 - DNA, 50 bp, Artificial Sequence
  Synthetic oligonucleotide primer
ttgatactgcctgrtctctgattcttttttagtagaagcaatgaaccatc

SEQ ID NO: 32 - DNA, 48 bp, Artificial Sequence
  Synthetic oligonucleotide primer
tgtactaatggcagctcactgcatgttttgtctttctgcaggggtcat

SEQ ID NO: 33 - DNA, 18 bp, Artificial Sequence
  Synthetic oligonucleotide primer
gtctatttgattttttag

SEQ ID NO: 34 - DNA, 21 bp, Artificial Sequence
  Synthetic oligonucleotide primer
aattttaaaagaaggggagga

SEQ ID NO: 35 - DNA, 15 bp, Artificial Sequence
  Synthetic oligonucleotide primer
ccttcttttaaaatt

SEQ ID NO: 36 - DNA, 23 bp, Artificial Sequence
  Synthetic oligonucleotide primer
tggaagggatgttttacagtgag

SEQ ID NO: 37 - DNA, 26 bp, Artificial Sequence
  Synthetic oligonucleotide primer
tggaagggatttactatagtgagaga

SEQ ID NO: 38 - DNA, 30 bp, Artificial Sequence
  Synthetic oligonucleotide primer
tggaagggattttttatagtgaaagaagac

SEQ ID NO: 39 - DNA, 20 bp, Artificial Sequence
  Synthetic oligonucleotide primer
ggattttcctgccttggttt

SEQ ID NO: 40 - DNA, 17 bp, Artificial Sequence
  Synthetic oligonucleotide primer
tcccgctcctcacgctg

SEQ ID NO: 41 - DNA, 22 bp, Artificial Sequence
  Synthetic oligonucleotide primer
caggaaaatccctagcaggttg

SEQ ID NO: 42 - DNA, 27 bp, Artificial Sequence
  Synthetic oligonucleotide primer
tgctagggattttcctgcctccgtttc

SEQ ID NO: 43 - DNA, 22 bp, Artificial Sequence
  Synthetic oligonucleotide primer
caacctgctagggattttcctg

SEQ ID NO: 44 - DNA, 20 bp, Artificial Sequence
  Synthetic oligonucleotide primer
caacagcacccccagtagat

SEQ ID NO: 45 - DNA, 20 bp, Artificial Sequence
  Synthetic oligonucleotide primer
ggaaagaagcctcgcaactt

SEQ ID NO: 46 - DNA, 24 bp, Artificial Sequence
  Synthetic oligonucleotide primer
agccaagcaatgcagggctcctag

SEQ ID NO: 47 - DNA, 20 bp, Artificial Sequence
  Synthetic oligonucleotide primer
atcttggctttcctrcttgg

SEQ ID NO: 48 - DNA, 20 bp, Artificial Sequence
  Synthetic oligonucleotide primer
ggcactacaatccaattctt

SEQ ID NO: 49 - DNA, 20 bp, Artificial Sequence
  Synthetic oligonucleotide primer
tgcaagtccaccaagcccat

SEQ ID NO: 50 - DNA, 27 bp, Artificial Sequence
  Synthetic oligonucleotide primer
atagtcrrtgatgatcttygcrttcct

SEQ ID NO: 51 - DNA, 20 bp, Artificial Sequence
  Synthetic oligonucleotide primer
ccaagtgggaaccactatcc

SEQ ID NO: 52 - DNA, 30 bp, Artificial Sequence
  Synthetic oligonucleotide primer
gttgcaattctcctgttctatgcttcagat

SEQ ID NO: 53 - DNA, 21 bp, Artificial Sequence
  Synthetic oligonucleotide primer
cggagaggctggcagatygag

SEQ ID NO: 54 - DNA, 21 bp, Artificial Sequence
  Synthetic oligonucleotide primer
ggcagaggctggcagattgag

SEQ ID NO: 55 - DNA, 22 bp, Artificial Sequence
  Synthetic oligonucleotide probe
tccagcactagcaggtagagcc

SEQ ID NO: 56 - DNA, 24 bp, Artificial Sequence
  Synthetic oligonucleotide probe
ctccagcactarcaggtagagcct

SEQ ID NO: 57 - DNA, 23 bp, Artificial Sequence
  Synthetic oligonucleotide primer
ggtgagagtcyagcagggaacac

SEQ ID NO: 58 - DNA, 26 bp, Artificial Sequence
  Synthetic oligonucleotide primer
gtgtgtgttcccatctctcctagtcg

SEQ ID NO: 59 - DNA, 26 bp, Artificial Sequence
  Synthetic oligonucleotide primer
gtgtgtgytcccatctctcctagtcg

SEQ ID NO: 60 - DNA, 20 bp, Artificial Sequence
  Synthetic oligonucleotide probe
acaccgartgaccaggcggc

SEQ ID NO: 61 - DNA, 22 bp, Artificial Sequence
  Synthetic oligonucleotide probe
ccgcctggtcatycggtgttca

SEQ ID NO: 62 - DNA, 22 bp, Artificial Sequence
  Synthetic oligonucleotide probe
ccgcctggtcattcggtgctcc

SEQ ID NO: 63 - DNA, 25 bp, Artificial Sequence
  Synthetic oligonucleotide primer
gcagaaagggtcctaacagaccagg

SEQ ID NO: 64 - DNA, 26 bp, Artificial Sequence
  Synthetic oligonucleotide primer
gcragaagggtcctaacagaccaggs

SEQ ID NO: 65 - DNA, 25 bp, Artificial Sequence
  Synthetic oligonucleotide primer
caccacacagagaggcgacagagga

SEQ ID NO: 66 - DNA, 25 bp, Artificial Sequence
  Synthetic oligonucleotide primer
caccatgcagggaracgacagagga

SEQ ID NO: 67 - DNA, 25 bp, Artificial Sequence
  Synthetic oligonucleotide primer
gaccctacaaggaggtgacrgagga

SEQ ID NO: 68 - DNA, 25 bp, Artificial Sequence
  Synthetic oligonucleotide probe
tgctgcacctcaattctctctttgg

SEQ ID NO: 69 - DNA, 25 bp, Artificial Sequence
  Synthetic oligonucleotide probe
tgctgtgcctcaattctctctttgg

SEQ ID NO: 70 - DNA, 31 bp, Artificial Sequence
  Synthetic oligonucleotide primer
tgaccctcratgtrtgctgtgactactggtc

SEQ ID NO: 71 - DNA, 31 bp, Artificial Sequence
  Synthetic oligonucleotide primer
tgaccctcratacatgctttgactactggtc

SEQ ID NO: 72 - DNA, 31 bp, Artificial Sequence
  Synthetic oligonucleotide primer
tgaccctcgatatatgcttggactactggtc

SEQ ID NO: 73 - DNA, 31 bp, Artificial Sequence
  Synthetic oligonucleotide primer
taatggcagytcaytgcatgaattttaaaag

SEQ ID NO: 74 - DNA, 31 bp, Artificial Sequence
  Synthetic oligonucleotide primer
tratggcaacwcactgcatgaattttaaaag

SEQ ID NO: 75 - DNA, 30 bp, Artificial Sequence
  Synthetic oligonucleotide primer
agtaytaatggcagttcaytgcatgaattt

SEQ ID NO: 76 - DNA, 30 bp, Artificial Sequence
  Synthetic oligonucleotide primer
tgtactaatggcagctcaytgcatgaattt

SEQ ID NO: 77 - DNA, 24 bp, Artificial Sequence
  Synthetic oligonucleotide probe
tcatatcccctattcctccccttc

SEQ ID NO: 78 - DNA, 26 bp, Artificial Sequence
  Synthetic oligonucleotide probe
aggggaggaataggggatatgacycc

SEQ ID NO: 79 - DNA, 31 bp, Artificial Sequence
  Synthetic oligonucleotide primer
ggaggaattgtatytcttgttctgtggtrat

SEQ ID NO: 80 - DNA, 31 bp, Artificial Sequence
  Synthetic oligonucleotide primer
ggargaattgtatttcttgttctgtrgttat

SEQ ID NO: 81 - DNA, 31 bp, Artificial Sequence
  Synthetic oligonucleotide primer
ggaagaactgtatttcttgctctgtggttat

SEQ ID NO: 82 - DNA, 34 bp, Artificial Sequence
  Synthetic oligonucleotide primer
ggaggaattgtatttcttgttctgtggtnatcat

SEQ ID NO: 83 - DNA, 34 bp, Artificial Sequence
  Synthetic oligonucleotide primer
ggaagaattgtatttcttgytctgtggttatcat

SEQ ID NO: 84 - DNA, 21 bp, Artificial Sequence
  Synthetic oligonucleotide primer
ctcggactttaytggccggga

SEQ ID NO: 85 - DNA, 21 bp, Artificial Sequence
  Synthetic oligonucleotide primer
cccggactttaytggctggga

SEQ ID NO: 86 - DNA, 22 bp, Artificial Sequence
  Synthetic oligonucleotide probe
agtgcagcarcagcaacagctg

SEQ ID NO: 87 - DNA, 22 bp, Artificial Sequence
  Synthetic oligonucleotide primer
ccccagacggtcagycgcaaca

SEQ ID NO: 88 - DNA, 22 bp, Artificial Sequence
  Synthetic oligonucleotide primer
ccccagacggtcaatctcaaca

SEQ ID NO: 89 - DNA, 19 bp, Artificial Sequence
  Synthetic oligonucleotide primer
ttggcgccygaacagggac

SEQ ID NO: 90 - DNA, 21 bp, Artificial Sequence
  Synthetic oligonucleotide probe
agtgarggcagtaagggcggc

SEQ ID NO: 91 - DNA, 23 bp, Artificial Sequence
  Synthetic oligonucleotide primer
gcactccgtcgtggtttgttcct

SEQ ID NO: 92 - DNA, 23 bp, Artificial Sequence
  Synthetic oligonucleotide primer
gcwctccgtcgtggttgattcct

SEQ ID NO: 93 - DNA, 23 bp, Artificial Sequence
  Synthetic oligonucleotide primer
gtgtttgcagatttggacctgcg

SEQ ID NO: 94 - DNA, 20 bp, Artificial Sequence
  Synthetic oligonucleotide probe
tgacctgaaggctctgcgcg

SEQ ID NO: 95 - DNA, 20 bp, Artificial Sequence
  Synthetic oligonucleotide primer
aggtgagcggctgtctccac

(19) United States
(12) Patent Application Publication
(10) Pub. No.: US 2017/0058366 A1
(43) Pub. Date: Mar. 2, 2017

(54) HIV-2 NUCLEIC ACIDS AND METHODS OF DETECTION

(71) Applicant: THE UNITED STATES OF AMERICA, AS REPRESENTED BY THE SECRETARY, DEPARTMENT OF HEALTH AND HUMAN SERV, Bethesda, MD (US)

(72) Inventors: Kelly A. Curtis, Atlanta, GA (US); Ae S. Youngpairoj, Atlanta, GA (US); Sherry M. Owen, Douglasville, GA (US); Chou-Pong Pau, Atlanta, GA (US); Timothy C. Granade, Conyers, GA (US); Philip Niedzwiedz, Atlanta, GA (US); Donna L. Rudolph, Lilburn, GA (US)

(73) Assignee: The United States of America, as represented by th e Secretary, Dept. of Health and Human Services, Bethesda, MD (US)

(21) Appl. No.: 15/120,270

(22) PCT Filed: Feb. 20, 2015
(86) PCT No.: PCT/US2015/016814

§ 371 Date: Aug. 19, 2016

Related U.S. Application Data
(60) Provisional application No. 61/943,001, filed on Feb. 21, 2014.

(51) Int. Cl. C12Q 1/70; C07K 14/005

(52) U.S. Cl. CPC C12Q 1/703; C07K 14/005; C12Q 2600/158; C12Q 2600/16; C12N 2740/16022

(57) ABSTRACT

Disclosed herein are methods of detecting HIV-2 nucleic acids in a sample (such as from a sample infected with or suspected to be infected with HIV-2). In some examples, the methods include LAMP or RT-LAMP, while in other examples, the methods include hybridization of a probe to an HIV-2 nucleic acid, including, but not limited to real-time PCR. Sets of LAMP primers for detection of HIV-2 Group A and Group B nucleic acids are provided herein. Sets of probes and primers for real-time PCR detection of HIV-2 nucleic acids are also provided herein. Finally, primers for amplification of HIV-2 nucleic acids are provided. Also disclosed are isolated HIV-2 nucleic acids, vectors including the HIV-2 nucleic acids, and cells transformed with vectors including HIV-2 nucleic acids.

### CROSS REFERENCE TO RELATED APPLICATION

This claims the benefit of U.S. Provisional Application No. 61/943,001, filed Feb. 21, 2014, which is incorporated herein by reference in its entirety

### FIELD

This disclosure relates to human immunodeficiency virus-2 (HIV-2) nucleic acids and methods of amplifying or detecting HIV-2 nucleic acids.

### BACKGROUND

HIV-2 emerged in West Africa and is closely related to simian immunodeficiency virus (SW) from sooty mangabeys. Although HIV-2 infections are primarily endemic to West Africa and in countries with socio-economic ties to West Africa, the virus has spread to geographically diverse countries due to international travel and migration. The majority of cases diagnosed outside this region have been in Portugal and France, with sporadic cases reported in other parts of Europe, North America, and Asia. At present eight distinct HIV-2 groups have been identified (HIV-2 A-H); however, groups C—H have only been identified in single isolated cases (Gao et al., J. Virol. 68:7433-7447, 1994; Chen et al., J. Virol. 71:3953-3960, 1997; Yamaguchi et al., AIDS Res. Hum. Retrovir. 16:925-930, 2000; Damond et al., AIDS Res. Hum Retrovir. 20:666-672, 2004).

Like HIV-1, HIV-2 infection can result in disease in humans (such as acquired immunodeficiency syndrome (AIDS)). Although HIV-2 is less pathogenic than HIV-1, accurate differentiation of HIV-1/2 is important due to the clinical implications of disease progression and for selection of appropriate treatment regimens, particularly because HIV-2 is intrinsically resistant to some non-nucleoside reverse transcriptase inhibitors and protease inhibitors used to treat HIV-1 infection (Campbell-Yesufu et al., Clin. Infect. Dis. 52:780-787, 2011; Camacho Intervirology 55:179-183, 2012). In addition, HIV-2 plasma viral loads are approximately 30-fold lower than those found in HIV-1 infections (Andersson et al., Arch. Intern. Med. 160:3286-3293, 2000). Thus, there remains a need for rapid, specific, and sensitive assays for HIV-2, particularly nucleic acid amplification tests.

### SUMMARY

Disclosed herein are methods of detecting HIV-2 nucleic acids in a sample (such as from a sample containing or suspected to contain HIV-2 nucleic acid). In some examples, the methods include loop-mediated isothermal amplification (LAMP) or reverse transcription-LAMP (RT-LAMP), while in other examples, the methods include hybridization of a probe to an HIV-2 nucleic acid, including, but not limited to real-time PCR. In some examples, the methods include contacting a sample with one or more sets of LAMP primers specific for HIV-2 under conditions sufficient to produce an amplification product and detecting the amplification product. In other examples, the methods include contacting a sample with a probe (such as a detectably labeled probe) capable of hybridizing to an HIV-2 nucleic acid and detecting the probe. The methods optionally include amplifying the HIV-2 nucleic acid before or concurrently with contacting the sample with the probe.

Sets of LAMP primers for detection of HIV-2 Group A nucleic acids (such as SEQ ID NOs: 23-28) and HIV-2 Group B nucleic acids (such as SEQ ID NOs: 29-34) are provided herein. Sets of probes and primers for real-time PCR detection of HIV-2 nucleic acids (such as SEQ ID NOs: 53-92) are also provided herein. Finally, primers for amplification of HIV-2 nucleic acids (such as SEQ ID NOs: 36-52) are provided.

Also disclosed herein are HIV-2 nucleic acids, for example isolated HIV-2 long terminal repeat (LTR) nucleic acids (such as SEQ ID NOs: 1-11) and isolated HIV-2 polymerase (pol) nucleic acids (such as SEQ ID NOs: 12-22). In some examples, the HIV-2 nucleic acids are incorporated into a vector, such as a recombinant bacterial, viral, yeast, or mammalian vector. Also disclosed are cells transformed with a recombinant vector that includes one or more HIV-2 nucleic acids.

The foregoing and other features of the disclosure will become more apparent from the following detailed description, which proceeds with reference to the accompanying figures.

### BRIEF DESCRIPTION OF THE DRAWINGS

FIGS. 1A and 1B show real-time detection of HIV-2 RNA by RT-LAMP. FIG. 1A is a graph showing the fluorescence intensity of the PicoGreen dye (mV) over time (minutes) for the HIV-2 NIH-Z RNA linearity panel. Sample concentrations (RNA copies/mL) of each panel member are shown. FIG. 1B is a digital image showing confirmation of amplification by agarose gel electrophoresis. Lanes 1-7 represent reagent control, 10⁶/mL, 10⁵/mL, 10⁴/mL, 10³/mL, 10²/mL, and negative control, respectively. M=molecular marker.

FIG. 2 is a digital image of reaction tubes of the multiplexed HW-1/2 reaction under ultraviolet (UV) light. The specific targets that were added to the reaction are indicated under each tube.

FIGS. 3A and 3B are diagrams showing the phylogenetic relationships of HIV-2 plasmid clones to previously characterized HIV-2 strains in the LTR (FIG. 3A) and pol (FIG. 3B) regions. The HIV-2 plasmid clones are shown in bold. The non-bold identifiers are references from the Los Alamos HIV/SIV Sequence Database including the outgroups (X14307SIV-LTR and AB253736SIV-pol), accession numbers X14307 and AB253736 for LTR and pol region, respectively. The trees were inferred by the Neighbor-Joining method and the numbers on branches are percent posterior probabilities (values of 99% and above are shown). The scale bars indicate 0.02 substitutions per site for LTR region and 0.05 for the Pol region.

FIGS. 4A-4D are plots showing sensitivity of real-time PCR assays for the detection of HIV-2 plasmid DNA. Primer/probe sets LTR1 (FIG. 4A) and LTR2 (FIG. 4B) were developed for the detection of LTR plasmids and the protease (Pro) (FIG. 4C) and integrase (Int) (FIG. 4D) primer/probe sets were developed for the detection of pol plasmids.

### SEQUENCE LISTING

Any nucleic acid and amino acid sequences listed herein or in the accompanying sequence listing are shown using standard letter abbreviations for nucleotide bases and amino acids, as defined in 37 C.F.R. §1.822. In at least some cases, only one strand of each nucleic acid sequence is shown, but the complementary strand is understood as included by any reference to the displayed strand.

SEQ ID NOs: 1-11 are HIV-2 LTR nucleic acid sequences SEQ ID NOs: 12-22 are HIV-2 pol nucleic acid sequences.

SEQ ID NOs: 23-28 are nucleic acid sequences of exemplary HIV-2 Group A LAMP primers.

SEQ ID NOs: 29-34 are nucleic acid sequences of exemplary HIV-2 Group B LAMP primers.

SEQ ID NO: 35 is the nucleic acid sequence of an exemplary HIV-2 LAMP quencher oligonucleotide.

SEQ ID NOs: 36-43 are nucleic acid sequences of exemplary HIV-2 LTR amplification primers.

SEQ ID NOs: 44-52 are nucleic acid sequences of exemplary HIV-2 pol amplification primers.

SEQ ID NOs: 53-64 are nucleic acid sequences of exemplary HIV-2 LTR real-time PCR primers and probes.

SEQ ID NOs: 65-72 are nucleic acid sequences of exemplary HIV-2 Pro real-time PCR primers and probes.

SEQ ID NOs: 73-83 are nucleic acid sequences of exemplary HIV-2 Int real-time PCR primers and probes.

SEQ ID NOs: 84-88 are nucleic acid sequences of exemplary HIV-2 Env real-time PCR primers and probes.

SEQ ID NOs: 89-92 are nucleic acid sequences of exemplary HIV-2 LTR-gag real-time PCR primers and probes.

SEQ ID NOs: 93-95 are nucleic acid sequences of exemplary RNase P real-time PCR primers and probes.

### DETAILED DESCRIPTION

### I. Terms

Unless otherwise noted, technical terms are used according to conventional usage. Definitions of common terms in molecular biology may be found in Lewin's Genes X, ed. Krebs et al, Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3rd Edition, Springer, 2008 (ISBN: 1402067534).

The following explanations of terms and methods are provided to better describe the present disclosure and to guide those of ordinary skill in the art to practice the present disclosure. The singular forms “a,” “an,” and “the” refer to one or more than one, unless the context clearly dictates otherwise. For example, the term “comprising a nucleic acid molecule” includes single or plural nucleic acid molecules and is considered equivalent to the phrase “comprising at least one nucleic acid molecule.” As used herein, “comprises” means “includes.” Thus, “comprising A or B,” means “including A, B, or A and B,” without excluding additional elements.

All publications, patent applications, patents, and other references mentioned herein are incorporated by reference in their entirety for all purposes. All sequences associated with the GenBank Accession Nos. of HIV Database Accession Nos. mentioned herein are incorporated by reference in their entirety as were present on Feb. 21, 2014, to the extent permissible by applicable rules and/or law. In case of conflict, the present specification, including explanations of terms, will control.

Although methods and materials similar or equivalent to those described herein can be used to practice or test the disclosed technology, suitable methods and materials are described below. The materials, methods, and examples are illustrative only and not intended to be limiting.

In order to facilitate review of the various embodiments of this disclosure, the following explanations of specific terms are provided:

Amplification: Increasing the number of copies of a nucleic acid molecule, such as a gene or fragment of a gene, for example at least a portion of an HIV nucleic acid molecule. The products of an amplification reaction are called amplification products. An example of in vitro amplification is the polymerase chain reaction (PCR), in which a sample (such as a biological sample from a subject) is contacted with a pair of oligonucleotide primers, under conditions that allow for hybridization of the primers to a nucleic acid molecule in the sample. The primers are extended under suitable conditions, dissociated from the template, and then re-annealed, extended, and dissociated to amplify the number of copies of the nucleic acid molecule. Other examples of in vitro amplification techniques include real-time PCR, quantitative real-time PCR (qPCR), reverse transcription PCR (RT-PCR), quantitative RT-PCR (qRT-PCR), loop-mediated isothermal amplification (LAMP; see Notomi et al., Nucl. Acids Res. 28:e63, 2000); reverse-transcriptase LAMP (RT-LAMP); strand displacement amplification (see U.S. Pat. No. 5,744,311); transcription-free isothermal amplification (see U.S. Pat. No. 6,033,881); repair chain reaction amplification (see International Patent Publication No. WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Conditions sufficient for: Any environment that permits the desired activity, for example, that permits specific binding or hybridization between two nucleic acid molecules or that permits reverse transcription or amplification of a nucleic acid. Such an environment may include, but is not limited to, particular incubation conditions (such as time and or temperature) or presence and/or concentration of particular factors, for example in a solution (such as buffer(s), salt(s), metal ion(s), detergent(s), nucleotide(s), enzyme(s), and so on).

Contact: Placement in direct physical association; for example in solid and/or liquid form. For example, contacting can occur in vitro with one or more primers and/or probes and a biological sample (such as a sample including nucleic acids) in solution.

Detectable label: A compound or composition that is conjugated directly or indirectly to another molecule (such as a nucleic acid molecule) to facilitate detection of that molecule. Specific, non-limiting examples of labels include fluorescent and fluorogenic moieties (e.g., fluorophores), chromogenic moieties, haptens (such as biotin, digoxigenin, and fluorescein), affinity tags, and radioactive isotopes (such as ³²P, ³³P, ³⁵S, and ¹²⁵I). The label can be directly detectable (e.g., optically detectable) or indirectly detectable (for example, via interaction with one or more additional molecules that are in turn detectable). Methods for labeling nucleic acids, and guidance in the choice of labels useful for various purposes, are discussed, e.g., in Sambrook and Russell, in Molecular Cloning: A Laboratory Manual, 3^{rd} Ed., Cold Spring Harbor Laboratory Press (2001) and Ausubel et al., in Current Protocols in Molecular Biology, Greene Publishing Associates and Wiley-Intersciences (1987, and including updates).

Fluorophore: A chemical compound, which when excited by exposure to a particular stimulus, such as a defined wavelength of light, emits light (fluoresces), for example at a different wavelength (such as a longer wavelength of light).

Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) eliminates the need for an external source of electromagnetic radiation, such as a laser.

Examples of particular fluorophores that can be used in the probes and primers disclosed herein are known to those of skill in the art and include 4-acetamido-4′-isothiocyanatostilbene-2,2′disulfonic acid; acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), QFITC (XRITC), 6-carboxy-fluorescein (HEX), and TET (tetramethyl fluorescein); fluorescamine; IR144; IR1446; Malachite Green isothiocyanate; 4-methylumbelliferone; ortho-cresolphthalein; nitrotyrosine; pararosaniline; Phenol Red; B-phycoerythrin; o-phthaldialdehyde; pyrene and derivatives such as pyrene, pyrene butyrate, and succinimidyl 1-pyrene butyrate; Reactive Red 4 (CIBACRON™ Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, N,N,N′,N′-tetramethyl-6-carboxyrhodamine (TAMRA), tetramethyl rhodamine, and tetramethyl rhodamine isothiocyanate (TRITC); sulforhodamine B; sulforhodamine 101 and sulfonyl chloride derivative of sulforhodamine 101 (Texas Red); riboflavin; rosolic acid and terbium chelate derivatives; LightCycler Red 640; Cy5.5; and Cy56-carboxyfluorescein; boron dipyrromethene difluoride (BODIPY); acridine; stilbene; Cy3; Cy5, VIC® (Applied Biosystems); LC Red 640; LC Red 705; and Yakima yellow amongst others. Additional examples of fluorophores include Quasar® 670, Quasar® 570, CalRed 590, CalRed 610, CalRed615, CalRed 635, CalGreen 520, CalGold 540, and CalOrange 560 (Biosearch Technologies, Novato, Calif.). One skilled in the art can select additional fluorophores, for example those available from Molecular Probes/Life Technologies (Carlsbad, Calif.).

In particular examples, a fluorophore is used as a donor fluorophore or as an acceptor fluorophore. “Acceptor fluorophores” are fluorophores which absorb energy from a donor fluorophore, for example in the range of about 400 to 900 nm (such as in the range of about 500 to 800 nm). Acceptor fluorophores generally absorb light at a wavelength which is usually at least 10 nm higher (such as at least 20 nm higher) than the maximum absorbance wavelength of the donor fluorophore, and have a fluorescence emission maximum at a wavelength ranging from about 400 to 900 nm. Acceptor fluorophores have an excitation spectrum that overlaps with the emission of the donor fluorophore, such that energy emitted by the donor can excite the acceptor. Ideally, an acceptor fluorophore is capable of being attached to a nucleic acid molecule.

In a particular example, an acceptor fluorophore is a dark quencher, such as Dabcyl, QSY7 (Molecular Probes), QSY33 (Molecular Probes), BLACK HOLE QUENCHERS™ (Biosearch Technologies; such as BHQ0, BHQ1, BHQ2, and BHQ3), ECLIPSE™ Dark Quencher (Epoch Biosciences), or IOWA BLACK™ (Integrated DNA Technologies). A quencher can reduce or quench the emission of a donor fluorophore.

“Donor Fluorophores” are fluorophores or luminescent molecules capable of transferring energy to an acceptor fluorophore, in some examples generating a detectable fluorescent signal from the acceptor. Donor fluorophores are generally compounds that absorb in the range of about 300 to 900 nm, for example about 350 to 800 nm. Donor fluorophores have a strong molar absorbance coefficient at the desired excitation wavelength, for example greater than about 10³ M⁻¹ cm⁻¹.

Isolated: An “isolated” biological component (such as a nucleic acid) has been substantially separated or purified away from other biological components that are present or in which the component naturally occurs, such as other chromosomal and extrachromosomal DNA, RNA, and proteins. Nucleic acids that have been “isolated” include nucleic acids purified by standard purification methods. The term also embraces nucleic acids prepared by recombinant expression in a host cell as well as chemically synthesized nucleic acid molecules. Isolated does not require absolute purity, and can include protein, peptide, or nucleic acid molecules that are at least 50% isolated, such as at least 75%, 80%, 90%, 95%, 98%, 99%, or even 99.9% isolated.

Human immunodeficiency virus (HIV): HIV is a retrovirus that causes immunosuppression in humans (HIV disease), and leads to disease states known as acquired immunodeficiency syndrome (AIDS) and AIDS related complex (ARC). “HIV disease” refers to a well-recognized constellation of signs and symptoms (including the development of opportunistic infections) in persons who are infected by an HIV virus, as determined by antibody or western blot studies or detection of HIV nucleic acids. Laboratory findings associated with this disease are a progressive decline in T cells. HIV includes HIV type 1 (HIV-1) and HIV type 2 (HIV-2). Related viruses that are used as animal models include simian immunodeficiency virus (SW) and feline immunodeficiency virus (FIV). HIV nucleic acid and protein sequences are available in public databases, including GenBank and the HIV Database (available on the World Wide Web at www.hiv.lanl.gov/). Exemplary reference sequences include HXB2 for HIV-1 (e.g., GenBank Accession Nos. K03455 or M38432) and MAC239 for HW-2 (GenBank Accession No. M33262).

The HIV genome contains three major genes, gag, pol, and env, which encode major structural proteins and essential enzymes. The gag gene encodes the Gag polyprotein, which is processed to six protein products. The pol gene encodes the Pol polyprotein, which is processed to produce reverse transcriptase (RT), RNase H, integrase (INT), and protease (PRO). Env encodes gp160, which is processed to the two envelope proteins, gp120 and gp41. In addition to these, HIV has two regulatory proteins (Tat and Rev) and accessory proteins (Nef, Vpr, Vif and Vpu). Each end of the HIV provirus has a repeated sequence referred to as a long terminal repeat (LTR).

HW-2 is genetically distinct from HIV-1. There are at least eight recognized groups of HIV-2 (Groups A-H). Groups A and B are responsible for the majority of cases of HIV-2 infection in human populations. The sequence diversity and epidemiology of HIV-2 viruses suggests that each of the individual HIV-2 groups may be the result of separate transmission occurrences from sooty mangabeys to humans (Santiago et al., J. Virol. 79:12515-12527, 2005).

Loop-mediated isothermal amplification (LAMP): A method for amplifying DNA. The method is a single-step amplification reaction utilizing a DNA polymerase with strand displacement activity (e.g., Notomi et al., Nucl. Acids. Res. 28:E63, 2000; Nagamine et al., Mol. Cell. Probes 16:223-229, 2002; Mori et al., J. Biochem. Biophys. Methods 59:145-157, 2004). At least four primers, which are specific for eight regions within a target nucleic acid sequence, are typically used for LAMP. The primers include a forward outer primer (F3), a backward outer primer (B3), a forward inner primer (FIP), and a backward inner primer (BIP). A forward loop primer (Loop F), and a backward loop primer (Loop B) can also be included in some embodiments. The amplification reaction produces a stem-loop DNA with inverted repeats of the target nucleic acid sequence. Reverse transcriptase can be added to the reaction for amplification of RNA target sequences. This variation is referred to as RT-LAMP.

Primer: Primers are short nucleic acids, generally DNA oligonucleotides 10 nucleotides or more in length (such as 12, 15, 18, 20, 25, 30, 40, 50, or more nucleotides in length). In some examples, primers are 10 to 60 nucleotides long (for example, 15-50, 20-40, 15-35, or 25-50 nucleotides long). Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by PCR, LAMP, RT-LAMP, or other nucleic acid amplification methods known in the art.

Probe: A probe typically comprises an isolated nucleic acid (for example, at least 10, 15, 18, 20, 25, 30, 40, or more nucleotides in length) with an attached detectable label or reporter molecule. In some examples, probes are 15-40 nucleotides long (for example, 15-30, 18-40, or 20-30 nucleotides long). Typical labels include radioactive isotopes, ligands, chemiluminescent agents, fluorophores, and enzymes. Methods for labeling oligonucleotides and guidance in the choice of labels appropriate for various purposes are discussed, e.g., in Sambrook et al. (2001) and Ausubel et al. (1987).

Recombinant nucleic acid: A nucleic acid molecule that is not naturally occurring or has a sequence that is made by an artificial combination of two otherwise separated segments of nucleotide sequence. This artificial combination is accomplished by chemical synthesis or by the artificial manipulation of isolated segments of nucleic acids, e.g., by genetic engineering techniques such as those described in Sambrook and Russell, in Molecular Cloning: A Laboratory Manual, 3^{rd} Ed., Cold Spring Harbor Laboratory Press (2001). The term “recombinant” includes nucleic acids that have been altered solely by addition, substitution, or deletion of a portion of a natural nucleic acid molecule. A recombinant nucleic acid also includes a heterologous nucleic acid that is inserted in a vector. A “heterologous nucleic acid” refers to a nucleic acid that originates from a different genetic source or species, for example a viral nucleic acid inserted in a bacterial plasmid (referred to herein in some examples as a recombinant vector).

Sample (or biological sample): A biological specimen containing DNA (for example, genomic DNA or cDNA), RNA (including mRNA), protein, or combinations thereof. Examples include, but are not limited to isolated nucleic acids, cells, cell lysates, chromosomal preparations, peripheral blood, urine, saliva, tissue biopsy (such as a tumor biopsy or lymph node biopsy), surgical specimen, bone marrow, amniocentesis samples, and autopsy material. In one example, a sample includes viral nucleic acids, for example, HIV RNA or DNA reverse transcribed from HIV RNA. In particular examples, samples are used directly (e.g., fresh or frozen), or can be manipulated prior to use, for example, by fixation (e.g., using formalin) and/or embedding in wax (such as FFPE tissue samples).

Subject: Any multi-cellular vertebrate organism, such as human and non-human mammals (including non-human primates). In one example, a subject is known to be or is suspected of being infected with HIV.

Transduced and Transformed: A virus or vector “transduces” a cell when it transfers nucleic acid into the cell. A cell is “transformed” by a nucleic acid transduced into the cell when the DNA becomes replicated by the cell, either by incorporation of the nucleic acid into the cellular genome, or by episomal replication. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Vector: A nucleic acid molecule that can be introduced into a host cell, thereby producing a transformed or transduced host cell. Recombinant DNA vectors are vectors including recombinant DNA. A vector can include nucleic acid sequences that permit it to replicate in a host cell, such as an origin of replication. A vector can also include one or more selectable marker genes, a cloning site for introduction of heterologous nucleic acids, and/or other genetic elements known in the art. Vectors include plasmid vectors, including plasmids for expression in gram negative and gram positive bacterial cells. Exemplary vectors include those for use in E. coli. Vectors also include viral vectors, such as, but not limited to, retrovirus, orthopox, avipox, fowlpox, capripox, suipox, adenovirus, herpes virus, alpha virus, baculovirus, Sindbis virus, vaccinia virus, and poliovirus vectors. Vectors also include yeast cell vectors. In some examples, a heterologous nucleic acid (such as an HIV-2 nucleic acid) is introduced into a vector to produce a recombinant vector, thereby allowing the viral nucleic acid to be renewably produced.

### II. Methods of Detecting HIV-2 Nucleic Acids

Disclosed herein are methods of detecting HIV-2 nucleic acids in a sample (such as from a sample from a subject infected with or suspected to be infected with HIV-2). In some examples, the methods include LAMP or RT-LAMP, while in other examples, the methods include hybridization of a probe to an HIV-2 nucleic acid, including, but not limited to real-time PCR. In particular examples, the methods include detecting and/or discriminating HIV-2 (for example from HIV-1) or detecting and/or discriminating different HIV-2 groups (such as HIV-2 Group A and/or HIV-2 Group B). Primers and probes specific for HIV-2 and/or HIV-2 Group A or Group B are provided herein.

The methods described herein may be used for any purpose for which detection of HIV nucleic acids, such as HIV-2 nucleic acids, is desirable, including diagnostic and prognostic applications, such as in laboratory and clinical settings. Appropriate samples include any conventional biological samples, including clinical samples obtained from a human or veterinary subject. Suitable samples include all biological samples useful for detection of infection in subjects, including, but not limited to, cells (such as buccal cells or peripheral blood mononuclear cells), tissues, autopsy samples, bone marrow aspirates, bodily fluids (for example, blood, serum, plasma, urine, cerebrospinal fluid, middle ear fluids, bronchoalveolar lavage, tracheal aspirates, sputum, nasopharyngeal aspirates, oropharyngeal aspirates, or saliva), oral swabs, eye swabs, cervical swabs, vaginal swabs, rectal swabs, stool, and stool suspensions. The sample can be used directly or can be processed, such as by adding solvents, preservatives, buffers, or other compounds or substances. In some examples, nucleic acids are isolated from the sample. In other examples, isolation of nucleic acids from the sample is not necessary prior to use in the methods disclosed herein and the sample (such as a blood sample) is used directly. In some examples, the sample is pre-treated with a lysis buffer, but nucleic acids are not isolated prior to use in the disclosed methods.

Samples also include isolated nucleic acids, such as DNA or RNA isolated from a biological specimen from a subject, an HIV isolate, or other source of nucleic acids. Methods for extracting nucleic acids such as RNA and/or DNA from a sample are known to one of skill in the art; such methods will depend upon, for example, the type of sample in which the nucleic acid is found. Nucleic acids can be extracted using standard methods. For instance, rapid nucleic acid preparation can be performed using a commercially available kit (such as kits and/or instruments from Qiagen (such as DNEasy® or RNEasy® kits), Roche Applied Science (such as MagNA Pure kits and instruments), Thermo Scientific (KingFisher mL), bioMérieux (Nuclisens® NASBA Diagnostics), or Epicentre (Masterpure™ kits)). In other examples, the nucleic acids may be extracted using guanidinium isothiocyanate, such as single-step isolation by acid guanidinium isothiocyanate-phenol-chloroform extraction (Chomczynski et al. Anal. Biochem. 162:156-159, 1987).

The disclosed methods are highly sensitive and/or specific for detection of HIV-2 nucleic acids. In some examples, the disclosed methods can detect presence of at least 10 copies of HIV-2 nucleic acids (for example at least 10², 10³, 10⁴, 10⁵, 10⁶, or more copies of HIV-2 nucleic acids) in a sample or reaction volume (such as copies/mL). In some examples, the disclosed methods can predict with a sensitivity of at least 90% and a specificity of at least 90% for presence of an HIV-2 nucleic acid (such as an HIV-2 Group A nucleic acid or an HIV-2 Group B nucleic acid), such as a sensitivity of at least 91%, 92%, 93%, 94%, 95%, 96%, 97%, 98%, 99%, or even 100% and a specificity of at least of at least 91%, 92%, 93%, 94%, 95%, 96%, 97%, 98%, 99%, or even 100%.

A. Loop-Mediated Isothermal Amplification

In some embodiments, the methods for detecting HIV-2 in a sample utilize LAMP or RT-LAMP methods of amplification and detection. LAMP, which was first described by Notomi et al. (Nucl. Acids Res. 28:e63, 2000), is a one-step isothermal amplification method that can produce amplified nucleic acids in a short period of time using a DNA polymerase with strand displacement activity. LAMP can be adapted for amplification of RNA targets with the addition of reverse transcriptase (RT) to the reaction without an additional heat step (referred to as RT-LAMP). The isothermal nature of LAMP and RT-LAMP allows for assay flexibility because it can be used with simple and inexpensive heating devices, which can facilitate HIV detection in settings other than centralized clinical laboratories, including at the point-of-care (POC). POC testing is particularly important for HIV diagnosis, as it has the potential to reduce loss to follow-up and to increase the number of individuals that become aware of their HIV status (for example, at the time of their visit). In addition, LAMP and RT-LAMP offer versatility in terms of specimen type and is believed to increase the probability of detecting an amplifiable target in whole blood specimens or dried blood spots.

LAMP or RT-LAMP can also be multiplexed through the addition of multiple LAMP primer sets with different specificities. This capability is advantageous, for example, because it allows for incorporation of internal control(s), amplification of two or more regions within the same target, or detection of two or more targets or pathogens in a single reaction. In some examples, the disclosed methods include a multiplex LAMP or RT-LAMP assay for detection and/or discrimination of HIV-2 Group A and HIV-2 Group B in a single reaction. In other examples, the disclosed methods include a multiplex LAMP or RT-LAMP assay for detection and/or discrimination of HIV-1 and HIV-2 in a single reaction.

In some embodiments, the methods include contacting a sample (such as a sample including or suspected to include HIV-2 nucleic acids) with at least one set of LAMP primers specific for an HIV-2 integrase nucleic acid under conditions sufficient for amplification of the HW-2 nucleic acid, producing an amplification product. In some examples, the LAMP primers amplify an HIV-2 nucleic acid having at least 80% sequence identity (such as at least 85%, 90%, 95%, 98%, or more sequence identity) to nucleotides 5208-5418 of the Mac239 reference sequence (e.g. GenBank Accession No. M33262, incorporated herein by reference), or a portion thereof. In some examples, the methods further include reverse transcription of HIV-2 RNA in the sample, for example by contacting the sample with a reverse transcriptase. The amplification product is detected by any suitable method, such as detection of turbidity, fluorescence, or by gel electrophoresis.

LAMP primers generally include oligonucleotides between 15 and 60 nucleotides in length. In some embodiments, the set of LAMP primers specifically amplifies an HIV-2 Group A nucleic acid. An exemplary set of LAMP primers for amplification of an HIV-2 Group A nucleic acid includes an F3 primer with at least 90% sequence identity to SEQ ID NO: 23, a B3 primer with at least 90% sequence identity to SEQ ID NO: 24, an FIP primer with at least 90% sequence identity to SEQ ID NO: 25, and a BIP primer with at least 90% sequence identity to SEQ ID NO: 26, or the reverse complement of any thereof. In some examples, the set of LAMP primers for amplification of HIV-2 Group A nucleic acids further includes a Loop F primer with at least 90% sequence identity to SEQ ID NO: 27 and a Loop B primer with at least 90% sequence identity to SEQ ID NO: 28, or the reverse complement of either or both. In some examples, the set of LAMP primers for HIV-2 Group A includes primers comprising, consisting essentially of, or consisting of the nucleic acid sequence each of SEQ ID NOs: 23-26 or SEQ ID NOs: 23-28. In additional examples, the set of LAMP primers further includes a quencher primer with at least 90% sequence identity to SEQ ID NO: 35 or the reverse complement thereof (for example, a quencher primer comprising, consisting essentially of, or consisting of the nucleic acid sequence of SEQ ID NO: 35).

In other embodiments, the set of LAMP primers specifically amplifies an HIV-2 Group B nucleic acid. An exemplary set of LAMP primers for amplification of an HIV-2 Group B nucleic acid includes an F3 primer with at least 90% sequence identity to SEQ ID NO: 29, a B3 primer with at least 90% sequence identity to SEQ ID NO: 30, an FIP primer with at least 90% sequence identity to SEQ ID NO: 31, and a BIP primer with at least 90% sequence identity to SEQ ID NO: 32, or the reverse complement of any thereof. In some examples, the set of LAMP primers for amplification of HIV-2 Group B nucleic acids further includes a Loop F primer with at least 90% sequence identity to SEQ ID NO: 33, and a Loop B primer with at least 90% sequence identity to SEQ ID NO: 34, or the reverse complement thereof. In some examples, the set of LAMP primers for HIV-2 Group B includes primers comprising, consisting essentially of, or consisting of the nucleic acid sequence of each of SEQ ID NOs: 29-32 or SEQ ID NOs: 29-34. In some examples, the set of LAMP primers additionally includes a quencher primer with at least 90% sequence identity to SEQ ID NO: 35 or the reverse complement thereof (for example, a quencher primer comprising, consisting essentially of, or consisting of the nucleic acid sequence of SEQ ID NO: 35).

The LAMP and RT-LAMP methods disclosed herein can be used with a single set of LAMP primers (such as a set of LAMP primers for Group A HIV-2 or Group B HIV-2, for example, those described above). In other examples, the methods include multiplex LAMP or RT-LAMP reactions, which include two or more sets of LAMP primers for amplification of different HIV-2 target nucleic acids, target nucleic acids from different HIV-2 Groups (such as Group A and Group B), or target nucleic acids from different viruses or other pathogens (such as HIV-1 and HIV-2). In a particular example, a multiplex LAMP or RT-LAMP reaction includes a set of Group A HIV-2 LAMP primers (such as SEQ ID NOs: 23-26 or 23-28) and a set of Group B HIV-2 LAMP primers (such as SEQ ID NOs: 29-32 or 29-34), and optionally including a quencher primer (such as SEQ ID NO: 35). In other examples, a multiplex LAMP or RT-LAMP reaction includes at least one set of HIV-2 LAMP primers (such as SEQ ID NOs: 23-26 or 23-28 and/or SEQ ID NOs: 29-32 or 29-34, and optionally SEQ ID NO: 35) and at least one set of additional HIV-2 or HIV-1 LAMP primers (such as those described in Curtis et al., PLoS One 7:e31432, 2012 and U.S. Pat. Publ. No. 2012/0088244, both of which are incorporated by reference herein in their entirety).

The sample and LAMP primer set(s) are contacted under conditions sufficient for amplification of an HIV nucleic acid (such as an HIV-2 nucleic acid). The amount of sample used in the reaction can be selected by one of skill in the art based on the type of sample, the reaction volume, and other parameters. In some example, about 1-20 μL (e.g., about 1-10 μL, about 1-5 μL, or about 5-10 μL) of unextracted sample is included in the reaction. In other examples, about 1-20 μL (about 1-10 μL, about 10-20 μL, about 5-10 μL, or about 1-5 μL) of extracted nucleic acids is included in the reaction.

The sample is contacted with the set of LAMP primers at a concentration sufficient to support amplification of an HIV nucleic acid. In some examples, the amount of each primer is about 0.1 μM to about 5 μM (such as about 0.2 μM to about 2 μM, or about 0.5 μM to about 2 μM). Each primer can be included at a different concentration, and appropriate concentrations for each primer can be selected by one of skill in the art using routine methods. Exemplary primer concentrations are provided in Example 1, below.

In some examples, the LAMP or RT-LAMP reaction is carried out in a mixture including a suitable buffer (such as a phosphate buffer or Tris buffer). The buffer may also include additional components, such as salts (such as KCl or NaCl, magnesium and/or manganese salts (e.g., MgCl₂, MgSO₄, MnCl₂, or MnSO₄), ammonium (e.g., (NH₄)₂SO₄)), detergents (e.g., TRITON®-X100), or other additives (such as betaine or dimethylsulfoxide). One of skill in the art can select an appropriate buffer and any additives using routine methods. In one non-limiting example, the buffer includes 20 mM Tris-HCl, 10 mM (NH₄)₂SO₄, 10 mM KCl, 10 mM MgSO₄, 0.1% TRITON®-X100, and 0.8 M betaine. The reaction mixture also includes nucleotides or nucleotide analogs. In some examples, an equimolar mixture of dATP, dCTP, dGTP, and dTTP (referred to as dNTPs) is included, for example about 0.5-2 mM dNTPs.

A DNA polymerase with strand displacement activity is also included in the reaction mixture. Exemplary DNA polymerases with strand displacement activity include Bst DNA polymerase, Bst 2.0 DNA polymerase, Bst 2.0 WarmStart™ DNA polymerase (New England Biolabs, Ipswich, Mass.), Phi29 DNA polymerase, Bsu DNA polymerase, OmniAmp™ DNA polymerase (Lucigen, Middleton, Mich.), Taq DNA polymerase, VentR® and Deep VentR® DNA polymerases (New England Biolabs), 9° Nm™ DNA polymerase (New England Biolabs), Klenow fragment of DNA polymerase I, PhiPRD1 DNA polymerase, phage M2 DNA polymerase, T4 DNA polymerase, and T5 DNA polymerase. In some examples, about 1 to 20 U (such as about 1 to 15 U, about 2 to 12 U, about 10 to 20 U, about 2 to 10 U, or about 5 to 10 U) of DNA polymerase is included in the reaction. In some examples, the polymerase has strand displacement activity and lacks 5′-3′ exonuclease activity. In one non-limiting example, the DNA polymerase is Bst DNA polymerase.

In some embodiments, the target HIV-2 nucleic acid is RNA, and a reverse transcriptase is additionally included in the LAMP assay (called an RT-LAMP assay). Exemplary reverse transcriptases include MMLV reverse transcriptase, AMV reverse transcriptase, and ThermoScript™ reverse transcriptase (Life Technologies, Grand Island, N.Y.), Thermo-X™ reverse transcriptase (Life Technologies, Grand Island, N.Y.). In some examples, about 0.1 to 50 U (such as about 0.2 to 40 U, about 0.5 to 20 U, about 1 to 10 U, or about 2 to 5 U) of RT is included in the reaction.

The reaction mixture, including sample, LAMP primers, buffers, nucleotides, DNA polymerase, optionally reverse transcriptase, and any other components, is incubated for a period of time and at a temperature sufficient for production of an amplification product. In some examples, the reaction conditions include incubating the reaction mixture at about 37° C. to about 80° C. (such as about 40° C. to about 70° C. or about 50° C. to about 65° C.), for example about 40° C., about 45° C., about 50° C., about 55° C., about 60° C., about 65° C., about 70° C., about 75° C., or about 80° C. The reaction mixture is incubated for at least about 5 minutes (such as about 10, about 15, about 20, about 30, about 40, about 50, about 60, about 70, about 80 about 90, about 100, about 110, about 120 minutes or more), for example about 10-120 minutes, about 15-90 minutes, about 20-70 minutes, or about 30-60 minutes. In particular examples, the reaction mixture is incubated for about 20-70 minutes at about 50° C. to 65° C.

Following incubation of the reaction mixture, the amplification product is detected by any suitable method. The detection method may be quantitative, semi-quantitative, or qualitative. In some examples, accumulation of an amplification product is detected by measuring the turbidity of the reaction mixture (for example, visually or with a turbidometer). In other examples, amplification product is detected using gel electrophoresis, for example by detecting presence or amount of amplification product with agarose gel electrophoresis. In some examples, amplification product is detected using a colorimetric assay, such as with an intercalating dye (for example, propidium iodide, SYBR green or Picogreen) or a chromogenic reagent (see, e.g., Goto et al., BioTechniques 46:167-172, 2009). In other examples, the disclosed methods include calcein in the reaction (such as about 5 μM to about 50 μM, for example, about 10-50 μM or about 6-25 μM), which provides for fluorescent detection of the amplification product (see, e.g., Tomita et al., Nature Protocols 3:877-892, 2008). Calcein is a fluorescence indicator dye that is quenched by manganese ions and has increased fluorescence when bound to magnesium ions. The LAMP assay produces large amounts of pyrophosphate, which strongly binds to metal ions (particularly manganese and magnesium) and forms an insoluble precipitate. Thus, in some examples, LAMP assays including calcein include both manganese (e.g., MnCl₂ or MnSO₄) and magnesium (MgCl₂ or MgSO₄). As the amplification reaction proceeds, pyrophosphate is produced and competes with calcein for binding to Mn²⁺. This reduces the quenching of the calcein, and also allows Mg²⁺ to bind to the calcein, further increasing its fluorescence.

In other examples, amplification products are detected using a detectable label incorporated in one or more of the LAMP primers (discussed below). The detectable label may be optically detectable, for example, by eye or using a spectrophotometer or fluorimeter. In some examples, the detectable label is a fluorophore, such as those described above. In some examples, the label is detected in real-time, for example using a fluorescence scanner (such as ESEQuant, Qiagen). One of skill in the art can select one or more detectable labels for use in the methods disclosed herein.

In particular embodiments, one of the LAMP primers includes a detectable label, such as a fluorophore. In a specific example, the Loop B primer (for example, SEQ ID NOs: 28 or 34) includes a fluorophore, for example attached to the 5′ end or the 3′ end of the primer. Any fluorophore can be used; in one non-limiting example, the fluorophore is HEX. In embodiments including a quencher primer, the quencher includes an acceptor fluorophore (a quencher). The quencher primer is complementary to the labeled primer and reduces or even substantially eliminates detectable fluorescence from the labeled primer if the labeled primer is not incorporated in the LAMP amplification product, thus reducing background or non-specific fluorescence in the reaction. In some examples, the quencher primer includes a BLACK HOLE quencher, for example, attached to the 5′ end or the 3′ end of the primer. Exemplary quenchers include BHQ1, BHQ2, or BHQ3.

B. Probe Hybridization Methods

In some embodiments, the methods include contacting a sample (such as a sample including or suspected to include HIV-2 nucleic acids) with at least one probe comprising a nucleic acid molecule between 10 and 40 nucleotides in length (such as 15-40, 20-40, or 15-30 nucleotides long) and detecting hybridization between the one or more probes and an HIV-2 nucleic acid an HIV-2 nucleic acid in the sample. In some examples, the probe is capable of hybridizing under very high stringency conditions to an HIV-2 LTR nucleic acid (such as SEQ ID NOs: 1-11), an HIV-2 pol nucleic acid (such as SEQ ID NOs: 12-22), or a nucleic acid sequence at least 90% identical (for example 91%, 92%, 93%, 94%, 95%, 96%, 97%, 98%, 99%, or even 100% identical) to one of SEQ ID NOs: 1-22. In some examples, the sample is contacted with one or more nucleic acid probes between 20 and 40 nucleotides in length comprising or consisting of a nucleic acid sequence at least 90% identical (for example 91%, 92%, 93%, 94%, 95%, 96%, 97%, 98%, 99%, or even 100% identical) to any one of SEQ ID NOs: 55, 56, 60-62, 68, 69, 77, 78, or the reverse complement thereof.

In additional embodiments, the probe is capable of hybridizing under very high stringency conditions to an HIV-2 Env nucleic acid or an HIV-2 LTR-gag nucleic acid. In some examples, the sample is contacted with one or more nucleic acid probes between 20 and 40 nucleotides in length comprising or consisting of a nucleic acid sequence at least 90% identical (for example 91%, 92%, 93%, 94%, 95%, 96%, 97%, 98%, 99%, or even 100% identical) to any one of SEQ ID NOs: 86, 90, or the reverse complement thereof.

In some embodiments of the methods described herein, the sample is further contacted with a control probe. In some examples, the probe is capable of hybridizing under very high stringency conditions to a human nucleic acid. In some examples, the sample is contacted with a nucleic probe capable of hybridizing to a human RNase P nucleic acid, for example a probe comprising or consisting of a nucleic acid sequence at least 90% identical (for example 91%, 92%, 93%, 94%, 95%, 96%, 97%, 98%, 99%, or even 100% identical) to any one of SEQ ID NO: 94, or the reverse complement thereof.

In some examples, the probes are at least 10, 15, 20, 25, 30, 35, or 40 nucleotides in length. In other examples, the probes may be no more than 10, 15, 20, 25, 30, 35, or 40 nucleotides in length. In further examples, the probes are 15, 16, 17, 18, 19, 20, 21, 22, 23, 24, 25, 26, 27, 28, 29, 30, 31, 32, 33, 34, 35, 36, 37, 38, 39, or 40 nucleotides in length. In some embodiments, the probe is detectably labeled, either with an isotopic or non-isotopic label; in alternative embodiments, the target nucleic acid is labeled. Non-isotopic labels can, for instance, comprise a fluorescent or luminescent molecule, or an enzyme, co-factor, enzyme substrate, or hapten. The probe is incubated with a sample including single-stranded or double-stranded RNA, DNA, or a mixture of both, and hybridization is determined. In some examples, the hybridization results in a detectable change in signal such as in increase or decrease in signal, for example from the labeled probe. Thus, detecting hybridization comprises detecting a change in signal from the labeled probe during or after hybridization relative to signal from the label before hybridization.

In some examples, the probe is labeled with one or more fluorophores. Examples of suitable fluorophore labels are provided above. In some examples, the fluorophore is a donor fluorophore. In particular, non-limiting examples, the probes disclosed herein are labeled with CalRed610, although one of skill in the art can select other fluorophore labels for use in the disclosed methods (including, but not limited to FAM, HEX, or CalRed590). In other examples, the fluorophore is an accepter fluorophore, such as a fluorescence quencher. In some examples, the probe includes both a donor fluorophore and an acceptor or quencher fluorophore, for example a donor fluorophore such as CalRed610 and an acceptor fluorophore such as a BLACK HOLE® quencher (such as BHQ1, BHQ2, or BHQ3). Appropriate donor/acceptor fluorophore pairs can be selected using routine methods. In one example, the donor fluorophore emission wavelength is one that can significantly excite the acceptor fluorophore, thereby generating a detectable emission from the acceptor fluorophore. In some examples, the probe is modified at the 3′-end to prevent extension of the probe by a polymerase.

In some embodiments, HIV-2 nucleic acids present in a sample are amplified prior to or concurrently with using a probe for detection. For instance, it can be advantageous to amplify a portion of one of more of the disclosed nucleic acids, and then detect the presence of the amplified nucleic acid, for example, to increase the number of nucleic acids that can be detected, thereby increasing the signal obtained. Specific nucleic acid primers can be used to amplify a region that is at least about 50, at least about 60, at least about 70, at least about 80 at least about 90, at least about 100, at least about 200, at least about 250, at least about 300, at least about 400, at least about 500, at least about 1000, at least about 2000, or more base pairs in length to produce amplified nucleic acids. In other examples, specific nucleic acid primers can be used to amplify a region that is about 50-3000 base pairs in length (for example, about 70-2000 base pairs, about 100-1000 base pairs, about 50-300 base pairs, about 50-100 base pairs, about 300-500 base pairs, or about 1000-3000 base pairs in length).

Detecting the amplified product typically includes the use of labeled probes that are sufficiently complementary to and hybridize to the amplified nucleic acid sequence. Thus, the presence, amount, and/or identity of the amplified product can be detected by hybridizing a labeled probe, such as a fluorescently labeled probe, complementary to the amplified product. In one embodiment, the detection of an HIV-2 nucleic acid sequence of interest, such as an HIV-2 LTR or pol nucleic acid includes the combined use of PCR amplification and a labeled probe such that the product is measured using real-time PCR (such as TaqMan® real-time PCR). In another embodiment, the detection of an amplified target nucleic acid sequence of interest includes the transfer of the amplified target nucleic acid to a solid support, such as a membrane, for example a Northern blot or a Southern blot, and contacting the membrane with a probe, for example a labeled probe, that is complementary to at least a portion of the amplified target nucleic acid sequence. In still further embodiments, the detection of an amplified target nucleic acid of interest includes the hybridization of a labeled amplified target nucleic acid to probes disclosed herein that are arrayed in a predetermined array with an addressable location and that are complementary to the amplified target nucleic acid.

Any nucleic acid amplification method can be used in the methods disclosed herein to detect the presence of one or more HIV-2 nucleic acids in a sample. In one specific, non-limiting example, polymerase chain reaction (PCR) is used to amplify the pathogen-specific nucleic acid sequences. In other specific, non-limiting examples, real-time PCR, reverse transcriptase-polymerase chain reaction (RT-PCR), real-time reverse transcriptase-polymerase chain reaction (rt RT-PCR), ligase chain reaction, or transcription-mediated amplification (TMA) is used to amplify the nucleic acids. In a specific example, one or more HIV-2 nucleic acids are amplified by real-time PCR, for example real-time TaqMan® PCR. In some examples, the HIV-2 nucleic acids are HIV-2 DNA, which has been reversed transcribed from RNA using reverse transcriptase. Techniques for reverse transcription and nucleic acid amplification are well-known to those of skill in the art.

Typically, at least two primers are utilized in the amplification reaction. In some examples, amplification of an HIV-2 nucleic acid involves contacting the nucleic acid with one or more primers (such as two or more primers) that are capable of hybridizing to and directing the amplification of at least a portion of an HIV-2 LTR nucleic acid, such as a primer capable of hybridizing under very high stringency conditions to an HIV-2 LTR nucleic acid sequence (such as an LTR sequence set forth as any one of SEQ NOs: 1-11), for example a primer that is least 90% identical (such as 91%, 92%, 93%, 94%, 95%, 96%, 97%, 98%, 99%, or 100% identical) to the nucleotide sequence set forth as one of SEQ ID NOs: 53, 54, 57, 58, 59, 63, 64, or the reverse complement thereof. In one example, an HW-2 LTR nucleic acid is amplified utilizing a pair of primers, such as a forward primer at least 90% identical to SEQ ID NO: 53 or 54 and a reverse primer at least 90% identical to SEQ ID NO: 57, such as a forward primer comprising or consisting essentially of SEQ ID NO: 53 or 54 and a reverse primer comprising or consisting essentially of SEQ ID NO: 57. In another example, an HIV-2 LTR nucleic acid is amplified utilizing a pair of primers, such as a forward primer at least 90% identical to SEQ ID NO: 58 or 59 and a reverse primer at least 90% identical to SEQ ID NO: 63 or 64, such as a forward primer comprising or consisting essentially of SEQ ID NO: 58 or 59 and a reverse primer comprising or consisting essentially of SEQ ID NO: 63 or 64.

In other examples, amplification of an HIV-2 nucleic acid involves contacting the nucleic acid with one or more primers (such as two or more primers) that are capable of hybridizing to and directing the amplification of an HIV-2 pol nucleic acid, such as a primer capable of hybridizing under very high stringency conditions to an HIV-2 pol nucleic acid sequence (such as a pol sequence set forth as any one of SEQ ID NOs: 12-22), for example a primer that is least 90% identical (such as 91%, 92%, 93%, 94%, 95%, 96%, 97%, 98%, 99%, or 100% identical) to the nucleotide sequence set forth as one of SEQ ID NOs: 65, 66, 67, 70, 71, 72, 73, 74, 75, 76, 79, 80, 81, 82, 83, or the reverse complement thereof. In one example, an HIV-2 pol nucleic acid (such as at least a portion of a protease-encoding nucleic acid) is amplified utilizing a pair of primers, such as a forward primer at least 90% identical to SEQ ID NO: 65, 66, or 67 and a reverse primer at least 90% identical to SEQ ID NO: 70, 71, or 72, such as a forward primer comprising or consisting essentially of SEQ ID NO: 65, 66, or 67 and a reverse primer comprising or consisting essentially of SEQ ID NO: 70, 71, or 72. In another example, an HW-2 pol nucleic acid (such as at least a portion of an integrase-encoding nucleic acid) is amplified utilizing a pair of primers, such as a forward primer at least 90% identical to SEQ ID NO: 73, 74, 75, or 76 and a reverse primer at least 90% identical to SEQ ID NO: 79, 80, 81, 82, or 83, such as a forward primer comprising or consisting essentially of SEQ ID NO: 73, 74, 75, or 76 and a reverse primer comprising or consisting essentially of SEQ ID NO: 79, 80, 81, 82, or 83.

In further examples, amplification of an HW-2 nucleic acid involves contacting the nucleic acid with one or more primers (such as two or more primers) that are capable of hybridizing to and directing the amplification of an HIV-2 env nucleic acid, such as a primer capable of hybridizing under very high stringency conditions to an HIV-2 env nucleic acid sequence, for example a primer that is least 90% identical (such as 91%, 92%, 93%, 94%, 95%, 96%, 97%, 98%, 99%, or 100% identical) to the nucleotide sequence set forth as one of SEQ ID NOs: 84, 85, 87, or 88, or the reverse complement thereof. In one example, an HW-2 env nucleic acid is amplified utilizing a pair of primers, such as a forward primer at least 90% identical to SEQ ID NO: 84 or 85 and a reverse primer at least 90% identical to SEQ ID NO: 87 or 88, such as a forward primer comprising or consisting essentially of SEQ ID NO: 84 or 85 and a reverse primer comprising or consisting essentially of SEQ ID NO: 87 or 88.

In still further examples, amplification of an HIV-2 nucleic acid involves contacting the nucleic acid with one or more primers (such as two or more primers) that are capable of hybridizing to and directing the amplification of an HIV-2 LTR-gag nucleic acid, such as a primer capable of hybridizing under very high stringency conditions to an HIV-2 LTR-gag nucleic acid sequence, for example a primer that is least 90% identical (such as 91%, 92%, 93%, 94%, 95%, 96%, 97%, 98%, 99%, or 100% identical) to the nucleotide sequence set forth as one of SEQ ID NOs: 89, 91, or 92, or the reverse complement thereof. In one example, an HIV-2 LTR-gag nucleic acid is amplified utilizing a pair of primers, such as a forward primer at least 90% identical to SEQ ID NO: 89 and a reverse primer at least 90% identical to SEQ ID NO: 91 or 92, such as a forward primer comprising or consisting essentially of SEQ ID NO: 89 and a reverse primer comprising or consisting essentially of SEQ ID NO: 91 or 92.

In some embodiments, the methods disclosed herein that include detecting presence of HIV-2 nucleic acid in a sample utilize real-time PCR. Real-time PCR monitors the fluorescence emitted during the reaction as an indicator of amplicon production during each PCR cycle, as opposed to endpoint detection. The real-time progress of the reaction can be viewed in some systems. Typically, real-time PCR uses the detection of a fluorescent reporter. In some examples, the fluorescent reporter signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed.

In one embodiment, the fluorescently-labeled probes (such as probes disclosed herein) rely upon fluorescence resonance energy transfer (FRET), or in a change in the fluorescence emission wavelength of a sample, as a method to detect hybridization of a DNA probe to the amplified target nucleic acid in real-time. For example, FRET that occurs between fluorogenic labels on different probes (for example, using HybProbes) or between a donor fluorophore and an acceptor or quencher fluorophore on the same probe (for example, using a molecular beacon or a TaqMan® probe) can identify a probe that specifically hybridizes to the nucleic acid of interest and in this way, can detect the presence and/or amount of the nucleic acid in a sample.

In some embodiments, the fluorescently-labeled probes used to identify amplification products have spectrally distinct emission wavelengths, thus allowing them to be distinguished within the same reaction tube, for example in multiplex PCR, such as a multiplex real-time PCR. In some embodiments, the probes and primers disclosed herein are used in multiplex real-time PCR. For example, multiplex PCR permits the simultaneous detection and/or discrimination of Group A and Group B HIV-2 nucleic acids in a sample. In other examples, multiplex PCR includes detection and/or discrimination of HIV-1 and HIV-2 nucleic acids in a sample. Exemplary HIV-1 oligonucleotides suitable for multiplex PCR (such as multiplex PCR with the HW-2 oligonucleotides disclosed herein) include those described in Luo et al. (J. Clin. Microbiol. 43:1851-1857, 2005). Multiplex PCR reactions may also include one or more primers and/or probes for detection of a control nucleic acid. In one example, a control nucleic acid includes RNase P. Exemplary primers and probes for amplification and detection of RNase P include SEQ ID NOs: 93-95.

### III. Primers, Probes, and Kits

Probes and primers (such as isolated nucleic acid primers and/or probes) suitable for use in the disclosed methods are described herein. In some examples, the probes and primers are suitable for detection of HIV-2 nucleic acids using LAMP. In other examples, the probes and primers are suitable for detection of HIV-2 utilizing PCR-based methods, including real-time PCR. In still further examples, primers for amplifying one or more HIV-2 nucleic acids are disclosed.

In some embodiments, the disclosed primers and/or probes are between 10 and 60 nucleotides in length, such as 10, 11, 12, 13, 14, 15, 16, 17, 18, 19, 20, 21, 22, 23, 24, 25, 26, 27, 28 29, 30, 31, 32, 32, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length and are capable of hybridizing to, and in some examples, amplifying the disclosed nucleic acid molecules. In some examples, the primers and/or probes are at least 10, 15, 20, 25, 30, 35, 40, 45, 50, 55, or 60 nucleotides in length. In other examples, the primers and/or probes may be no more than 10, 15, 20, 25, 30, 35, 40, 45, 50, 55, or 60 nucleotides in length.

In some examples, the disclosed primers include LAMP primers for amplification of HIV-2 Group A nucleic acids, including primers with at least 90% sequence identity to CCTTACAATCCACAAAGCCAA (F3, SEQ ID NO: 23), ATTGTATTTCTTGTTCTGTGGTG (B3, SEQ ID NO: 24), CTGTATTTGCCTGYTCTCTAATTCTTTTTTAGTAGAAGCAATGAATCACC (FIP, SEQ ID NO: 25), AGTACTAATGGCAGTTCATTGCATGTTTTGTCTTTCTGCTGGGGTCAT (BIP, SEQ ID NO: 26), ACTTATCTGATTTTTTAG (Loop F, SEQ ID NO: 27), AATTTTAAAAGAAGGGGAGGA (Loop B, SEQ ID NO: 28), and/or CCTTCTTTTAAAATT (quencher, SEQ ID NO: 35). In other examples, the disclosed primers include LAMP primers for amplification of HIV-2 Group B nucleic acids, including primers with at least 90% sequence identity to CCCTATAACCCACAAAGTCAG (F3, SEQ ID NO: 29), ATTGTATTTCTTGTTCTGTGGTT (B3, SEQ ID NO: 30), TTGATACTGCCTGRTCTCTGATTCTTTTTTAGTAGAAGCAATGAACCATC (FIP, SEQ ID NO: 31), TGTACTAATGGCAGCTCACTGCATGTTTTGTCTTTCTGCAGGGGTCAT (BIP, SEQ ID NO: 32), GTCTATTTGATTTTTTAG (Loop F, SEQ ID NO: 33), AATTTTAAAAGAAGGGGAGGA (Loop B, SEQ ID NO: 34), and CCTTCTTTTAAAATT (quencher, SEQ ID NO: 35). In some examples, at least one of the primers includes a detectable label, such as a fluorophore. In particular examples, the Loop B primer (e.g., SEQ ID NO: 28 or 34) includes a fluorophore at the 5′ or 3′ end, which is HEX in one non-limiting example. In other examples, the quencher (e.g., SEQ ID NO: 35) includes a fluorescence quencher at the 5′ or 3′ end, such as a dark quencher, which is BHQ1 in one non-limiting example.

In some examples, the disclosed probes (such as isolated nucleic acid probes) include probes capable of hybridizing to an HIV-2 nucleic acid, such as an HIV-2 LTR nucleic acid, an HW-2 protease encoding nucleic acid, or an HIV-2 integrase encoding nucleic acid. In one example, the probe is capable of hybridizing to an HIV-2 LTR nucleic acid and is at least 90% identical to the nucleic acid sequence TCCAGCACTAGCAGGTAGAGCC (SEQ ID NO: 55), at least 90% identical to the nucleic acid sequence CTCCAGCACTARCAGGTAGAGCCT (SEQ ID NO: 56), at least 90% identical to the nucleic acid sequence ACACCGARTGACCAGGCGGC (SEQ ID NO: 60), at least 90% identical to the nucleic acid sequence CCGCCTGGTCATYCGGTGTTCA (SEQ ID NO: 61), or at least 90% identical to the nucleic acid sequence CCGCCTGGTCATTCGGTGCTCC (SEQ ID NO: 62). In other examples, the probe is capable of hybridizing to an HW-2 protease-encoding nucleic acid and is at least 90% identical to the nucleic acid sequence TGCTGCACCTCAATTCTCTCTTTGG (SEQ ID NO: 68) or TGCTGTGCCTCAATTCTCTCTTTGG (SEQ ID NO: 69). In still other examples, the probe is capable of hybridizing to an HW-2 integrase-encoding nucleic acid and is at least 90% identical to the nucleic acid sequence TCATATCCCCTATTCCTCCCCTTC (SEQ ID NO: 77) or at least 90% identical to the nucleic acid sequence AGGGGAGGAATAGGGGATATGACYCC (SEQ ID NO: 78). In further examples, the probe is capable of hybridizing to an HIV-2 env nucleic acid and is at least 90% identical to the nucleic acids sequence AGTGCAGCARCAGCAACAGCTG (SEQ ID NO: 86). In other examples, the probe is capable of hybridizing to an HIV-2 LTR-gag nucleic acid and is at least 90% identical to the nucleic acid sequence AGTGARGGCAGTAAGGGCGGC (SEQ ID NO: 90). In some examples, the probe further includes a detectable label. The label may be attached to the 5′ or 3′ end of the probe or may be internal to the probe (such as a labeled nucleotide incorporated into the probe). In some examples, the probe includes at least one fluorophore (such as a fluorescence donor and a fluorescence acceptor). In a specific non-limiting example, the fluorophore includes CalRed610 and/or BHQ2

In additional examples, the disclosed primers (such as isolated nucleic acid primers) include primers for amplification of one or more HIV-2 nucleic acids. The primers include primers capable of amplifying at least a portion of an HIV-2 LTR nucleic acid, such as primers at least 90% identical to any one of TGGAAGGGATGTTTTACAGTGAG (SEQ ID NO: 36), TGGAAGGGATTTACTATAGTGAGAGA (SEQ ID NO: 37), TGGAAGGGATTTTTTATAGTGAAAGAAGAC (SEQ ID NO: 38), GGATTTTCCTGCCTTGGTTT (SEQ ID NO: 39), TCCCGCTCCTCACGCTG (SEQ ID NO: 40), CAGGAAAATCCCTAGCAGGTTG (SEQ ID NO: 41), TGCTAGGGATTTTCCTGCCTCCGTTTC (SEQ ID NO: 42), CAACCTGCTAGGGATTTTCCTG (SEQ ID NO: 43), CGGAGAGGCTGGCAGATYGAG (SEQ ID NO: 53), GGCAGAGGCTGGCAGATTGAG (SEQ ID NO: 54), GGTGAGAGTCYAGCAGGGAACAC (SEQ ID NO: 57), GTGTGTGTTCCCATCTCTCCTAGTCG (SEQ ID NO: 58), GTGTGTGYTCCCATCTCTCCTAGTCG (SEQ ID NO: 59), GCAGAAAGGGTCCTAACAGACCAGG (SEQ ID NO: 63), and GCRAGAAGGGTCCTAACAGACCAGG (SEQ ID NO: 64).

The primers also include primers capable of amplifying an HIV-2 pol nucleic acid, such as primers at least 90% identical to any one of CAACAGCACCCCCAGTAGAT (SEQ ID NO: 44), GGAAAGAAGCCTCGCAACTT (SEQ ID NO: 45), AGCCAAGCAATGCAGGGCTCCTAG (SEQ ID NO: 46), ATCTTGGCTTTCCTRCTTGG (SEQ ID NO: 47), GGCACTACAATCCAATTCTT (SEQ ID NO: 48), TGCAAGTCCACCAAGCCCAT (SEQ ID NO: 49), ATAGTCRRTGATGATCTTYGCRTTCCT (SEQ ID NO: 50), CCAAGTGGGAACCACTATCC (SEQ ID NO: 51), and GTTGCAATTCTCCTGTTCTATGCTTCAGAT (SEQ ID NO: 52).

In other examples, the primers are capable of amplifying at least a portion of an HIV-2 protease-encoding nucleic acid such as primers at least 90% identical to CACCACACAGAGAGGCGACAGAGGA (SEQ ID NO: 65), CACCATGCAGGGARACGACAGAGGA (SEQ ID NO: 66), GACCCTACAAGGAGGTGACRGAGGA (SEQ ID NO: 67), TGACCCTCRATGTRTGCTGTGACTACTGGTC (SEQ ID NO: 70), TGACCCTCRATACATGCTTTGACTACTGGTC (SEQ ID NO: 71), and TGACCCTCGATATATGCTTGGACTACTGGTC (SEQ ID NO: 72).

In still further examples, the primers are capable of amplifying at least a portion of an HIV-2 integrase-encoding nucleic acid such as primers at least 90% identical to TAATGGCAGYTCAYTGCATGAATTTTAAAAG (SEQ ID NO: 73), TRATGGCAACWCACTGCATGAATTTTAAAAG (SEQ ID NO: 74), AGTAYTAATGGCAGTTCAYTGCATGAATTT (SEQ ID NO: 75), TGTACTAATGGCAGCTCAYTGCATGAATTT (SEQ ID NO: 76), GGAGGAATTGTATYTCTTGTTCTGTGGTRAT (SEQ ID NO: 79), GGARGAATTGTATTTCTTGTTCTGTRGTTAT (SEQ ID NO: 80), GGAAGAACTGTATTTCTTGCTCTGTGGTTAT (SEQ ID NO: 81), GGAGGAATTGTATTTCTTGTTCTGTGGTIATCAT (SEQ ID NO: 82), and GGAAGAATTGTATTTCTTGYTCTGTGGTTATCAT (SEQ ID NO: 83).

In other examples, the primers are capable of amplifying at least a portion of an HIV-2 env nucleic acid, such as primers at least 90% identical to CTCGGACTTTAYTGGCCGGGA (SEQ ID NO: 84), CCCGGACTTTAYTGGCTGGGA (SEQ ID NO: 85), CCCCAGACGGTCAGYCGCAACA (SEQ ID NO: 87), and CCCCAGACGGTCAATCTCAACA (SEQ ID NO: 88). In additional examples, the primers are capable of amplifying at least a portion of an HIV-2 LTR-gag nucleic acid, such as primers at least 90% identical to TTGGCGCCYGAACAGGGAC (SEQ ID NO: 89), GCACTCCGTCGTGGTTTGTTCCT (SEQ ID NO: 91), and GCWCTCCGTCGTGGTTGATTCCT (SEQ ID NO: 92).

Although exemplary probe and primer sequences are provided in herein, the primer and/or probe sequences can be varied slightly by moving the probe or primer a few nucleotides upstream or downstream from the nucleotide positions that they hybridize to on the target nucleic molecule acid, provided that the probe and/or primer is still specific for the target nucleic acid sequence. For example, variations of the probes and primers disclosed as SEQ ID NOs: 23-95 can be made by “sliding” the probes or primers a few nucleotides 5′ or 3′ from their positions, and such variations will still be specific for the respective target nucleic acid sequence.

Also provided by the present disclosure are probes and primers that include variations to the nucleotide sequences shown in any of SEQ ID NOs: 23-95, as long as such variations permit detection of the target nucleic acid molecule. For example, a probe or primer can have at least 90% sequence identity such as at least 91%, 92%, 93%, 94%, 95%, 96%, 97%, 98%, or 99% identity to a nucleic acid including the sequence shown in any of SEQ ID NOs: 23-95. In such examples, the number of nucleotides does not change, but the nucleic acid sequence shown in any of SEQ ID NOs: 23-95 can vary at a few nucleotides, such as changes at 1, 2, 3, 4, or 5 nucleotides.

The present application also provides probes and primers that are slightly longer or shorter than the nucleotide sequences shown in any of SEQ ID NOs: 23-95, as long as such deletions or additions permit amplification and/or detection of the desired target nucleic acid molecule (such as one of SEQ ID NOs: 1-22). For example, a probe or primer can include a few nucleotide deletions or additions at the 5′- or 3′-end of the probe or primers shown in any of SEQ ID NOs: 23-95, such as addition or deletion of 1, 2, 3, or 4 nucleotides from the 5′- or 3′-end, or combinations thereof (such as a deletion from one end and an addition to the other end). In such examples, the number of nucleotides changes.

Also provided are probes and primers that are degenerate at one or more positions (such as 1, 2, 3, 4, 5, or more positions), for example, a probe or primer that includes a mixture of nucleotides (such as 2, 3, or 4 nucleotides) at a specified position in the probe or primer. In some examples, the probes and primers disclosed herein include one or more synthetic bases or alternative bases (such as inosine). In other examples, the probes and primers disclosed herein include one or more modified nucleotides or nucleic acid analogues, such as one or more locked nucleic acids (see, e.g., U.S. Pat. No. 6,794,499) or one or more superbases (Nanogen, Inc., Bothell, Wash.). In other examples, the probes and primers disclosed herein include a minor groove binder conjugated to the 5′ or 3′ end of the oligonucleotide (see, e.g., U.S. Pat. No. 6,486,308).

The nucleic acid primers and probes disclosed herein can be supplied in the form of a kit for use in the detection or amplification of one or more HIV-2 nucleic acids. In such a kit, an appropriate amount of one or more of the nucleic acid probes and/or primers (such as one or more of SEQ ID NOs: 23-95) are provided in one or more containers or in one or more individual wells of a multiwell plate or card. A nucleic acid probe and/or primer may be provided suspended in an aqueous solution or as a freeze-dried or lyophilized powder, for instance. The container(s) in which the nucleic acid(s) are supplied can be any conventional container that is capable of holding the supplied form, for instance, microfuge tubes, multi-well plates, ampoules, or bottles. The kits can include either labeled or unlabeled nucleic acid probes (for example, 1, 2, 3, 4, 5, or more probes) and/or primers (for example, 1, 2, 3, 4, 5, 6, 7, 8, 9, 10, 11, 12, 13, 14, 15, or more primers) for use in amplification and/or detection of HIV-2 nucleic acids. One or more control probes, primers, and or nucleic acids also may be supplied in the kit. An exemplary control is RNase P; however one of skill in the art can select other suitable controls. In some examples, one or more of the probes or primers are detectably labeled.

In some examples, one or more probes and/or one or more primers (such as one or more pairs of primers), may be provided in pre-measured single use amounts in individual, typically disposable, tubes, wells, or equivalent containers. In this example, the sample to be tested for the presence of the target nucleic acids can be added to the individual tube(s) or well(s) and amplification and/or detection can be carried out directly.

In some embodiments, the kits include at least one set of LAMP primers for amplification and/or detection of HIV-2 nucleic acids. In one example, the kit includes a set of primers including SEQ ID NOs: 23-26 and optionally SEQ ID NO: 35. In another example, the kit includes a set of primers including SEQ ID NOs: 23-28, and optionally SEQ ID NO: 35. In a further example, the kit includes a set of primers including SEQ ID NOs: 29-32 and optionally SEQ ID NO: 35 or SEQ ID NOs: 29-34, and optionally SEQ ID NO: 35. In yet another example, the kit includes two sets of LAMP primers, including SEQ ID NOs: 23-26 or 23-28 and SEQ ID NOs: 29-32 or 29-34, the kit optionally also including SEQ ID NO: 35.

In other embodiments, the kit includes at least one probe and a pair of primers (such as a forward primer and a reverse primer) for real-time PCR detection of HIV-2. In some examples, the kit includes at least one probe comprising the sequence of SEQ ID NO: 55 or SEQ ID NO: 56 (for example, SEQ ID NO: 55 or SEQ ID NO: 56 with a detectable label), at least one forward primer comprising the sequence of SEQ ID NO: 53 or SEQ ID NO: 54 and a reverse primer comprising the sequence of SEQ ID NO: 57. In other examples, the kit includes at least one probe comprising the sequence of any one of SEQ ID NOs: 60-62 (for example, SEQ ID NO: 60, 61, or 62 with a detectable label), at least one forward primer comprising the sequence of SEQ ID NO: 58 or SEQ ID NO: 59 and at least one reverse primer comprising the sequence of SEQ ID NO: 63 or SEQ ID NO: 64. In additional examples, the kit includes at least one probe comprising the sequence of SEQ ID NO: 68 or SEQ ID NO: 69 (for example, SEQ ID NO: 68 or 69 with a detectable label), at least one forward primer comprising the sequence of any one of SEQ ID NOs: 65-67 and at least one reverse primer comprising the sequence of any one of SEQ ID NOs: 70-72. In still further examples, the kit includes a probe comprising the nucleic acid sequence of SEQ ID NO: 77 or SEQ ID NO: 78 (for example, SEQ ID NO: 77 or 78 with a detectable label), at least one forward primer comprising the sequence of any one of SEQ ID NO: 73-76, and at least one reverse primer comprising the nucleic acid sequence of any one of SEQ ID NOs: 79-83. In other examples, the kit includes a probe comprising the nucleic acid sequence of SEQ ID NO: 86 (for example, SEQ ID NO: 86 with a detectable label), at least one forward primer comprising the sequence of SEQ ID NO: 84 or SEQ ID NO: 85, and at least one reverse primer comprising the nucleic acid sequence of SEQ ID NO: 87 or SEQ ID NO: 88. In additional examples, the kit includes a probe comprising the nucleic acid sequence of SEQ ID NO: 90 (for example, SEQ ID NO: 90 with a detectable label), at least one forward primer comprising the sequence of SEQ ID NO: 89, and at least one reverse primer comprising the nucleic acid sequence of SEQ ID NO: 91 or SEQ ID NO: 92.

In other embodiments, the kit includes at least two primers (for example, at least one pair of primers) for amplification of HIV-2 nucleic acids. In some examples, the kit includes at least one forward primer selected from SEQ ID NOs: 36-38 and at least one reverse primer selected from SEQ ID NOs: 39-43. In other examples, the kit includes at least one forward primer selected from SEQ ID NOs: 44-46 and at least one reverse primer selected from SEQ ID NOs: 47-52.

The kits disclosed herein may also include one or more control probes and/or primers. In some examples, the kit includes at least one probe that is capable of hybridizing to an RNase P nucleic acid and/or one or more primers capable of amplifying an RNase P nucleic acid. In a particular example, the kit includes a probe comprising a nucleic acid sequence at least 90% identical to TGACCTGAAGGCTCTGCGCG (SEQ ID NO: 94), at least one forward primer at least 90% identical to GTGTTTGCAGATTTGGACCTGCG (SEQ ID NO: 93), and/or at least one reverse primer at least 90% identical to AGGTGAGCGGCTGTCTCCAC (SEQ ID NO: 95).

### IV. HIV-2 Clones and Methods of Use

Disclosed herein are isolated HIV-2 LTR and pol nucleic acids from different HIV-2 clinical isolates, including HIV-2 Group A and HIV-2 Group B isolates. In some embodiments, the disclosed HIV-2 nucleic acids are useful as standards for HIV-2 nucleic acid amplification test development and/or validation. The disclosed HIV-2 nucleic acids may also be used in Quality Control and Quality Assurance programs for clinical use of HIV-2 nucleic acid amplification tests.

In some embodiments, the HIV-2 LTR nucleic acids include or consist of the nucleic acid sequence set forth as any one of SEQ ID NOs: 1-11. In other embodiments, HIV-2 pol nucleic acids include or consist of the nucleic acid sequence set forth as any one of SEQ ID NOs: 12-22. In further embodiments, an isolated HIV-2 nucleic acid molecule disclosed herein has a sequence having at least 90%, 91%, 92%, 93%, 94%, 95%, 96%, 97%, 98%, 99%, or more sequence identity to the nucleic acid sequence set forth in one of SEQ ID NOs: 1-22. In one example, the nucleic acid retains a function of the LTR or the encoded pol protein(s). In some embodiments, the disclosed nucleic acid molecules are incorporated into a vector (such as an autonomously replicating plasmid or virus), or alternatively exist as a separate molecule (such as a DNA or cDNA) independent of other sequences. The HIV-2 nucleic acid molecules of the disclosure can be RNA, DNA, or include modified forms of either type of nucleic acid. The term includes single and double stranded forms of DNA.

Vectors for cloning and replication of the disclosed HIV-2 nucleic acid molecules include bacterial plasmids. Exemplary bacterial plasmids into which the disclosed nucleic acids can be cloned include E. coli plasmids, such as pBR322, pUC plasmids (such as pUC18 or pUC19), pBluescript, pACYC184, pCD1, pGEM® plasmids (such as pGEM®-3, pGEM®-4, pGEM-T® plasmids; Pomega, Madison, Wis.), TA-cloning vectors, such as pCR® plasmids (for example, pCR® II, pCR® 2.1, or pCR® 4 plasmids; Life Technologies, Grand Island, N.Y.) or pcDNA plasmids (for example pcDNA™3.1 or pcDNA™3.3 plasmids; Life Technologies), and pBAD plasmids. The disclosed nucleic acids can be also be cloned into B. subtilis plasmids, for example, pTA1060 and pHT plasmids (such as pHT01, pHT43, or pHT315 plasmids). The disclosed nucleic acids may also be cloned and/or replicated using viral vectors, such as lambda bacteriophage, M13mp18, or φX174 or yeast vectors, such as pYES, pPIC, and pKLAC1. Many additional plasmids and vectors are available, and can be identified and selected by one of ordinary skill in the art. In some examples, a vector including a disclosed HIV-2 nucleic acid is selected and stored (for example, at less than 0° C., such as −20° C. or −80° C.).

In some examples, a vector including one or more of the HIV-2 nucleic acids disclosed herein (such as SEQ ID NOs: 1-22) is transduced or transformed into a cell. One of ordinary skill in the art can introduce a vector and any inserted sequences into a cell using techniques known in the art. In one non-limiting example, the vector is a bacterial plasmid and includes one or more of the disclosed HIV-2 sequences. The vector is transformed into bacterial cells (such as E. coli) by heat shock or electroporation. Cells including the plasmid can be selected (for example using selection for an antibiotic resistance gene or other selective marker present on the plasmid) and the plasmid including the HIV-2 nucleic acid can be isolated. In some examples, cells transformed with the plasmid of interest are selected and stored (for example, at −80° C.).

Also disclosed herein are methods for amplifying an HIV-2 LTR nucleic acid or an HIV-2 pol nucleic acid (e.g., SEQ ID NOs: 1-22). The methods include contacting a sample including HIV-2 nucleic acids (such as a sample from a subject infected with HIV-2 or an HIV-2 viral isolate) with two or more primers (such as a pair of primers) capable of hybridizing to an HIV-2 nucleic acid under conditions sufficient for amplification of the HIV-2 nucleic acid. In some examples, the method includes amplifying an HIV-2 LTR nucleic acid by contacting a sample including an HIV-2 nucleic acid with at least one forward primer at least 90% identical to (such as at least 91%, 92%, 93%, 94%, 95%, 96%, 97%, 98%, 99%, or 100% identical to) a forward primer selected from SEQ ID NOs: 36-38 and at least one reverse primer at least 90% identical to (such as at least 91%, 92%, 93%, 94%, 95%, 96%, 97%, 98%, 99%, or 100% identical to) a reverse primer selected from SEQ ID NOs: 39-43. In some examples, the method includes amplifying an HIV-2 pol nucleic acid by contacting a sample including an HIV-2 nucleic acid with at least one forward primer at least 90% identical to (such as at least 91%, 92%, 93%, 94%, 95%, 96%, 97%, 98%, 99%, or 100% identical to) a forward primer selected from SEQ ID NOs: 44-46 and at least one reverse primer at least 90% identical to (such as at least 91%, 92%, 93%, 94%, 95%, 96%, 97%, 98%, 99%, or 100% identical to) a reverse primer selected from SEQ ID NOs: 47-52.

The present disclosure is illustrated by the following non-limiting Examples.

### Example 1

### RT-LAMP Assay for Detection of HIV-2

This example describes detection of HIV-2 Group A and B nucleic acids using an RT-LAMP assay.

### Materials and Methods

HIV-1/2 Isolates:

Twelve HIV-2 primary virus isolates, characterized previously (Owen et al., J. Virol. 72:5425-5432, 1998; Masciotra et al., J. Clin. Microbiol. 40:3167-3171, 2002), were used to evaluate the performance of the HIV-2 RT-LAMP assay. The virus stocks were expanded in phytohemagglutinin (PHA)-stimulated peripheral blood mononuclear cells (PBMCs), as described (Owen et al., J. Virol. 72:5425-5432, 1998). Primary HIV-1 isolates of diverse Group M subtypes (Pau et al., J. Virol. Methods 164:55-62, 2010; Gao et al., J. Virol. 72:5680-5698, 1998) were tested to determine assay specificity. RNA extractions were performed on all virus stocks using a QIAamp® Viral RNA Mini Kit (QIAGEN, Valencia, Calif.), according to the manufacturer's instructions. The HIV-1/2 isolates evaluated in this study are listed in Table 3, below.

HIV-2 Linearity Panels:

To evaluate the sensitivity of the HIV-2 RT-LAMP assay for RNA, a linearity panel was created using RNA extracted from HIV-2 NIH-Z purified virus (Advanced Biotechnologies Inc., Columbia, Md.). Virus particle count was provided by the manufacturer and used to quantify RNA copy number. RNA was extracted from the virus stock using a QIAamp® Viral RNA Mini Kit (Qiagen, Valencia, Calif.). The extracted HIV-2 NIH-Z RNA was diluted in RNase-free water to create a panel ranging from 10⁶ to 10² RNA copies/mL.

Given the lack of available commercial HIV-2 DNA quantitative standards, HIV-2 Pol clones were generated from the HIV-2 primary virus isolates. Amplification of the entire Pol gene from the extracted RNA and subsequent cloning of the Pol insert into TOPO® TA cloning vectors (Life Technologies, Grand Island, N.Y.) was performed as described in Example 2. The resulting DNA clones were linearized with a restriction enzyme that recognizes a single restriction site within the vector and no sites within the insert. The restriction digests were incubated overnight at 37° C., using SacI, NotI, or NcoI restriction enzymes (New England Biolabs, Ipswich, Mass.) and the appropriate buffer specified by the manufacturer. The linearized constructs were quantified using a Quant-iT™ PicoGreen® dsDNA Assay Kit (Life Technologies) and DNA copy number/mL was calculated using the following formula: (concentration in ng/mL×6.022×10²³)/(length of template in base pairs×10⁹×650). A DNA linearity panel of 10⁶ to 10² DNA copies/mL was created by diluting each clone in RNase-free water to the specified concentrations.

RT-LAMP Primer Design:

Due to the sequence diversity of the major circulating HIV-2 groups A and B (Kannangai et al., Clin. Infect. Dis. 52:780-787, 2011), two separate sets of HIV-2 integrase-specific primers were designed. Six RT-LAMP primers, forward outer (F3), backward outer (B3), forward inner (FIP), backward inner (BIP), and loop primers (Loop F and Loop B), were generated using the PrimerExplorer V4 software (available on the World Wide Web at primerexplorer.jp/e/). The HIV-2 ROD sequence (GenBank accession number M15390) was used as a reference for generating an initial primer set directed against a conserved region within the integrase gene. Based on the consensus sequences for groups A and B (available on the World Wide Web at hiv.lanl.gov/content/index), nucleotide modifications were made to design two separate primers sets, specific for each group (Table 1). Additional modifications included the insertion of a four thymidine spacer inserted between F2/B2 and F1c/B1c sequences of the FIP and BIP primers, as described (Notomi et al., Nucl. Acids Res. 28:E63, 2000).

For endpoint detection of target-specific amplicons, a fluorescent label (HEX) was added to the 5′ end of the Loop B primer. A quencher probe, composed of a complimentary sequence to the Loop B primer and a Black Hole Quencher™ (BHQ) molecule on the 3′ end, was designed to quench the fluorescence of unbound primer, as described (Curtis et al., J. Med. Virol. 81:966-972, 2009).

TABLE 1
HIV-2 RT-LAMP primer sequences

| | Primer | | SEQ |
|---|---|---|---|
| Group | Name | Primer Sequence (5′-3′) | ID NO: |
| A | F3 | CCTTACAATCCACAAAGCCAA | 23 |
| | B3 | ATTGTATTTCTTGTTCTGTGGTG | 24 |
| | FTP | CTGTATTTGCCTGYTCTCTAATTCTTTTTTAGTAG | 25 |
| | | AAGCAATGAATCACC | |
| | BIP | AGTACTAATGGCAGTTCATTGCATGTTTTGTCTT | 26 |
| | | TCTGCTGGGGTCAT | |
| | Loop F | ACTTATCTGATTTTTTAG | 27 |
| | Loop B | HEX-AATTTTAAAAGAAGGGGAGGA | 28 |
| B | F3 | CCCTATAACCCACAAAGTCAG | 29 |
| | B3 | ATTGTATTTCTTGTTCTGTGGTT | 30 |
| | FTP | TTGATACTGCCTGRTCTCTGATTCTTTTTTAGTAG | 31 |
| | | AAGCAATGAACCATC | |
| | BIP | TGTACTAATGGCAGCTCACTGCATGTTTTGTCTT | 32 |
| | | TCTGCAGGGGTCAT | |
| | Loop F | GTCTATTTGATTTTTTAG | 33 |
| | Loop B | HEX-AATTTTAAAAGAAGGGGAGGA | 34 |
| | Quencher | CCTTCTTTTAAAATT-BHQ1 | 35 |

Real-time RT-LAMP: The RT-LAMP reaction was performed using a total reaction volume of 25 μl containing: 0.2 μM each of F3 and B3 primers, 1.6 μM each of FIP and BIP primers, 0.8 μM each of Loop F and Loop B primers, 0.8 M betaine (Sigma-Aldrich, St. Louis, Mo.), 10 mM MgSO₄, 1.4 mM dNTPs, 1× ThermoPol reaction buffer (New England Biolabs), 12 U Bst DNA polymerase (New England Biolabs), and 2 U AMV reverse transcriptase (Life Technologies). The primer concentration reflects the total amount of each type of primer added, as the primer stocks were made up of a 1:1 ratio of the group A- and B-specific primers. Each reaction contained 15 μl of reaction mix and 10 μl of extracted DNA or RNA. HIV-1 negative controls were included in each run: extracted DNA from 0M10.1 cells (Butera et al., J. Virol. 65:4645-4653, 1991) or RNA from BaL virus stock (Advanced Biotechnologies Inc., Columbia, Md.). For real-time detection, 1 μL of PicoGreen (Life Technologies), diluted 1:100 in TE buffer (200 mM Tris-HCl, 20 mM EDTA), was added to each reaction tube, along with 15 μL of mineral oil. The amplification reaction was carried out in an ESEQuant Tube Scanner (QIAGEN) for 70 minutes at 60° C.

The Tube Scanner was programmed to take a fluorescent reading every 20 seconds. The amplification curves were plotted as fluorescent intensity of PicoGreen (mV) over time (minutes). Sample positivity was determined by the slope validation criteria of the instrument, where the amplification curve exceeded a rate of 20 mV/minute for a minimum of two readings to be deemed positive. The time to positivity was defined as the time point where the amplification curve of a sample met the slope criteria. Target specific amplification was confirmed by endpoint fluorescence of the reaction tube, mediated by the fluorescent-labeled Loop B primer, and by gel electrophoresis on a 3% agarose gel.

To determine the sensitivity of HIV-2 RT-LAMP for DNA and RNA and performance with virus isolates, all linearity panels and virus isolate RNA were tested twice in the Tube Scanner and the average time to positivity of the two separate runs was calculated for each sample. Further testing was performed on the NIH-Z RNA panel members that were at or close to the limit of detection of the assay, as determined by initial testing in the Tube Scanner. Ten replicates of each selected panel member were tested in the Stratagene MX3000P real-time qPCR system, given the limited sample capacity of the Tube Scanner.

Rt-PCR:

For sensitivity comparison with HIV-2 RT-LAMP, the NIH-Z RNA linearity panel and HIV-1/2 virus isolates were tested by RT-PCR using primers that were designed based on a highly conserved region within the HIV-2 integrase gene (Masciotra et al., J. Clin. Microbiol. 40:3167-3171, 2002). The primers also cross-react with HW-1 and SIV sequences. An initial reverse transcription step was performed with the following components: 1× GeneAmp® PCR Buffer II (Applied Biosystems, Grand Island, N.Y.), 5 mM MgCl₂, 1 mM PCR nucleotide mix (Roche Applied Science, Indianapolis, Ind.), 1 μM of primary reverse primer, 50 U RNase Inhibitor (Applied Biosystems), 50 U MuLV Reverse Transcriptase (Applied Biosystems), 9.8 μL of extracted RNA, and RNase-free water (for a final reaction volume of 20 μL). The reaction mixture was heated at 42° C. for 20 minutes, 99° C. for 5 minutes, and 5° C. for 5 minutes. Following cDNA synthesis, nested PCR was performed with the 20 μl product from the reverse transcription step and the following components: 0.25 μM of each forward and reverse primer, 1× GeneAmp® PCR Buffer II, 2.5 mM MgCl₂, 0.2 mM PCR nucleotide mix, 2.5 U of AmpliTaq Gold DNA polymerase (Applied Biosystems), and distilled water (for a final reaction volume of 100 μL). For the second round of PCR, 2 μL of the first (primary) reaction was added to the reaction mix. Both rounds of PCR were performed as follows: 10 minute activation step at 95° C.; 35 cycles of 94° C. for 30 seconds, 50° C. for 30 seconds, and 72° C. for 1 minute; and a final extension cycle at 72° C. for 5 minutes. Amplified products were analyzed by gel electrophoresis on a 1.2% agarose gel. PCR amplification of DNA from the primary HIV-2 isolates has been demonstrated (Masciotra et al., J. Clin. Microbiol. 40:3167-3171, 2002).

HIV-1/2 Multiplex RT-LAMP:

To determine the ability to amplify and differentiate HW-1 and HIV-2 in a single reaction, a multiplex reaction was performed with HIV-1 and HIV-2 specific RT-LAMP primers. The sequence of HIV-1 RT-LAMP primers, directed against a conserved region within the reverse transcriptase (RT) gene, has been described elsewhere (Curtis et al., PLoS One 7:e31432, 2012). To facilitate naked-eye distinction in fluorescence between the two targets, FAM and CalRED590 fluorophores were added to the Loop B primers of the HIV-2 and HIV-1 primers, respectively. The multiplex RT-LAMP reaction was performed as described above for HIV-2, with the addition of the HIV-1 RT primers at a 1:1 ratio with the HIV-2 primers. The amplification reaction was carried out in the presence of one or both targets, which included 10⁵ copies/mL of extracted RNA from HIV-2 NIH-Z and/or HIV-1 BaL virus stocks. Fluorescence of the reaction tubes was visualized with the aid of a UV transilluminator. Additionally, an endpoint fluorescent reading was obtained with the Tube Scanner, using dual fluorescent channel detection. The background fluorescence of the reaction mix, in the absence of target, was subtracted from all tube measurements.

### Results

HIV-2 RT-LAMP Sensitivity and Specificity:

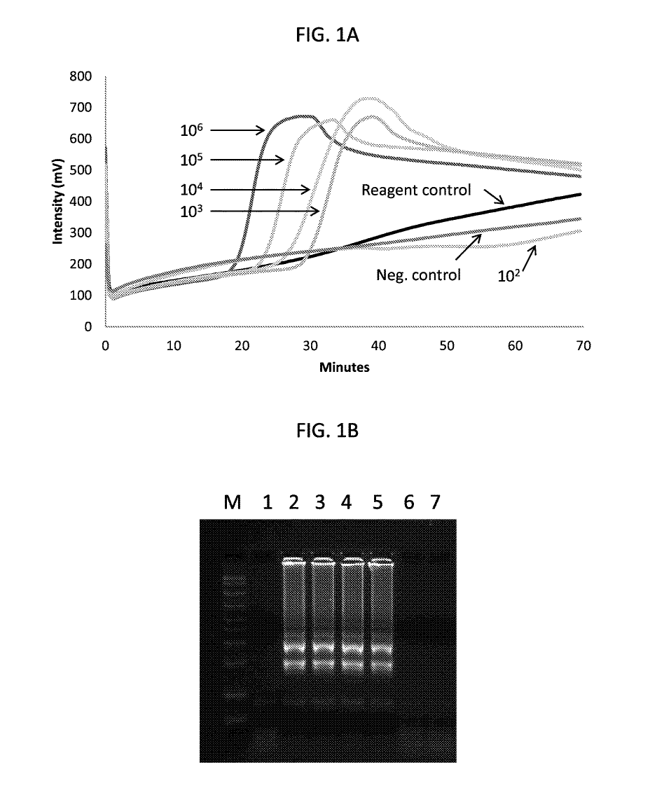

The limit of detection of HIV-2 RT-LAMP for RNA was 10³-10² RNA copies/mL, as measured by the Tube Scanner (FIG. 1A). The characteristic laddering pattern of the LAMP amplicon was confirmed by agarose gel electrophoresis (FIG. 1B). The sensitivity of the assay for RNA was further validated by testing ten additional replicates of the 10⁴-10² copies/mL NIH-Z linearity panel members (Table 2). Overall, 100% of the replicates were detected at 10⁴ copies/mL, while 9/12 (75%) and 2/12 (17%) were detected for the 10³ copies/mL and 10² copies/mL panel members, respectively. The average time to positive RT-LAMP result ranged from 18.3 to 35.5 minutes, for 10⁶ to 10² copies/mL. HIV-2 integrase RT-PCR exhibited similar results to RT-LAMP, detecting all linearity panel members to 10³ RNA copies/mL (Table 2).

TABLE 2
RNA amplification by real-time HIV-2 RT-LAMP

Real-Time RT-LAMP

| Copies/mL | RT-PCR | Result^{a} | Time to Positivity^{b} |
|---|---|---|---|
| 10⁶ | + | 2/2 | 18.3 |
| 10⁵ | + | 2/2 | 20 |
| 10⁴ | + | 12/12 | 22.6 |
| 10³ | + | 9/12 | 30 |
| 10² | − | 2/12 | 35.5 |

^{a}Number positive out of total number tested
^{b}Average time (minutes) of all replicates

All twelve primary HIV-2 isolates of groups A, B, and A/B were positive by both RT-PCR and RT-LAMP (Table 3). For the real-time RT-LAMP, all isolates were amplified in less than 30 minutes, with a median time to positive result of 17.3 minutes. All HIV-1 isolates were negative by RT-LAMP; however, all 12 were RT-PCR positive (Table 3).

TABLE 3
Detection of RNA from HIV clinical isolates

| HIV-2 Isolate | Group | RT-PCR | RT-LAMP | Time to Positivity^{a} |
|---|---|---|---|---|
| A2270 | A | + | + | 15 |
| SLRHC | A | + | + | 22.3 |
| 7924A | A | + | + | 20.8 |
| A2267 | A | + | + | 18.3 |
| 77618 | A | + | + | 18.8 |
| A1958 | A | + | + | 20 |
| GB87 | A | + | + | 13.8 |
| GB122 | A | + | + | 15.5 |
| 60415K | A | + | + | 16.3 |
| 310072 | B | + | + | 15.3 |
| 310319 | B | + | + | 27.3 |
| 7312A | A/B | + | + | 15.8 |
| MEDIAN | | | | 17.3 |

| HIV-1 Isolate | Subtype | RT-PCR | RT-LAMP |
|---|---|---|---|
| 92US657 | B | + | − |
| 92HT593 | B | + | − |
| 92US660 | B | + | − |
| 92US727 | B | + | − |
| 92US714 | B | + | − |
| 93US151 | B | + | − |
| 92RW026 | A | + | − |
| 93MW959 | C | + | − |
| 92UG001 | D | + | − |
| CMU02 | AE | + | − |
| 93BR029 | F | + | − |
| HIV-1 G3 | G | + | − |

^{a}Average time (minutes) of two separate RT-LAMP runs

The limit of detection of HIV-2 real-time RT-LAMP for DNA varied depending on the specific DNA clone. All clones were detected at 10⁴ DNA copies/mL, 7/10 (70%) clones were positive at 10³ copies/mL, and 3/10 (30%) were positive at 10² copies/mL. The median time to positive result for all clones ranged from 22.8 to 43.5 minutes, from 10⁶ to 10³ copies/mL (Table 4).

TABLE 4
HIV-2 Pol DNA clones

| | | Time to Positivity^{a} | | | | | |
|---|---|---|---|---|---|---|---|
| Isolate | Subtype | NIH-Z 10⁶ | 10⁶ | 10⁵ | 10⁴ | 10³ | 10² |
| A2270 | A | 18 | 25.8 | 21.8 | 26.3 | 35 | 34.5^{b} |
| A2267 | A | 19.5 | 31.3 | 36.5 | 54.5 | NEG | NEG |
| 77618 | A | 20 | 22 | 36.8 | 56.3 | NEG | NEG |
| A1958 | A | 19 | 30 | 34.3 | 48.8 | 59.8 | 69.5 |
| GB87 | A | 19.8 | 19.75 | 24 | 31.3 | 44.5 | NEG |
| GB122 | A | 20 | 22 | 26 | 32.8 | NEG | NEG |
| 60415K | A | 23.8 | 25.5 | 31.3 | 45.5 | 60.5 | NEG |
| 310072 | B | 20 | 20.3 | 22.8 | 26 | 42 | 31.5^{b} |
| 310319 | B | 18.8 | 20.8 | 23.3 | 33 | 42 | NEG |
| 7312A | A/B | 18.3 | 22.8 | 27.3 | 49.8 | 43.5 | NEG |
| MEDIAN | | 19.5 | 22.8 | 27.3 | 39.3 | 43.5 | 34.5 |

^{a}Average time (minutes) of two separate runs
^{b}Only one of two replicates was positive

HIV-1/2 Multiplexed RT-LAMP:

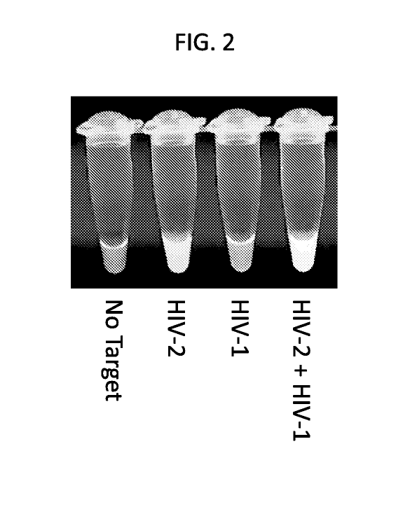

Amplification of both HIV-1 and HIV-2 RNA targets was observed with a multiplexed HIV-1/2 RT-LAMP reaction (FIG. 2). In the presence of a single target (HIV-2 or HIV-1), amplification was confirmed by observing the fluorescence associated with the respective primer set: HIV-2 amplification was indicated by green fluorescence and HIV-1 amplification yielded a red fluorescence. When equal concentrations of HW-1 and HIV-2 RNA were added to the reaction, the resulting fluorescence of the reaction tube was yellow-orange. In the absence of a specific target, no fluorescence was observed. Endpoint fluorescent readings obtained from the Tube Scanner confirmed the presence of amplified targets (Table 5).

TABLE 5
Endpoint fluorescent readings

| Target | FAM (mV) | CALRED (mV) |
|---|---|---|
| No target | 0 | 0 |
| HIV-2 | 26317.6 | 0 |
| HIV-1 | 0 | 394.6 |
| HIV-2 + HIV-1 | 55554.7 | 205.5 |

### Example 2

### Panel of Cloned HIV-2 Nucleic Acids

This example describes isolation of LTR and pol nucleic acids from multiple HIV-2 isolates and a real-time PCR assay for detecting HIV-2 nucleic acids.

### Materials and Methods

HIV-2 Primary Isolates:

Eleven viral stocks of HIV-2 isolates (group A (n=8), group B (n=2), and group AB (n=1)) from various West African countries, including the Ivory Coast, Senegal, and Guinea-Bissau, were used to clone the entire LTR and pol regions from each virus. The demographic characteristics of the patients and establishment of these viral stocks have been previously described (Owen et al., J. Virol., 72:5425-5432, 1998).

Nucleic Acid Extraction from HIV-2 Viral Stocks:

Total RNA was extracted from all viral stocks using the QIAamp® Viral RNA Mini Kit (QIAGEN, Valencia, Calif.). Briefly, 100 μL of virus stocks were obtained from frozen vials of supernatant fluids collected from HIV-2 infected PBMC cultures. Virus stocks were mixed with 560 μL of kit lysis buffer, and were incubated at room temperature for 10 minutes. The mixture was added to 500 μL of 100% ethanol and passed through an RNA-binding column, according to the kit protocol. Total nucleic acid was eluted from the column in 60 μL of kit elution buffer.

Amplification of HIV-2 and Cloning:

RT-PCR amplification of HIV-2 RNA was performed using six sets of primers specific for HIV-2 LTR and pol sequences (Table 6) with the SuperScript™ III One-Step RT-PCR System which includes the Platinum® Taq High Fidelity (Life Technologies, Foster City, Calif.). The RT-PCR conditions for the amplification of the LTR region were as follows: reverse-transcription (RT) at 55° C. for 30 minutes followed by denaturation at 94° C. for 2 minutes. Target amplification consisted of 40 cycles of denaturation at 94° C. for 15 seconds, annealing at 50° C. for 30 seconds, and elongation at 68° C. for 1 minute; with a final extension step at 68° C. for 10 minutes. The RT-PCR conditions for the pol region were similar except for the initial RT step which was 50° C. for 30 minutes, followed by annealing at 55° C. for 30 seconds, and elongation at 68° C. for 3 minutes. The RT-PCR products (LTR; 849-984 bp, pol; 2945-3235 bp) were directly inserted into TOPO® TA plasmids (Life Technologies), according to the manufacturer's instructions, and were transformed into E. coli (TOP10 chemically competent cells from Life Technologies).

TABLE 6
RT-PCR primers for amplification of HIV-2 LTR
and pol

| Primer | | SEQ ID |
|---|---|---|
| Name | Primer Sequence | NO: |
| LTRF1 | TGGAAGGGATGTTTTACAGTGAG | 36 |
| LTRF2 | TGGAAGGGATTTACTATAGTGAGAGA | 37 |
| LTRF3 | TGGAAGGGATTTTTTATAGTGAAAGAAGAC | 38 |
| LTRR1 | GGATTTTCCTGCCTTGGTTT | 39 |
| LTRR2 | TCCCGCTCCTCACGCTG | 40 |
| LTRR3 | CAGGAAAATCCCTAGCAGGTTG | 41 |
| LTRR4 | TGCTAGGGATTTTCCTGCCTCCGTTTC | 42 |
| LTRR5 | CAACCTGCTAGGGATTTTCCTG | 43 |
| PolF1 | CAACAGCACCCCCAGTAGAT | 44 |
| PolF2 | GGAAAGAAGCCTCGCAACTT | 45 |
| PolF3 | AGCCAAGCAATGCAGGGCTCCTAG | 46 |
| PolR1 | ATCTTGGCTTTCCTRCTTGG | 47 |
| PolR2 | GGCACTACAATCCAATTCTT | 48 |
| PolR3 | TGCAAGTCCACCAAGCCCAT | 49 |
| PolR4 | ATAGTCRRTGATGATCTTYGCRTTCCT | 50 |
| PolR5 | CCAAGTGGGAACCACTATCC | 51 |
| PolR6 | GTTGCAATTCTCCTGTTCTATGCTTCAGAT | 52 |

Two clones (3100319pol and 310072pol) required additional sub-cloning modifications due to protein toxicity whereby the resulting recombinant protein caused death of the transformed E. coli cells. For these two clones, colonies that were identified as containing the proper insert were cultured in LB Broth (0.8% sodium chloride) at 32° C. overnight. Nucleic acid from the clones was purified using the QuickLyse Miniprep Kit (QIAGEN), according to the manufacturer's instructions. The purified insert pieces were digested with EcoRI and reinserted into the same vector at a ratio of 4:1 (insert:vector) to prevent expression of the insert. The incubation temperature of the transformed E. coli on solidified LB agar plates and in LB broth was set at 32° C. overnight instead of the standard 37° C.

DNA Sequencing and Phylogenetic Analysis:

The plasmids were purified from each clone using the QuickLyse Miniprep Kit (QIAGEN), according to the package insert. The plasmid DNA extracts were directly sequenced using the BigDye® Terminator v1.1 on an automated ABI Genetic Analyzer 3100 (Life Technologies). Sequences were aligned using CLUSTAL W (Thompson et al., 1994) with representatives of the SIVsmm/HIV-2 lineage from the Los Alamos HIV/SIV Sequence Database (GenBank accession numbers were as follows: for HIV-2A/M30502, U38293, D00835, AF082339; HIV-2B/AB485670, L07625, x61240; HW-2AB/EUO28345). The final alignments after gap-stripping yielded 764 nucleotides for LTR and 2877 nucleotides for pol. Trees were inferred by neighbor-joining (Saitou et al., Mol. Biol. Evol. 4:406-425, 1987) and the evolutionary distances were computed using the Kimura 2-parameter method (Kimura et al., J. Mol. Evol. 16:111-120, 1980) with 1000 bootstrap replicates (Felsenstein, Evolution 39, 783-791, 1985). The evolutionary analyses were conducted using MEGA5 (Tamura et al., Mol. Biol. Evol. 28:2731-2729, 2011).

Nucleotide Sequence Accession Numbers:

The GenBank accession numbers for the sequences determined in this study are 7312ALtr: GenBank/EMBL accession number KF156809; 7924ALtr: KF156810; 60415KLtr: KF156811; 77618Ltr: KF156812; A1958Ltr: KF156813; A2270Ltr: KF156815; GB87Ltr: KF156816; GB122Ltr: KF156817; SLRHCLtr: KF156818; 310072Ltr: KF156819; 310319Ltr: KF156820; 7312APol: KF156821; 7924APol: KF156822; 60415KPol: KF156823; 77618Pol: KF156824; A1958Pol: KF156825; A2270Pol: KF156826; GB87Pol: KF156827; GB122Pol: KF156828; SLRHCPol: KF156829; 310072Pol: KF156830; 310319Pol: KF156831.

Real-Time PCR Amplification of HIV-2 Plasmids:

Using the HIV-2 LTR and pol sequences identified in this study and those from the Los Alamos HIV/SIV Sequence Database (24 LTR and 26 pol), four sets of primers and Taqman probes (two in LTR (LTR1 and LTR2), one in protease (Pro), and one in integrase (Int) region) were designed for real-time amplification and detection of HIV-2 DNA sequences (Table 7).

TABLE 7
Real-time PCR primers and probes

| | Amplicon | | Conc. | | SEQ ID |
|---|---|---|---|---|---|
| Gene | Size (bp) | Type* | (μM) | Sequence | NO: |
| H2LTR1 | 85 | F | 0.3 | CGGAGAGGCTGGCAGATYGAG | 53 |
| | | F | 0.3 | GGCAGAGGCTGGCAGATTGAG | 54 |
| | | P | 0.18 | CalRed610-TCCAGCACTAGCAGG | 55 |
| | | | | TAGAGCC-BHQ2 | |
| | | P | 0.18 | CalRed610-CTCCAGCACTARCAG | 56 |
| | | | | GTAGAGCCT-BHQ2 | |
| | | R | 0.4 | GGTGAGAGTCYAGCAGGGAACA | 57 |
| | | | | C | |
| H2LTR2 | 87 | F | 0.4 | GTGTGTGTTCCCATCTCTCCTAG | 58 |
| | | | | TCG | |
| | | F | 0.4 | GTGTGTGYTCCCATCTCTCCTAG | 59 |
| | | | | TCG | |
| | | P | 0.2 | CalRed610-ACACCGARTGACCAG | 60 |
| | | | | GCGGC-BHQ2 | |
| | | P | 0.2 | CalRed610- CCGCCTGGTCATYCG | 61 |
| | | | | GTGTTCA-BHQ2 | |
| | | P | 0.2 | CalRed610-CCGCCTGGTCATTCG | 62 |
| | | | | GTGCTCC-BHQ2 | |
| | | R | 0.3 | GCAGAAAGGGTCCTAACAGACC | 63 |
| | | | | AGG | |
| | | R | 0.3 | GCRAGAAGGGTCCTAACAGACC | 64 |
| | | | | AGG | |
| H2PRO | 87 | F | 0.3 | CACCACACAGAGAGGCGACAGA | 65 |
| | | | | GGA | |
| | | F | 0.3 | CACCATGCAGGGARACGACAGA | 66 |
| | | | | GGA | 67 |
| | | F | 0.3 | GACCCTACAAGGAGGTGACRGA | |
| | | | | GGA | |
| | | P | 0.16 | CalRed610-TGCTGCACCTCAATT | 68 |
| | | P | 0.16 | CalRed610-TGCTGTGCCTCAATT | 69 |
| | | | | CTCTCTTTGG-BHQ2 | |
| | | R | 0.3 | TGACCCTCRATGTRTGCTGTGAC | 70 |
| | | | | TACTGGTC | |
| | | R | 0.3 | TGACCCTCRATACATGCTTTGAC | 71 |
| | | | | TACTGGTC | |
| | | R | 0.3 | TGACCCTCGATATATGCTTGGAC | 72 |
| | | | | TACTGGTC | |
| H2INT | 111 | F | 0.3 | TAATGGCAGYTCAYTGCATGAA | 73 |
| | | | | TTTTAAAAG | |
| | | F | 0.3 | TRATGGCAACWCACTGCATGAA | 74 |
| | | | | TTTTAAAAG | |
| | | F | 0.3 | AGTAYTAATGGCAGTTCAYTGC | 75 |
| | | | | ATGAATTT | |
| | | F | 0.3 | TGTACTAATGGCAGCTCAYTGC | 76 |
| | | | | ATGAATTT | |
| | | P | 0.18 | CalRed610-TCATATCCCCTATTCC | 77 |
| | | | | TCCCCTTC-BHQ2 | |
| | | P | 0.18 | CalRed610- AGGGGAGGAATAGG | 78 |
| | | | | GGATATGACYCC-BHQ2 | |
| | | R | 0.3 | GGAGGAATTGTATYTCTTGTTCT | 79 |
| | | | | GTGGTRAT | |
| | | R | 0.3 | GGARGAATTGTATTTCTTGTTCT | 80 |
| | | | | GTRGTTAT | |
| | | R | 0.3 | GGAAGAACTGTATTTCTTGCTCT | 81 |
| | | | | GTGGTTAT | |
| | | R | 0.3 | GGAGGAATTGTATTTCTTGTTCT | 82 |
| | | | | GTGGTIATCAT | |
| | | R | 0.3 | GGAAGAATTGTATTTCTTGYTCT | 83 |
| | | | | GTGGTTATCAT | |
| env | 94 | F | | CTCGGACTTTAYTGGCCGGGA | 84 |
| | | F | | CCCGGACTTTAYTGGCTGGGA | 85 |
| | | P | | CalRed610-AGTGCAGCARCAGC | 86 |
| | | | | AACAGCTG-BHQ2 | |
| | | R | | CCCCAGACGGTCAGYCGCAACA | 87 |
| | | R | | CCCCAGACGGTCAATCTCAACA | 88 |
| LTR-gag | 97 | F | | TTGGCGCCYGAACAGGGAC | 89 |
| | | P | | CalRed610-AGTGARGGCAGTAA | 90 |
| | | | | GGGCGGC-BHQ2 | |
| | | R | | GCACTCCGTCGTGGTTTGTTCCT | 91 |
| | | R | | GCWCTCCGTCGTGGTTGATTCCT | 92 |
| RNase P | 77 | F | | GTGTTTGCAGATTTGGACCTGCG | 93 |
| | | P | | FAM-TGACCTGAAGGCTCTGCG | 94 |
| | | | | CG BHQ2 | |
| | | R | | AGGTGAGCGGCTGTCTCCAC | 95 |

*F, Forward Primer; P, Probe; R Reverse Primer

The plasmids were purified from each clone using the QuickLyse Miniprep Kit (QIAGEN), as directed by the package insert. The total DNA concentration of the plasmid constructs were determined fluorometrically using the Quant-It™ dsDNA high sensitivity assay kit on a Qubit® fluorometer (Life Technologies, Grand Island, N.Y.). The plasmid copy number per mL was estimated using the equation

(A×6.022×10²³)/(B×10⁹×650),

where A is the total DNA concentration in ng/mL and B is the length of the plasmid in number of base pairs (4780 for LTR and 6924 for poly. Serial 1:10 dilutions of each of the plasmids were prepared in Tris buffer containing 0.1 mM EDTA to effect concentrations ranging from 2×10⁵ to 2 copies/μL. These dilutions were used to evaluate the assay performance. Real-time PCR was carried out using the QuantiFast® Multiplex PCR kit (Qiagen, Valencia Calif.) on a Model MX3000P qPCR system (Stratagene, Santa Clara, Calif.). The reaction mixture (25 μL) contained 5 μL of plasmid DNA, 12.5 μl of QuantiFast® reagent mix, 1 μL of the primers and probes mixture (Table 7), and 6.5 μL of deionized water. The amplification reaction was performed using a 1.5 minute enzyme activation at 95° C., followed by 45 cycles of amplification (94° C. for 1 second and 60° C. for 25 seconds).

### Results

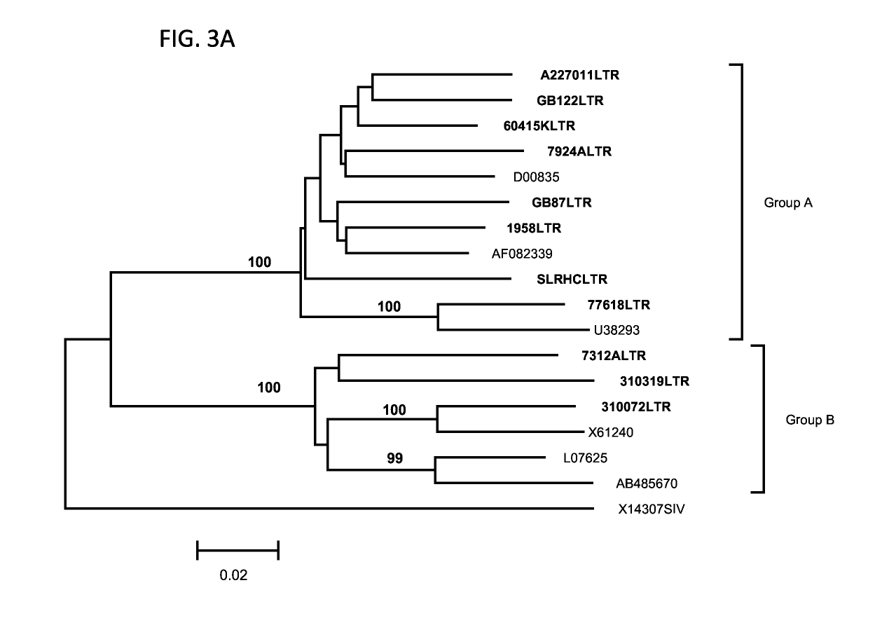

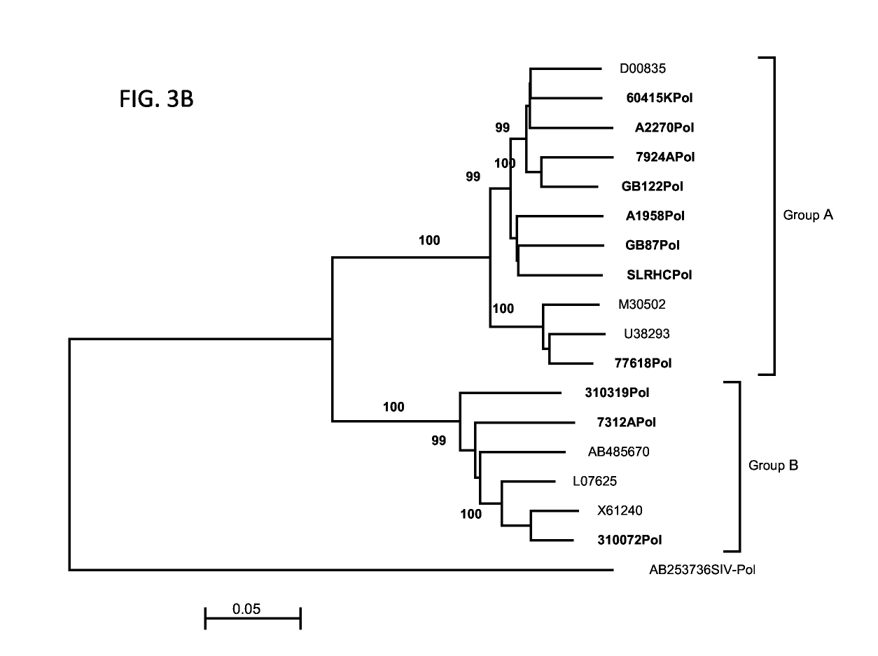

The entire LTR(˜849 bp) and pol (2995 bp) regions for 11 HIV-2 isolates comprising groups A, B, and AB were successfully cloned, sequenced, and group classified (Table 8). In 10 of the 11 isolates, group classification based on the LTR and pol regions was fully concordant with V3-based grouping as described previously (Owen et al., J. Virol., 72:5425-5432, 1998). In both the LTR and pol regions, A2270, GB122, 60415k, 7924A, GB87, 1958, SLRHC, and 77618 were classified as group A, and 7312A, 310319, and 310072 were classified as group B (FIGS. 3A and 3B). The sequence of isolate 7312A was previously reported by Gao et al. as group AB in the env region (Gao et al., Nature 358:495-499, 1992), but both the LTR and the pol sequences were classified as group B in this study.

TABLE 8
Amplification and cloning of LTR and pol HIV-2 nucleic acids

| | | | | | Primers (5′-3′) | |
|---|---|---|---|---|---|---|
| Gene | Isolate | Group | Insert (bp) | Vector | Forward | Reverse |
| LTR | A2270 | A | 849 | 2 | LTRF1 | LTRR1 |
| | SLRHC | A | 849 | 1 | LTRF1 | LTRR1 |
| | 7924A | A | 872 | 1 | LTRF3 | LTRR1 |
| | 77618 | A | 849 | 1 | LTRF1 | LTRR1 |
| | A1958 | A | 862 | 1 | LTRF3 | LTRR4 |
| | GB87 | A | 849 | 2 | LTRF1 | LTRR1 |
| | GB122 | A | 849 | 1 | LTRF1 | LTRR1 |
| | 60415K | A | 849 | 1 | LTRF1 | LTRR1 |
| | 310072 | B | 984 | 1 | LTRF2 | LTRR2 |
| | 310319 | B | 897 | 1 | LTRF3 | LTRR3 |
| | 7312A | A/B* | 872 | 2 | LTRF3 | LTRR5 |
| Pol | A2270 | A | 2945 | 1 | PolF1 | PolR1 |
| | SLRHC | A | 2957 | 1 | PolF1 | PolR4 |
| | 7924A | A | 2995 | 1 | PolF1 | PolR5 |
| | 77618 | A | 2945 | 1 | PolF1 | PolR5 |
| | A1958 | A | 2957 | 1 | PolF1 | PolR4 |
| | GB87 | A | 2945 | 1 | PolF1 | PolR1 |
| | GB122 | A | 2995 | 1 | PolF1 | PolR5 |
| | 60415K | A | 2995 | 1 | PolF1 | PolR5 |
| | 310072 | B | 3043 | 1 | PolF2 | PolR2 |
| | 310319 | B | 3174 | 1 | PolF2 | PolR3 |
| | 7312A | A/B* | 3235 | 1 | PolF3 | PolR6 |

*The entire 7312A gene was sequenced (recombinant of A in env gene with the backbone of a group B genome) (Gao et al., 1992)
Vector 1 = pCR 4-TOPO ® TA;
Vector 2 = pCR2.1-TOPO ® TA

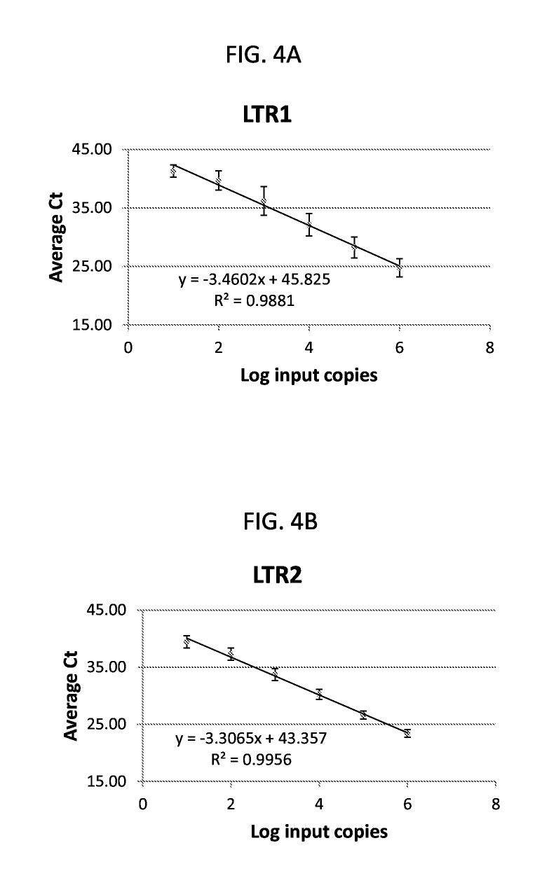

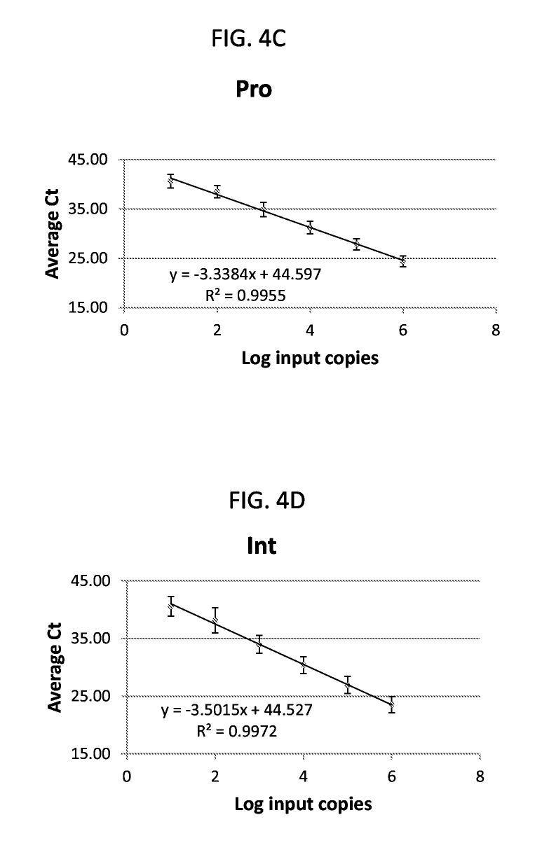

The 22 plasmid dilution panels were used as the templates to evaluate the real-time PCR assay sensitivity of the primer/probes sets developed in this study. Response curves (Cₜ vs. copy number) of the four assays for each of the plasmid panels are shown in FIG. 4A-4D. All four assays detected at least 100 copies, and in most cases 10 copies of the plasmid per reaction (LTR1, 9/11; LTR2, 6/11; Pro, 7/11, and Int, 8/11). The average amplification efficiency for each of the four assays was similar, between 98%-102% as determined by the slopes of the response curves.

### Example 3

### Detection of HIV-2 Nucleic Acids Using RT-LAMP

This example describes particular methods useful for detecting HIV-2 nucleic acids in a sample using an RT-LAMP assay. However, one skilled in the art will appreciate that methods that deviate from these specific methods can also be used to successfully detect HIV-2 nucleic acids in a sample.

Clinical samples are obtained from a subject (such as a subject suspected of having an HIV infection), such as blood, plasma, serum, or oral fluid (saliva, sputum, or oral swab). Typically, the sample is used directly or with minimal processing (for example, dilution and/or vortexing in water, buffer, or lysis buffer). However, RNA can be extracted from the sample using routine methods (for example using a commercial kit) if desired.

RT-LAMP is performed in a reaction including a reaction mix (e.g., buffers, MgCl₂, MnCl₂, dNTPs, reverse transcriptase, and DNA polymerase), sample (e.g., about 1-10 μL of unextracted sample or about 5-10 μL of nucleic acid extracted from the sample), and primers. The primers are included in the reaction as follows: F3 (SEQ ID NOs: 23 and 29) and B3 (SEQ ID NOs: 24 and 30) at 0.1 μM each, FIP (SEQ ID NOs: 25 and 31) and BIP (SEQ ID NOs: 26 and 32) at 08 μM each, and Loop F (SEQ ID NOs: 27 and 33) and Loop B (SEQ ID NOs: 38 and 34) at 0.4 μM each. The assay is incubated at 60° C. for about 60-70 minutes. Samples are examined visually or fluorescence is detected using an instrument such as a real-time PCR platform (e.g., ABI 7500 platform or ESEQuant tube scanner). Positive samples are those with observable fluorescence greater than that in a reagent only (no sample) control tube or other negative control.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of detecting presence of human immunodeficiency virus-2 (HIV-2) nucleic acid in a sample, comprising:
contacting the sample with at least one set of loop-mediated isothermal amplification (LAMP) primers specific for an HIV-2 integrase nucleic acid under conditions sufficient for amplification of the HIV-2 nucleic acid, thereby producing an HIV-2 amplification product; and
detecting the HIV-2 amplification product, thereby detecting presence of HIV-2 nucleic acid in the sample.

2. The method of claim 1, wherein the at least one set of LAMP primers comprises:
a) a set of primers comprising SEQ ID NOs: 23-28; or
b) a set of primers comprising SEQ ID NOs: 29-34.

3. (canceled)

4. The method of claim 1, wherein the at least one set of LAMP primers is specific for a Group A HIV-2 integrase nucleic acid or is specific for a Group B HIV-2 integrase nucleic acid.

5. The method of claim 4, wherein the at least one set of LAMP primers is specific for:
the Group A HIV-2 integrase nucleic acid and comprises primers comprising a nucleic acid sequence at least 90% identical to each of SEQ ID NOs: 23-28; or
the Group B HIV-2 integrase nucleic acid and comprises primers comprising a nucleic acid sequence at least 90% identical to each of SEQ ID NOs: 29-34.

6. The method of claim 5, wherein the set of LAMP primers comprises primers comprising or consisting of the nucleic acid sequence of each of SEQ ID NOs: 23-28.

7. (canceled)

8. The method of claim 5, wherein the set of LAMP primers comprises primers comprising or consisting of the nucleic acid sequence of each of SEQ ID NOs: 29-34.

9. The method of claim 1, wherein at least one primer in the set of LAMP primers comprises a detectable label.

10. The method of claim 9, wherein the detectable label comprises a fluorophore.

11. The method of claim 1, wherein the at least one set of LAMP primers further comprises a quencher oligonucleotide.

12. The method of claim 11, wherein the quencher oligonucleotide comprises or consists of the nucleic acid sequence of SEQ ID NO: 35 and a fluorescence quencher.

13. (canceled)

14. The method of claim 12, wherein the fluorescence quencher comprises a dark quencher.

15. The method of claim 1, further comprising contacting the sample with a reverse transcriptase under conditions sufficient for reverse transcription of the HIV-2 nucleic acid.

16. The method of claim 1, wherein detecting the HIV-2 amplification product comprises turbidity measurement, fluorescence detection, or gel electrophoresis.

17. The method of claim 1, wherein the sample comprises isolated DNA, isolated RNA, blood, urine, saliva, tissue biopsy, fine needle aspirate, or a surgical specimen.

18-28. (canceled)

29. A method of detecting presence of human immunodeficiency virus-2 (HIV-2) in a sample, comprising:
contacting the sample with:
(a) at least one detectably labeled probe capable of hybridizing specifically to an HIV-2 nucleic acid, wherein the probe comprises a nucleic acid sequence at least 90% identical to one of SEQ ID NOs: 55, 56, 60-62, 68, 69, 77, 78, 86, and 90; and
(b) at least one forward primer comprising a nucleic acid sequence at least 90% identical to one of SEQ ID NOs: 53, 54, 58, 59, 65-67, 73-76, 84, 85, and 89, and at least one reverse primer comprising a nucleic acid sequence at least 90% identical to one of SEQ ID NOs: 57, 63, 64, 70-72, 79-83, 87, 88, 91, and 92 wherein the at least one forward primer and at least one reverse primer are capable of amplifying the HIV-2 nucleic acid; and
detecting hybridization of the detectably labeled probe to the HIV-2 nucleic acid, thereby detecting presence of HIV-2 in the sample.

30. The method of claim 29, wherein the HIV-2 nucleic acid comprises:
an LTR nucleic acid and wherein the probe comprises or consists of the nucleic acid sequence of SEQ ID NO: 55 or 56, the forward primer comprises or consists of the nucleic acid sequence of SEQ ID NO: 53 or 54, and the reverse primer comprises or consists of the nucleic acid sequence of SEQ ID NO: 57;
an LTR nucleic acid and wherein the probe comprises or consists of the nucleic acid sequence of any one of SEQ ID NOs: 60-62, the forward primer comprises or consists of the nucleic acid sequence of SEQ ID NO: 58 or 59, and the reverse primer comprises or consists of the nucleic acid sequence of SEQ ID NO: 63 or 64;
a protease-encoding nucleic acid and wherein the probe comprises or consists of the nucleic acid sequence of SEQ ID NO: 68 or 69, the forward primer comprises or consists of the nucleic acid sequence of any one of SEQ ID NOs: 65-67, and the reverse primer comprises or consists of the nucleic acid sequence of any one of SEQ ID NOs: 70-72;
an integrase-encoding nucleic acid and wherein the probe comprises or consists of the nucleic acid sequence of SEQ ID NO: 77 or 78, the forward primer comprises or consists of the nucleic acid sequence of any one of SEQ ID NO: 73-76, and the reverse primer comprises or consists of the nucleic acid sequence of any one of SEQ ID NOs: 79-83;
an env nucleic acid and wherein the probe comprises or consists of the nucleic acid sequence of SEQ ID NO: 86, the forward primer comprises or consists of the nucleic acid sequence of SEQ ID NO: 84 or 85, and the reverse primer comprises or consists of the nucleic acid sequence of SEQ ID NOs: 87 or 88; or
an LTR-gag nucleic acid and wherein the probe comprises or consists of the nucleic acid sequence of SEQ ID NO: 90, the forward primer comprises or consists of the nucleic acid sequence of SEQ ID NO: 89, and the reverse primer comprises or consists of the nucleic acid sequence of SEQ ID NOs: 91 or 92.

31-35. (canceled)

36. The method of claim 29, further comprising contacting the sample with at least one detectably labeled probe capable of hybridizing to a control nucleic acid and a forward primer and a reverse primer capable of amplifying at least a portion of the control nucleic acid and detecting hybridization of the control probe to the control nucleic acid.

37. The method of claim 36, wherein the control nucleic acid comprises a human RNase P nucleic acid and wherein the probe comprises or consists of the nucleic acid sequence of SEQ ID NO: 94, the forward primer comprises or consists of the nucleic acid sequence of SEQ ID NO: 93, and the reverse primer comprises or consists of the nucleic acid sequence of SEQ ID NOs: 95.

38. The method of claim 29, wherein the detectable label comprises a donor fluorophore, an acceptor fluorophore, or a combination thereof.

39. The method of claim 29, wherein the sample comprises isolated DNA, isolated RNA, blood, urine, saliva, tissue biopsy, fine needle aspirate, or a surgical specimen.

40. An isolated nucleic acid probe 20 to 40 nucleotides in length comprising a nucleic acid sequence at least 90% identical to any one of SEQ ID NOs: 55, 56, 60-62, 68, 69, 77, 78, 86, and 90 and a detectable label.

41. The isolated nucleic acid probe of claim 40, wherein the probe comprises or consists of the nucleic acid sequence of any one of SEQ ID NOs: 55, 56, 60-62, 68, 69, 77, 78, 86, and 90 and a detectable label.

42-44. (canceled)

45. A kit for detection of an HIV-2 nucleic acid in a sample, comprising the isolated nucleic acid probe of claim 40.

46. The kit of claim 45, further comprising one or more primers for amplification of an HIV-2 nucleic acid, wherein the one or more primers comprise or consist of the nucleic acid sequence of any one of SEQ ID NOs: 53, 54, 57-59, 63-67, 70-76, 79-85, 87-89, 91, and 92.

47-51. (canceled)

52. A vector comprising an isolated HIV-2 nucleic acid with at least 90% sequence identity to the nucleic acid sequence of any one of SEQ ID NOs: 1-22.

53. The vector of claim 52, wherein the vector comprises a bacterial vector, a yeast vector, a viral vector, or a mammalian vector.

54. A host cell transformed with the vector of claim 52.

55. A method of amplifying an HIV-2 nucleic acid, wherein:
the HIV-2 nucleic acid is an HIV-2 LTR nucleic acid and comprising contacting a sample comprising an HIV-2 LTR nucleic acid with at least one forward primer at least 90% identical to one of SEQ ID NOs: 36-38 and at least one reverse primer at least 90% identical to one of SEQ ID NOs: 39-43 under conditions sufficient to amplify the HIV-2 LTR nucleic acid; or
the HIV-2 nucleic acid is an HIV-2 pol nucleic acid and comprising contacting a sample comprising an HIV-2 pol nucleic acid with at least one forward primer at least 90% identical to one of SEQ ID NOs: 44-46 and at least one reverse primer at least 90% identical to one of SEQ ID NOs: 47-52 under conditions sufficient to amplify the HIV-2 pol nucleic acid.

56. (canceled)
